(19) United States
(12) Patent Application Publication
(10) Pub. No.: US 2009/0118270 A1
(43) Pub. Date: May 7, 2009

(54) PHOSPHODIESTERASE 4 INHIBITORS

(76) Inventors: Francisco Xavier Talamas, Mountain View, CA (US); Joan Marie Caroon, Mountain View, CA (US); Robert Dunn, Towaco, NJ (US); Allen Hopper, Glen Rock, NJ (US); Erik Kuester, Rochester, MN (US); Richard Schumacher, Monroe, NY (US); Ashok Tehim, Ridgewood, NJ (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/329,214

(22) Filed: Dec. 5, 2008

Related U.S. Application Data
(63) Continuation of application No. 11/449,868, filed on Jun. 9, 2006.
(60) Provisional application No. 60/689,060, filed on Jun. 10, 2005.

(51) Int. Cl. A61K 31/5377; C07D 417/12; A61K 31/4439; A61K 31/506; A61P 25/18; A61P 25/24; C07D 401/12; C07D 413/02

(52) U.S. Cl. 514/231.8; 546/269.7; 514/342; 514/256; 544/333; 544/86

(57) ABSTRACT

PDE4 inhibition is achieved by novel compounds, e.g., N-substituted diarylamine analogs. The compounds of the present invention are of Formulas I-III:

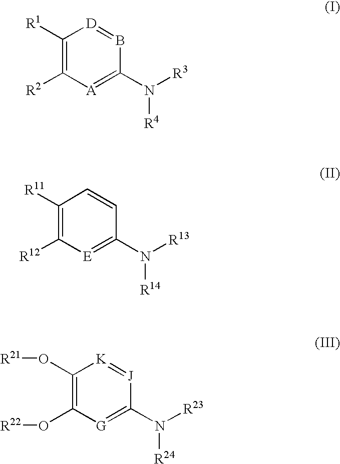

wherein A, B, D, E, G, J, K, R¹, R², R³, R⁴, R¹¹, R¹², R¹³, R¹⁴, R²¹, R²², R²³ and R²⁴ are as defined herein.

This application claims the benefit of U.S. Provisional Application Ser. No. 60/689,060, filed Jun. 10, 2005, the entire disclosure of which is hereby incorporated by reference.

This application is related to U.S. application Ser. No. 11/008,775, filed Dec. 10, 2004, which claims the benefit of U.S. Provisional Application Ser. No. 60/528,486, filed Dec. 11, 2003, the entire disclosure of each of which is hereby incorporated by reference.

### FIELD OF THE INVENTION

The present invention relates generally to the field of phosphodiesterase 4 (PDE4) enzyme inhibition. More specifically, this invention relates to selective PDE4 inhibition by novel compounds, e.g., N-substituted diarylamine analogs, methods of preparing such compounds, compositions containing such compounds, and methods of use thereof.

### BACKGROUND OF THE INVENTION

The cyclic nucleotide specific phosphodiesterases (PDEs) represent a family of enzymes that catalyze the hydrolysis of various cyclic nucleoside monophosphates (including CAMP and cGMP). These cyclic nucleotides act as second messengers within cells, and as messengers, carry impulses from cell surface receptors having bound various hormones and neurotransmitters. PDEs act to regulate the level of cyclic nucleotides within cells and maintain cyclic nucleotide homeostasis by degrading such cyclic mononucleotides resulting in termination of their messenger role.

PDE enzymes can be grouped into eleven families according to their specificity toward hydrolysis of CAMP or cGMP, their sensitivity to regulation by calcium, calmodulin or cGMP, and their selective inhibition by various compounds. For example, PDE1 is stimulated by Ca²⁺/calmodulin. PDE2 is cGMP-dependent, and is found in the heart and adrenals. PDE3 is cGMP-dependent, and inhibition of this enzyme creates positive inotropic activity. PDE4 is cAMP specific, and its inhibition causes airway relaxation, anti-inflammatory, enhanced cognition, and antidepressant activity. PDE5 appears to be important in regulating cGMP content in vascular smooth muscle, and therefore PDE5 inhibitors may have cardiovascular activity. Since the PDEs possess distinct biochemical properties, it is likely that they are subject to a variety of different forms of regulation.

PDE4 is distinguished by various kinetic properties including low Michaelis constant for cAMP and sensitivity to certain drugs. The PDE4 enzyme family consists of four genes, which produce 4 isoforms of the PDE4 enzyme designated PDE4A, PDE4B, PDE4C, and PDE4D [See: Wang et al., Expression, Purification, and Characterization of human cAMP-Specific Phosphodiesterase (PDE4) Subtypes A, B, C, and D, Biochem. Biophys. Res. Comm., 234, 320-324 (1997)]. In addition, various splice variants of each PDE4 isoform have been identified.

PDE4 isoenzymes are localized in the cytosol of cells and specifically inactivate cAMP by catalyzing its hydrolysis to adenosine 5′-monophosphate (AMP). Regulation of cAMP activity is important in many biological processes, including inflammation and memory. Inhibitors of PDE4 isoenzymes such as rolipram, piclamilast, CDP-840 and ariflo are powerful antiinflammatory agents and therefore may be useful in treating diseases where inflammation is problematic such as asthma or arthritis. Further, rolipram improves the cognitive performance of rats and mice in learning paradigms.

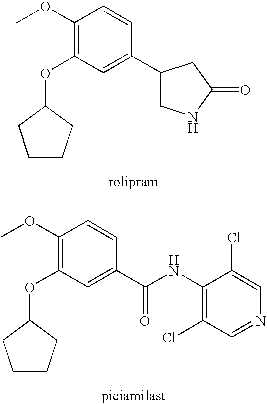

In addition to such compounds as rolipram, xanthine derivatives such as pentoxifylline, denbufyllilne, and theophylline inhibit PDE4 and have received considerable attention of late for their cognition enhancing effects. cAMP and cGMP are second messengers that mediate cellular responses to many different hormones and neurotransmitters. Thus, therapeutically significant effects may result from PDE inhibition and the resulting increase in intracellular cAMP or cGMP in key cells, such as those located in the nervous system and elsewhere in the body.

Rolipram, previously in development as an anti-depressant, selectively inhibits the PDE4 enzyme and has become a standard agent in the classification of PDE enzyme subtypes. Early work in the PDE4 field focused on depression and inflammation, and has subsequently been extended to include indications such as dementia. [see “The PDE IV Family Of Calcium-Phosphodiesterases Enzymes,” John A. Lowe, III, et al., Drugs of the Future 1992, 17(9):799-807 for a general review]. Further clinical developments of rolipram and other first-generation PDE4 inhibitors were terminated due to the side effect profile of these compounds. The primary side effect in primates is emesis, while the primary side effects in rodents are testicular degranulation, weakening of vascular smooth muscle, psychotrophic effects, increased gastric acid secretion and stomach erosion.

### SUMMARY OF THE INVENTION

The present invention relates to novel compounds, e.g., novel N-substituted diarylamine compounds, that inhibit PDE4 enzymes, and especially have improved side effect profiles, e.g., are relatively non-emetic, (e.g., as compared to the previously discussed prior art compounds). Preferably, the compounds selectively inhibit PDE4 enzymes. The compounds of this invention at the same time facilitate entry into cells, especially cells of the nervous system.

Still further, the present invention provides methods for synthesizing compounds with such activity and selectivity as well as methods of (and corresponding pharmaceutical compositions for) treating a patient, e.g., mammals, including humans, requiring PDE inhibition, especially PDE4 inhibition, for a disease state that involves elevated intracellular PDE4 levels or decreased cAMP levels, e.g., involving neurological syndromes, especially those states associated with memory impairment, most especially long term memory impairment, as where such memory impairment is due in part to catabolism of intracellular cAMP levels by PDE4 enzymes, or where such memory impairment may be improved by effectively inhibiting PDE4 enzyme activity.

In a preferred aspect, the compounds of the invention improve such diseases by inhibiting PDE4 enzymes at doses which do not induce emesis.

The present invention includes compounds of Formula I:

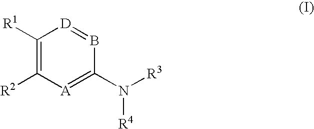

wherein
A, B and D are each, independently, N or CR⁵ wherein at least one of A, B and D is N;
R¹ is halogen, alkyl having 1 to 4 carbon atoms (e.g., methyl, ethyl), halogenated alkyl having 1 to 4 carbon atoms (e.g., CH₂F, CHF₂, CF₃), OR⁶, COR⁶, CONR⁶R¹⁰, or NR⁶COR¹⁰;
R² is halogen, alkyl having 1 to 4 carbon atoms (e.g., methyl, ethyl), halogenated alkyl having 1 to 4 carbon atoms (e.g., CH₂F, CHF₂, CF₃), OR⁷, COR⁶, CONR⁶R¹⁰, or NR⁶COR¹⁰;
R³ is a partially unsaturated carbocycle-alkyl group wherein the carbocyclic portion has 5 to 14 carbon atoms and the alkyl portion which is branched or unbranched has 1 to 5 carbon atoms, wherein the partially unsaturated carbocycle-group is unsubstituted, substituted in the carbocyclic portion one or more times by halogen, alkyl, alkoxy, nitro, cyano, oxo, or combinations thereof, and/or substituted in the alkyl portion one or more times by halogen, C₁₋₄-alkoxy, cyano or combinations thereof (e.g., cyclohexenylmethyl, etc.),
   arylalkyl having 7 to 19 carbon atoms, wherein the aryl portion has 6 to 14 carbon atoms and the alkyl portion, which is branched or unbranched, has 1 to 5 carbon atoms, wherein the arylalkyl radical is unsubstituted or substituted, in the aryl portion, one or more times by halogen, trifluoromethyl, CF₃O, nitro, amino, alkyl, alkoxy, amino, alkylamino, dialkylamino, or combinations thereof, and/or substituted in the alkyl portion by halogen, cyano, alkyl having 1 to 4 carbon atoms (e.g., methyl), or combinations thereof, wherein in the alkyl portion one or more —CH₂CH₂— groups are each optionally replaced by —CH═CH— or —C≡C—, and/or one or more —CH₂— groups are each optionally replaced by —O— or NH— (e.g., benzyl, phenethyl, phenpropyl, methylbenzyl, methoxybenzyl, trifluoromethylbenzyl, methylenedioxobenzyl, etc.), or
   a heterocycle-alkyl group, wherein the heterocyclic portion is saturated, partially saturated or unsaturated (e.g., heteroaryl), and has 5 to 10 ring atoms in which at least 1 ring atom is an N,N—O (that is N-oxide), O or S, the alkyl portion, which is branched or unbranched, has 1 to 5 carbon atoms, the heterocycle-alkyl group is unsubstituted, substituted one or more times in the heterocyclic portion by halogen, alkyl, alkoxy, cyano, trifluoromethyl, CF₃O, nitro, oxo, amino, alkylamino, dialkylamino, or combinations thereof and/or substituted in the alkyl portion one or more times by halogen, cyano, alkyl having 1 to 4 carbon atoms (e.g., methyl), or combinations thereof (e.g., pyridylmethyl, pyridylpropyl, methylpyridylmethyl, chloropyridylmethyl, dichloropyridylmethyl, thienylmethyl, thiazolylmethyl, quinolinylmethyl, isoquinolinylmethyl, piperidinylmethyl, furanylmethyl, imidazolylmethyl, methylimidazolylmethyl, pyrrolylmethyl, etc);
R⁴ is cycloalkyl having 3 to 10, preferably 3 to 8 carbon atoms, which is unsubstituted or substituted one or more times by halogen, hydroxy, oxo, cyano, alkyl having 1 to 4 carbon atoms, alkoxy having 1 to 4 carbon atoms, or combinations thereof (e.g., cyclopentyl),
   aryl having 6 to 14 carbon atoms and which is unsubstituted or substituted one or more times by halogen, alkyl, alkenyl, alkynyl, hydroxy, alkoxy, alkoxyalkoxy, nitro, methylenedioxy, ethylenedioxy, trifluoromethyl, OCF₃, amino, aminoalkyl, aminoalkoxy, dialkylamino, hydroxyalkyl (e.g., hydroxymethyl), hydroxamic acid (—C(O)—NHOH), pyrrolyl, tetrazole-5-yl, 2(-heterocycle)tetrazole-5-yl (e.g., 2-(2-tetrahydropyranyl)tetrazole-5-yl), hydroxyalkoxy, carboxy, carboxyalkyl, alkoxycarbonyl (e.g., tert-butyloxycarbonyl, ethoxycarbonyl), cyano, acyl, alkylthio, alkylsulfinyl, alkylsulfionyl, phenoxy, trialkylsilyloxy (e.g. tert-butyldimethylsilyloxy), R⁸-L-, or combinations thereof (e.g., substituted or unsubstituted phenyl, naphthyl, and biphenyl, such as phenyl, methylphenyl, chlorophenyl, fluorophenyl, vinylphenyl, cyanophenyl, methylenedioxophenyl, ethylphenyl, dichlorophenyl, carboxyphenyl, ethoxycarbonylphenyl, dimethylphenyl, hydroxymethylphenyl, nitrophenyl, aminophenyl, etc.),
   heteroaryl having 5 to 10 ring atoms in which at least 1 ring atom is a heteroatom (e.g., N, S or O), which is unsubstituted or substituted one or more times by halogen, alkyl, hydroxy, alkoxy, alkoxyalkoxy, nitro, methylenedioxy, ethylenedioxy, trifluoromethyl, amino, aminomethyl, aminoalkyl, aminoalkoxy, dialkylamino, hydroxyalkyl (e.g., hydroxymethyl), hydroxamic acid (—C(O)—NHOH), tetrazole-5-yl, hydroxyalkoxy, carboxy, carboxyalkyl, alkoxycarbonyl (e.g., tert-butyloxycarbonyl, ethoxycarbonyl), cyano, acyl, alkylthio, alkylsulfinyl, alkylsulfonyl, phenoxy, trialkylsilyloxy (e.g. tert-butyldimethylsilyloxy), R⁸-L-, or combinations thereof (e.g., pyridyl, thienyl, pyrazinyl, quinolinyl, isoquinolinyl, pyrimidinyl, imidazolyl, thiazolyl, etc.),
   a heterocyclic group, which is saturated or partially saturated, having 5 to 10 ring atoms in which at least 1 ring atom is an N, O or S atom, which is unsubstituted or substituted one or more times by halogen, alkyl, hydroxy, alkoxy, alkoxyalkoxy, nitro, oxo, methylenedioxy, ethylenedioxy, trifluoromethyl, OCF₃, amino, aminomethyl, aminoalkyl, aminoalkoxy, dialkylamino, hydroxyalkyl (e.g., hydroxymethyl), hydroxamic acid (—C(O)—NHOH), tetrazole-5-yl, hydroxyalkoxy, carboxy, alkoxycarbonyl (e.g., tert-butyloxycarbonyl, ethoxycarbonyl), cyano, acyl (e.g., optionally substituted acetyl or optionally substituted benzoyl), alkylthio, alkylsulfnyl, alkylsulfonyl, phenylsulfonyl, phenoxy, cycloalkyl, aryl, heteroaryl or combinations thereof (e.g., piperidinyl, pyrrolydinyl, amidazolidinyl, pyrrolinyl, etc.),
   a heterocycle-alkyl group, wherein the heterocyclic portion is saturated, partially saturated or unsaturated (e.g., heteroaryl), and has 5 to 10 ring atoms in which at least 1 ring atom is an N, O or S atom, and the alkyl portion is branched or unbranched and has 1 to 5 carbon atoms, the heterocycle-alkyl group is unsubstituted, substituted one or more times in the heterocyclic portion by halogen, alkyl, hydroxy, alkoxy, alkoxyalkoxy, nitro, oxo, methylenedioxy, ethylenedioxy, trifluoromethyl, OCF₃, amino, aminomethyl, aminoalkyl, aminoalkoxy, dialkylamino, hydroxyalkyl (e.g., hydroxymethyl), hydroxamic acid (—C(O)—NHOH), tetrazole-5-yl, hydroxyalkoxy, carboxy, alkoxycarbonyl (e.g., tert-butyloxycarbonyl, ethoxycarbonyl), cyano, acyl alkylthio, alkylsulfinyl, alkylsulfonyl, phenylsulfonyl, phenoxy, cycloalkyl, aryl, heteroaryl or combinations thereof, and/or substituted in the alkyl portion one or more times by halogen, oxo, hydroxy, cyano, or combinations thereof, and wherein in the alkyl portion one or more —CH₂CH₂— groups are each optionally replaced by —CH═CH— or —C≡C—, and one or more —CH₂— groups are each optionally replaced by —O— or —NH— (e.g., pyridylethyl, pydridylpropyl, methylpiperazinylethyl, piperidinylmethyl, pyrrolydinylmethyl, amidazolidinylmethyl, pyrrolinylmethyl, etc.);
R⁵ is H, halogen, alkyl having 1 to 4 carbon atoms, halogenated alkyl having 1 to 4 carbon atoms, alkoxy having 1 to 4 carbon atoms, or halogenated alkoxy having 1 to 4 carbon atoms;
R⁶ is H or alkyl having 1 to 4 carbon atoms, which is branched or unbranched and which is unsubstituted or substituted one or more times by halogen (e.g., CH₃, CHF₂, CF₃, etc.);
R⁷ is H or alkyl having 1 to 12, preferably 1 to 8 carbon atoms, which is branched or unbranched and which is unsubstituted or substituted one or more times by halogen, hydroxy, cyano, C₁₋₄-alkoxy, oxo or combinations thereof, and wherein optionally one or more —CH₂CH₂— groups is replaced in each case by —CH═CH— or —C□C— (e.g., CH₃, CHF₂, CF₃, methoxyethyl, etc.),
   cycloalkyl having 3 to 10, preferably 3 to 8 carbon atoms, which is unsubstituted or substituted one or more times by halogen, hydroxy, oxo, cyano, alkyl having 1 to 4 carbon atoms, alkoxy having 1 to 4 carbon atoms, or combinations thereof (e.g., cyclopentyl),
   cycloalkylalkyl having 4 to 16, preferably 4 to 12 carbon atoms, which is unsubstituted or substituted in the cycloalkyl portion and/or the alkyl portion one or more times by halogen, oxo, cyano, hydroxy, C₁₋₄-alkyl, C₁₋₄-alkoxy or combinations thereof (e.g., cyclopentylmethyl, cyclopropylmethyl, etc.),
   aryl having 6 to 14 carbon atoms, which is unsubstituted or substituted one or more times by halogen, CF₃, OCF₃, alkyl, hydroxy, alkoxy, nitro, methylenedioxy, ethylenedioxy, cyano, or combinations thereof (e.g., methylphenyl, methoxyphenyl, chlorophenyl, etc.),
   arylalkyl in which the aryl portion has 6 to 14 carbon atoms and the alkyl portion, which is branched or unbranched, has 1 to 5 carbon atoms, wherein the arylalkyl radical is unsubstituted, substituted in the aryl portion one or more times by halogen, CF₃, OCF₃, alkyl, hydroxy, alkoxy, nitro, cyano, methylenedioxy, ethylenedioxy, or combinations thereof and/or substituted in the alkyl portion one or more times by halogen, oxo, hydroxy, cyano, or combinations thereof, and wherein in the alkyl portion one or more —CH₂CH₂— groups are each optionally replaced by CH═CH— or —C≡C—, and one or more —CH₂— groups are each optionally replaced by —O— or —NH— (e.g., phenylethyl, phenylpropyl, phenylbutyl, methoxyphenylethyl, methoxyphenylpropyl, chlorophenylethyl, chlorophenylpropyl, phenylethenyl, phenoxyethyl, phenoxybutyl, chlorophenoxyethyl, chlorophenylaminoethyl, etc.),
   a partially unsaturated carbocyclic group having 5 to 14 carbon atoms, which is unsubstituted or substituted one or more times by halogen, alkyl, alkoxy, hydroxy, nitro, cyano, oxo, or combinations thereof (e.g., cyclohexenyl, cyclohexadienyl, indanyl, tetrahydronaphthenyl, etc.),
   a heterocyclic group, which is saturated, partially saturated or unsaturated (e.g., heteroaryl), having 5 to 10 ring atoms in which at least 1 ring atom is an N, O or S atom, which is unsubstituted or substituted one or more times by halogen, hydroxy, aryl, alkyl, alkoxy, cyano, trifluoromethyl, nitro, oxo, or combinations thereof (e.g., 3-thienyl, 3-tetrahydrofuranyl, 3-pyrrolyl, etc.), or
   a heterocycle-alkyl group, wherein the heterocyclic portion is saturated, partially saturated or unsaturated (e.g., heteroaryl), and has 5 to 10 ring atoms in which at least 1 ring atom is an N, O or S atom, and the alkyl portion is branched or unbranched and has 1 to 5 carbon atoms, the heterocycle-alkyl group is unsubstituted, substituted one or more times in the heterocyclic portion by halogen, OCF₃, hydroxy, aryl, alkyl, alkoxy, cyano, trifluoromethyl, nitro, oxo, or combinations thereof, and/or substituted in the alkyl portion one or more times by halogen, oxo, hydroxy, cyano, or combinations thereof, and wherein in the alkyl portion one or more —CH₂CH₂— groups are each optionally replaced by —CH═CH— or —C≡C—, and one or more —CH₂— groups are each optionally replaced by —O— or —NH— (e.g., pyridylethyl, pydridylpropyl, methylpipetazinylethyl, etc.);
R⁸ is H,
   alkyl having 1 to 8, preferably 1 to 4 carbon atoms, which is unsubstituted or substituted one or more times by halogen, C₁₋₄-alkyl, C₁₋₄-alkoxy, oxo, or combinations thereof (e.g., methyl, ethyl, propyl, etc.),
   alkyl amino or dialkylamino wherein each alkyl portion has independently 1 to 8, preferably 1 to 4 carbon atoms (e.g., dimethylamo, etc.),
   a partially unsaturated carbocycle-alkyl group wherein the carbocyclic portion has 5 to 14 carbon atoms and the alkyl portion has 1 to 5 carbon atoms, and which is unsubstituted or substituted, preferably in the carbocyclic portion, one or more times by halogen, alkyl, alkoxy, nitro, cyano, oxo, or combinations thereof (e.g., cyclohexenylmethyl, etc.),
   cycloalkyl having 3 to 10, preferably 3 to 8 carbon atoms, which is unsubstituted or substituted one or more times by halogen, hydroxy, oxo, cyano, alkoxy, alkyl having 1 to 4 carbon atoms, or combinations thereof (e.g., cyclopentyl),
   cycloalkylalkyl having 4 to 16, preferably 4 to 12 carbon atoms, which is unsubstituted or substituted in the cycloalkyl portion and/or the alkyl portion one or more times by halogen, oxo, cyano, hydroxy, alkyl, alkoxy or combinations thereof (e.g., cyclopentylmethyl, cyclopropylmethyl, etc.),
   aryl having 6 to 14 carbon atoms which is unsubstituted or substituted one or more times by halogen, alkyl, hydroxy, alkoxy, alkoxyalkoxy, nitro, methylenedioxy, ethylenedioxy, trifluoromethyl, amino, aminomethyl, aminoalkyl, aminoalkoxy, dialkylamino, hydroxyalkyl (e.g., hydroxymethyl), hydroxamic acid (—C(O)—NHOH), tetrazole-5-yl, hydroxyalkoxy, carboxy, alkoxycarbonyl (e.g., tert-butyloxycarbonyl, ethoxycarbonyl), cyano, acyl, alkylthio, alkylsulfinyl, alkylsulfonyl, phenoxy, cycloalkyl, aryl (e.g., phenyl, naphthyl, biphenyl), heteroaryl or combinations thereof (e.g., substituted or unsubstituted phenyl and naphthyl, methylphenyl, chlorophenyl, fluorophenyl, vinylphenyl, cyanophenyl, methylenedioxophenyl, ethylphenyl, dichlorophenyl, carboxyphenyl, ethoxycarbonylphenyl, dimethylphenyl, hydroxymethylphenyl, nitrophenyl, aminophenyl, etc.),
   arylalkyl having 7 to 19 carbon atoms, wherein the aryl portion has 6 to 14 carbon atoms and the alkyl portion, which is branched or unbranched, has 1 to 5 carbon atoms, wherein the arylalkyl radical is unsubstituted or substituted, in the aryl portion, one or more times by halogen, trifluoromethyl, CF₃O, nitro, amino, alkyl, alkoxy, amino, alkylamino, dialkylamino, or combinations thereof, and/or substituted in the alkyl portion by halogen, cyano, alkyl having 1 to 4 carbon atoms (e.g., methyl), or combinations thereof, wherein in the alkyl portion one or more —CH₂CH₂— groups are each optionally replaced by —CH═CH— or —C≡C—, and/or one or more —CH₂— groups are each optionally replaced by —O— or —NH— (e.g., benzyl, phenethyl, phenpropyl, methylbenzyl, methoxybenzyl, trifluoromethyl, benzyl, methylenedioxobenzyl, etc.),
   a heterocyclic group, which is saturated, partially saturated or unsaturated (e.g., heteroaryl), having 5 to 10 ring atoms in which at least 1 ring atom is an N, O or S atom, which is unsubstituted or substituted one or more times by halogen, alkyl, hydroxy, alkoxy, alkoxyalkoxy, nitro, methylenedioxy, ethylenedioxy, trifluoromethyl, amino, aminomethyl, aminoalkyl, aminoalkoxy, dialkylamino, hydroxyalkyl (e.g., hydroxymethyl), hydroxamic acid (—C(O)—NHOH), tetrazole-5-yl, hydroxyalkoxy, carboxy, alkoxycarbonyl (e.g., tert-butyloxycarbonyl, ethoxycarbonyl), cyano, acyl, alkylthio, alkylsulfinyl, alkylsulfonyl, phenoxy, cycloalkyl, aryl, heteroaryl or combinations thereof (e.g., pyridyl, thienyl, pyrazinyl, quinolinyl, isoquinolinyl, pyrimidinyl, imidazolyl, thiazolyl, etc.), or
   a heterocycle-alkyl group, wherein the heterocyclic portion is saturated, partially saturated or unsaturated (e.g., heteroaryl), and has 5 to 10 ring atoms in which at least 1 ring atom is an N, O or S atom, and the alkyl portion, which is branched or unbranched, has 1 to 5 carbon atoms, the heterocycle-alkyl group is unsubstituted, substituted one or more times in the heterocyclic portion by halogen, alkyl, alkoxy, cyano, trifluoromethyl, CF₃O, nitro, oxo, amino, alkylamino, dialkylamino, or combinations thereof and/or substituted one or more times in the alkyl portion by halogen, cyano, alkyl having 1 to 4 carbon atoms (e.g., methyl), or combinations thereof (e.g., pyridylmethyl, pyridylpropyl, methylpyridylmethyl, etc.);
L is a single bond or a divalent aliphatic radical having 1 to 8 carbon atoms wherein one or more —CH₂— groups are each optionally replaced by —O—, —S—, —SO—, —SO₂—, —NR⁹—, —SO₂NR⁹—, —NR⁹SO₂—, —CO—, —CO₂—, —NR⁹CO—, —CONR⁹—, —NHCONH—, —OCONH, —NHCOO—, —SCONH—, —SCSNH—, —NHCSNH—, —CONHSO₂— or —SO₂NHCO— (e.g., —O—, —CH₂—, —CO—, —CO—O—, —O—CO—, —CO—NH—, —NH—CO—, —CH₂CH₂CH₂—NH—CO—, —CH₂—CH₂—O—, —SO₂—NH—CH₂CH₂—O—, —O—CH₂CH₂—O—, —CH₂—NH—CO—, —CO—NH—CH₂—, —SO₂—NH—, —CH₂—NH—SO₂—, —CH₂CH₂CH₂—SO₂—NH—, —SO₂—, —CONHSO₂—, —SO₂NHCO—, etc.); and
R⁹ is H,
   alkyl having 1 to 8, preferably 1 to 4 carbon atoms, which is branched or unbranched and which is unsubstituted or substituted one or more times with halogen, C₁₋₄-alkyl, C₁₋₄-alkoxy, oxo, or combinations thereof (e.g., methyl, ethyl, propyl, etc.),
   arylalkyl having 7 to 19 carbon atoms, wherein the aryl portion has 6 to 14 carbon atoms and the alkyl portion, which is branched or unbranched, has 1 to 5 carbon atoms, wherein the arylalkyl radical is unsubstituted or substituted, in the aryl portion, one or more times by halogen, trifluoromethyl, CF₃O, nitro, amino, alkyl, alkoxy, amino, alkylamino, dialkylamino, or combinations thereof, and/or substituted in the alkyl portion by halogen, cyano, alkyl having 1 to 4 carbon atoms (e.g., methyl), or combinations thereof, wherein in the alkyl portion one or more —CH₂CH₂— groups are each optionally replaced by —CH═CH— or —C≡C—, and/or one or more —CH₂— groups are each optionally replaced by —O— or —NH— (e.g., benzyl, phenethyl, phenpropyl, methylbenzyl, methoxybenzyl, trifluoromethyl, benzyl, methylenedioxobenzyl, etc.), or
   aryl having 6 to 14 carbon atoms and which is unsubstituted or substituted one or more times by halogen, alkyl, hydroxy, alkoxy, alkoxyalkoxy, nitro, methylenedioxy, ethylenedioxy, trifluoromethyl, amino, aminomethyl, aminoalkyl, aminoalkoxy dialkylamino, hydroxyalkyl (e.g., hydroxymethyl), hydroxamic acid (—C(O)—NHOH), tetrazole-5-yl, hydroxyalkoxy, carboxy, alkoxycarbonyl (e.g., tert-butyloxycarbonyl, ethoxycarbonyl), cyano, acyl, alkylthio, alkylsulfinyl, alkylsulfonyl, or combinations thereof (e.g., substituted or unsubstituted phenyl and naphthyl, methylphenyl, chlorophenyl, fluorophenyl, vinylphenyl, cyanophenyl, methylenedioxophenyl, ethylphenyl, dichlorophenyl, carboxyphenyl, ethoxycarbonylphenyl, dimethylphenyl, hydroxymethylphenyl, nitrophenyl, aminophenyl, etc.); and
R¹⁰ is H or alkyl having 1 to 4 carbon atoms, which is branched or unbranched and which is unsubstituted or substituted one or more times by halogen (e.g., CH₃, CHF₂, CF₃, etc.);

and pharmaceutically acceptable salts or solvates (e.g., hydrates) thereof, or solvates of pharmaceutically acceptable salts thereof;

wherein if the compound exhibits chirality it can be in the form of a mixture of enantiomers such as a racemate or a mixture of diastereomers, or can be in the form of a single enantiomer or a single diastereomer;

with the proviso that when R³ is pyridinylmethyl, R⁴ is other than substituted or unsubstituted piperidinyl, substituted or unsubstituted phenyl, or cyclohexyl,

and wherein said compound is not:
4-[[[5-methoxy-6-[[tetrahydro-3-furanyl]oxy]-2-pyridinyl](3-pyridinylmethyl)amino]methyl]-1-piperidinecarboxylic acid, 1,2-dimethylethyl ester,
N-[5-methoxy-6-[[tetrahydro-3-furanyl]oxy]-2-pyridinyl]-N-(4-piperidinylmethyl)-3-pyridinemethanamine,
4-[[[3,5-bis(trifluoromethyl)phenyl]methyl][5-bromo-4(phenylmethoxy)-2-pyrimidinyl]amino]-2-ethyl-3,4-dihydro-6-methoxy-1,5-naphthydrine-1(2H)-carboxylic acid ethyl ester,
5-chloro-N-(3-chlorophenyl)-4,6-difluoro-N-(4-methoxybenzyl)pyrimidin-2-amine,

or a pharmaceutically acceptable salt thereof, or solvate thereof, or solvate of a pharmaceutically acceptable salt thereof.

According to a further aspect of the invention, in Formula I R¹ is alkyl having 1 to 4 carbon atoms, halogenated alkyl having 1 to 4 carbon atoms, OR⁶, COR⁶, CONR⁶R¹⁰, or NR⁶COR¹⁰; R² is alkyl having 1 to 4 carbon atoms, halogenated alkyl having 1 to 4 carbon atoms, OR⁷, COR⁶, CONR⁶R¹⁰, or NR⁶COR¹⁰; R⁵ is H, alkyl having 1 to 4 carbon atoms, halogenated alkyl having 1 to 4 carbon atoms, alkoxy having 1 to 4 carbon atoms, or halogenated alkoxy having 1 to 4 carbon atoms; and R⁴ is cycloalkyl, aryl, heteroaryl, or a heterocyclic group, which in each case is unsubstituted or substituted.

According to a further aspect of the invention, in Formula I R¹ is alkyl having 1 to 4 carbon atoms, OR⁶, COR⁶, CONR⁶R¹⁰, or NR⁶COR¹⁰; R² is alkyl having 1 to 4 carbon atoms, OR⁷, COR⁶, CONR⁶R¹⁰), or NR⁶COR¹⁰; R⁵ is H, alkyl having 1 to 4 carbon atoms, or alkoxy having 1 to 4 carbon atoms; and R⁴ is cycloalkyl, aryl, heteroaryl, or a heterocyclic group, which in each case is unsubstituted or substituted.

According to a further aspect of the invention, in Formula I, B is CR⁵, R¹ is alkyl having 1 to 4 carbon atoms, halogenated alkyl having 1 to 4 carbon atoms, OR⁶, COR⁶, CONR⁶R¹⁰, or NR⁶COR¹⁰; R² is alkyl having 1 to 4 carbon atoms, halogenated alkyl having 1 to 4 carbon atoms, OR⁷, COR⁶, CONR⁶R¹⁰, or NR⁶COR¹⁰; R⁵ is H, alkyl having 1 to 4 carbon atoms, halogenated alkyl having 1 to 4 carbon atoms, alkoxy having 1 to 4 carbon atoms, or halogenated alkoxy having 1 to 4 carbon atoms; and R⁴ is cycloalkyl, aryl, heteroaryl, or a heterocyclic group, which in each case is unsubstituted or substituted.

According to a further aspect of the invention, in Formula I, B is CR⁵, R¹ is alkyl having 1 to 4 carbon atoms, OR⁶, COR⁶, CONR⁶R¹⁰, or NR⁶COR¹⁰; R² is alkyl having 1 to 4 carbon atoms, OR⁷, COR⁶, CONR⁶R¹⁰, or NR⁶COR¹⁰; R⁵ is H, alkyl having 1 to 4 carbon atoms, or alkoxy having 1 to 4 carbon atoms; and R⁴ is cycloalkyl, aryl, heteroaryl, or a heterocyclic group, which in each case is unsubstituted or substituted.

According to a further aspect of the invention, in Formula I, D is CR⁵, R¹ is alkyl having 1 to 4 carbon atoms, halogenated alkyl having 1 to 4 carbon atoms, OR⁶, COR⁶, CONR⁶R¹⁰, or NR⁶COR¹⁰; R² is alkyl having 1 to 4 carbon atoms, halogenated alkyl having 1 to 4 carbon atoms, OR⁷, COR⁶, CONR⁶R¹⁰, or NR⁶COR¹⁰; R⁵ is H, alkyl having 1 to 4 carbon atoms, halogenated alkyl having 1 to 4 carbon atoms, alkoxy having 1 to 4 carbon atoms, or halogenated alkoxy having 1 to 4 carbon atoms; and R⁴ is cycloalkyl, aryl, heteroaryl, or a heterocyclic group, which in each case is unsubstituted or substituted.

According to a further aspect of the invention, in Formula I, D is CR⁵, R¹ is alkyl having 1 to 4 carbon atoms, OR⁶, COR⁶, CONR⁶R¹⁰, or NR⁶COR¹⁰; R² is alkyl having 1 to 4 carbon atoms, OR⁷, COR⁶, CONR⁶R¹⁰, or NR⁶COR¹⁰; R⁵ is H, alkyl having 1 to 4 carbon atoms, or alkoxy having 1 to 4 carbon atoms; and R⁴ is cycloalkyl, aryl, heteroaryl, or a heterocyclic group, which in each case is unsubstituted or substituted.

According to a further aspect of the invention, in Formula I, one of A, B, and D is N (e.g., A is N) and the others are CR⁵; R¹ is alkyl having 1 to 4 carbon atoms, halogenated alkyl having 1 to 4 carbon, OR⁶, COR⁶, CONR⁶R¹⁰, or NR⁶COR¹⁰; R² is alkyl having 1 to 4 carbon atoms, halogenated alkyl having 1 to 4 carbon atoms, OR⁷, COR⁶, CONR⁶R¹⁰, or NR⁶COR¹⁰, R⁵ is H, alkyl having 1 to 4 carbon atoms, halogenated alkyl having 1 to 4 carbon atoms, alkoxy having 1 to 4 carbon atoms, or halogenated alkoxy having 1 to 4 carbon atoms; and R⁴ is cycloalkyl, aryl, heteroaryl, or a heterocyclic group, which in each case is unsubstituted or substituted.

According to a further aspect of the invention, in Formula I, one of A, B, and D is N (e.g., A is N) and the others are CR⁵; R¹ is alkyl having 1 to 4 carbon atoms, OR⁶, COR⁶, CONR⁶R¹⁰, or NR⁶COR¹⁰; R² is alkyl having 1 to 4 carbon atoms, OR⁷, COR⁶, CONR⁶R¹⁰, or NR⁶COR¹⁰; R⁵ is H, alkyl having 1 to 4 carbon atoms, or alkoxy having 1 to 4 carbon atoms; and R⁴ is cycloalkyl, aryl, heteroaryl, or a heterocyclic group, which in each case is unsubstituted or substituted.

According to a further aspect of the invention, B in Formula I is CR⁵.

According to a further aspect of the invention, D in Formula I is CR⁵.

According to a further aspect of the invention, one of A, B, and D in Formula I is N (e.g., A is N) and the others are CR⁵.

According to a further aspect of the invention, one of A, B, and D in Formula I is N (e.g., A is N) and the others are CH.

According to a further aspect of the invention, R⁴ is cycloalkyl, aryl, heteroaryl, or a heterocyclic group, which in each case is unsubstituted or substituted.

According to a further aspect of the invention, R⁴ is aryl (e.g., phenyl) or a heterocyclic group (e.g., piperidinyl), which in each case is unsubstituted or substituted.

According to a further aspect of the invention, R³ is an arylalkyl or a heteroarylalkyl group, other than pyridinylmethyl, which in each case is unsubstituted or substituted.

According to a further aspect of the invention, R³ is benzyl, thiazolylmethyl, oxazolylmethyl, or pyrimidinylmethyl, which in each case is unsubstituted or substituted.

According to a further aspect of the invention, one of A, B, and D in Formula I is N (e.g., A is N) and the others are CR⁵ (e.g., CH); R¹ is OR⁶; R² is OR⁷; R³ is an arylalkyl or a heteroarylalkyl group, other than pyridinylmethyl, which is in each case unsubstituted or substituted (e.g., benzyl, fluorobenzyl, bromobenzyl, thiazolylmethyl, oxoazolymethyl, pyrimidinylmethyl); 14 is aryl (such as phenyl and carboxyphenyl) or a heterocyclic group (such as piperidinyl), which is in each case substituted or unsubstituted; R⁶ is alkyl (e.g., methyl), or halogenated alkyl (e.g., CF₃, CHF₂); and R⁷ is alkyl (such as methyl, ethyl, isopropyl), halogenated alkyl (e.g., CF₃, CHF₂), cycloalkyl (such as cyclopropyl, cyclobutyl, cyclopentyl), cycloalkylalkyl (such as cyclopropylmethyl), or a heterocyclic group (such as tetrahydrofuranyl); and wherein if R³ is prymidinylmethyl, then R⁴ is not piperidinyl, and preferably R⁴ is aryl.

In accordance with a further compound and/or method aspect of the invention, the compounds of Formula I are selected from the following subformulae:

Ia: A is N
B and D are each independently CH,
R¹ is OR⁶ wherein R⁶ is fluorinated alkyl (e.g., CF₃, CHF₂),
R² is OR⁷ wherein R⁷ is cycloalkylalkyl (e.g., cyclopropylmethyl),
R³ is heteroarylalkyl other than pyridinylmethyl (e.g., thiazolylmethyl), and
R⁴ is aryl which is unsubstituted or substituted (e.g., carboxyphenyl);

Ib: A is N
B and D are each independently CH,
R¹ is OR⁶ wherein R⁶ is fluorinated alkyl (e.g., CF₃, CHF₂),
R² is OR₇ wherein R⁷ is alkyl (e.g., methyl, ethyl, isopropyl),
R³ is heteroarylalkyl other than pyridinylmethyl (e.g., oxazolylmethyl, thiazolylmethyl, pyrimidinylmethyl), and
R⁴ is aryl which is unsubstituted or substituted (e.g., carboxyphenyl);

Ic: A is N
B and D are each independently CH,
R¹ is OR⁶ wherein R⁶ is alkyl (e.g., methyl),
R² is OR⁷ wherein R⁷ is cycloalkylalkyl (e.g., cyclopropylmethyl),
R³ is heteroarylalkyl other than pyridinylmethyl (e.g., thiazolylmethyl), or arylalkyl which is unsubstituted or substituted (e.g. benzyl, halo-substituted benzyl), and
R⁴ is aryl which is unsubstituted or substituted (e.g., carboxyphenyl) or a saturated or partially saturated heterocyclic group, which may be unsubstituted or substituted (e.g., piperidinyl);

Id: A is N
B and D are each independently CH,
R¹ is OR⁶ wherein R⁶ is alkyl (e.g., methyl),
R² is OR⁷ wherein R⁷ is cycloalkyl (e.g., cyclopentyl),
R³ is arylalkyl which is unsubstituted or substituted (e.g. benzyl), and
R⁴ is a saturated or partially saturated heterocyclic group, which may be unsubstituted or substituted (e.g., piperidinyl);

Ie: A is N
B and D are each independently CR⁵,
R¹ is OR⁶,
R² is OR⁷,
R³ is arylalkyl which is unsubstituted or substituted (e.g. benzyl), and
R⁴ is a saturated or partially saturated heterocyclic group, which may be unsubstituted or substituted (e.g., piperidinyl); and

If: A is N

B and D are each independently CH,

R⁴ is OR⁶ wherein R⁶ is alkyl (e.g., methyl),
R² is OR⁷ wherein R⁷ is alkyl (e.g., ethyl, isopropyl),
R³ is heteroarylalkyl other than pyridinylmethyl (e.g., thiazolylmethyl, oxazolylmethyl, pyrimidinylmethyl), or arylalkyl which is unsubstituted or substituted (e.g. benzyl, halogen-substituted benzyl, such as bromobenzyl, fluorobenzyl), and
R⁴ is aryl which is unsubstituted or substituted (e.g., carboxyphenyl) or a saturated or partially saturated heterocyclic group, which may be unsubstituted or substituted (e.g., piperidinyl);

According to a further compound and/or method aspect of the invention, the compounds of Formula I are selected from:
1) 4-[[6-(Cyclopropylmethoxy)-5-(difluoromethoxy)pyridin-2-yl](1,3-thiazol-5-ylmethyl)amino]benzoic acid,
2) 4-[[6-(Cyclopropylmethoxy)-5-methoxypyridin-2-yl](1,3-thiazol-5-ylmethyl)amino]benzoic acid trifluoroacetate,
3) N-Benzyl-6-(cyclopentyloxy)-5-methoxy-N-piperidin-4-ylpyridin-2-amine oxalate,
4) 6-Isopropoxy-5-methoxy-N-piperidin-4-yl-N-(1,3-thiazol-5-ylmethyl)pyridin-2-amine hydrochloride,
5) N-(4-Fluorobenzyl)-6-isopropoxy-5-methoxy-N-piperidin-4-ylpyridin-2-amine hydrochloride,
6) 4-[[5-(Difluoromethoxy)-6-ethoxypyridin-2-yl](1,3-oxazol-5-ylmethyl)amino]benzoic acid hydrochloride,
7) 4-[(6-Ethoxy-5-methoxypyridin-2-yl)(1,3-thiazol-5-ylmethyl)amino]benzoic acid hydrochloride,
8) 4-[(6-Ethoxy-5-methoxypyridin-2-yl)(1,3-oxazol-5-ylmethyl)amino]benzoic acid hydrochloride,
9) 4-[[5-(Difluoromethoxy)-6-ethoxypyridin-2-yl](pyrimidin-5-ylmethyl)amino]benzoic acid,
10) 4-[[5-(Difluoromethoxy)-6-ethoxypyridin-2-yl](1,3-thiazol-5-ylmethyl)amino]benzoic acid,

11) 4-[[5-(Difluoromethoxy)-6-methoxypyridin-2-yl](1,3-thiazol-5-ylmethyl)amino]benzoic acid,
12) N-(4-Bromobenzyl)-6-(cyclopropylmethoxy)-5-methoxy-N-piperidin-4-ylpyridin-2-amine,
13) 4-[(6-Ethoxy-5-methoxypyridin-2-yl)pyrimidin-5-ylmethyl)amino]benzoic acid, and
14) 3-[(6-Ethoxy-5-methoxypyridin-2-yl)(pyrimidin-5-ylmethyl)amino]benzoic acid,
   and pharmaceutically acceptable salts thereof,
   wherein a compound listed above (in either a free base form or in the form of a pharmaceutically acceptable salt thereof) can also be in the form of a solvate (such as a hydrate),
   wherein a compound listed above (in a free base form or solvate thereof, or in the form of a pharmaceutically acceptable salt or solvate thereof) can also be in the form of a polymorph, and
   wherein if the compound exhibits chirality it can be in the form of a mixture of enantiomers such as a racemate or a mixture of diastereomers, or can be in the form of a single enantiomer or a single diastereomer.

The following table presents structures for several compounds of Formula I in accordance with the present invention:

| | |
|---|---|
| Name | Structure |

| | | |
|---|---|---|
| 1 | 4-[[6-(cyclopropylmethoxy)-5- (difluoromethoxy)pyridin-2-yl](1,3- thiazol-5-ylmethyl)amino]benzoic acid | 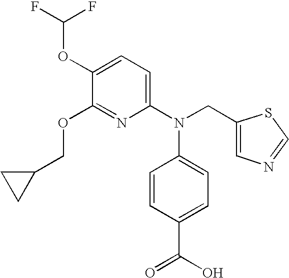 |
| 2 | 4-[[6-(cyclopropylmethoxy)-5- methoxypyridin-2-yl](1,3- thiazol-5-ylmethyl)amino]benzoic acid trifluoroacetate | 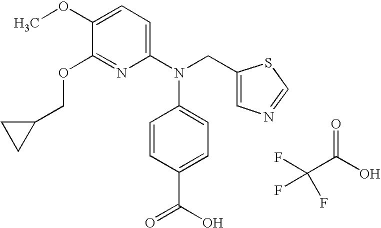 |
| 3 | N-benzyl-6-(cyclopentyloxy)-5-methoxy- N-piperidin-4-ylpyridin-2-amine oxalate | 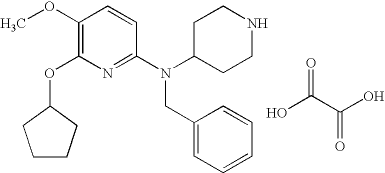 |
| 4 | 6-isopropoxy-5-methoxy-N-piperidin-4-yl- N-(1,3-thiazol-5-ylmethyl)pyridin-2-amine hydrochloride | 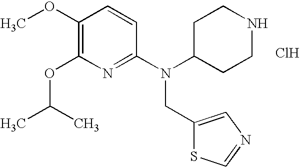 |
| 5 | N-(4-fluorobenzyl)-6-isopropoxy-5- methoxy-N-piperidin-4-ylpyridin-2-amine hydrochloride | 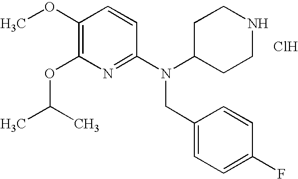 |
| 6 | 4-[[5-(difluoromethoxy)-6-ethoxypyridin- 2-yl](1,3-oxazol-5- ylmethyl)amino]benzoic acid hydrochloride | 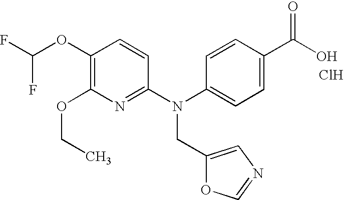 |
| 7 | 4-[(6-ethoxy)-5-methoxypyridin-2-yl)(1,3- thiazol-5-ylmethyl)amino]benzoic acid hydrochloride | 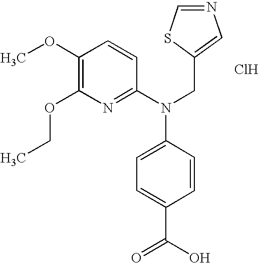 |
| 8 | 4-[(6-ethoxy-5-methoxypyridin-2-yl)(1,3- oxazol-5-ylmethyl)amino]benzoic acid hydrochloride | 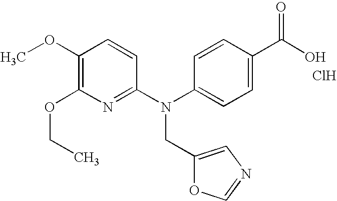 |
| 9 | 4-[[5-(difluoromethoxy)-6-ethoxypyridin- 2-yl](pyrimidin-5-ylmethyl)amino]benzoic acid | 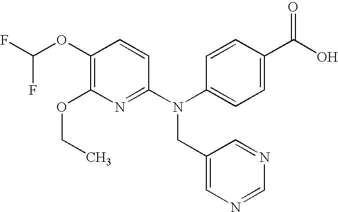 |
| 10 | 4-[[5-(difluoromethoxy)-6-ethoxypyridin- 2-yl](1,3-thiazol-5- ylmethyl)amino]benzoic acid | 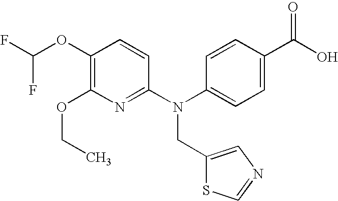 |
| 11 | 4-[[5-(difluoromethoxy)-6- methoxypyridin-2-yl](1,3-thiazol-5- ylmethyl)amino]benzoic acid | 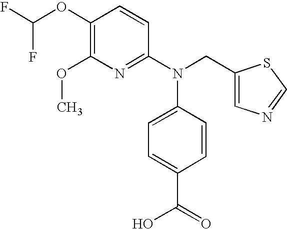 |
| 12 | N-(4-bromobenzyl)-6- (cyclopropylmethoxy)-5-methoxy-N- piperidin-4-ylpyridin-2-amine | 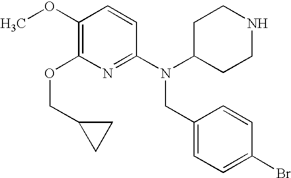 |
| 13 | 4-[(6-ethoxy-5-methoxypyridin-2- yl)(pyrimidin-5-ylmethyl)amino]benzoic acid | 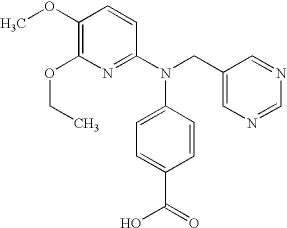 |
| 14 | 3-[(6-ethoxy-5-methoxypyridin-2- yl)(pyrimidin-5-ylmethyl)amino]benzoic acid | 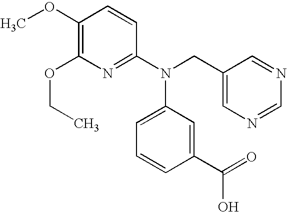 |

The present invention includes compounds of Formula II:

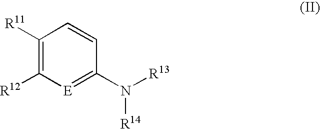

wherein

E is N or CR¹⁵;

R¹¹ is halogen, alkyl having 1 to 4 carbon atoms (e.g., methyl, ethyl), halogenated alkyl having 1 to 4 carbon atoms (e.g., CH₂F, CHE₂, CF₃), OR¹⁶, COR¹⁶, CONHR¹⁶R²⁰, or NR¹⁶COR²⁰; R¹² is halogen, alkyl having 1 to 4 carbon atoms (e.g., methyl, ethyl), halogenated alkyl having 1 to 4 carbon atoms (e.g., CH₂F, CHF₂, CF₃), OR¹⁷, COR¹⁶, CONHR¹⁶R²⁰, or NR¹⁶COR²⁰,
R¹³ a non-aromatic heterocyclic group, which is fully saturated or partially saturated, having 5 to 10 ring atoms in which at least 1 ring atom is an N, O or S atom, which is unsubstituted or substituted one or more times by halogen, alkyl, hydroxy, alkoxy, alkoxyalkoxy, hydroxyalkyl-alkoxy, dihydroxyalkyl-alkoxy, nitro, oxo, methylenedioxy, ethylenedioxy, trifluoromethyl, OCF₃, amino, aminomethyl, aminoalkyl, aminoalkoxy, dialkylamino, hydroxyalkyl (e.g., hydroxymethyl), dihydroxyalkyl, hydroxamic acid (—C(O)—NHOH), tetrazole-5-yl, hydroxyalkoxy, carboxy, alkoxycarbonyl (e.g., tert-butyloxycarbonyl, ethoxycarbonyl), cyano, acyl (e.g., optionally substituted acetyl or optionally substituted benzoyl), alkylthio, alkylsulfinyl, alkylsulfonyl, phenylsulfonyl, phenoxy, cycloalkyl, aryl, heteroaryl or combinations thereof (e.g., piperidinyl, pyrrolydinyl, morpholinyl, piperazinyl etc.),
R¹⁴ is cycloalkyl having 3 to 10, preferably 3 to 8 carbon atoms, which is unsubstituted or substituted one or more times by halogen, hydroxy, oxo, cyano, alkyl having 1 to 4 carbon atoms, alkoxy having 1 to 4 carbon atoms, or combinations thereof (e.g., cyclopentyl),
   aryl having 6 to 14 carbon atoms and which is unsubstituted or substituted one or more times by halogen, alkyl, alkenyl, alkynyl, hydroxy, alkoxy, alkoxyalkoxy, nitro, methylenedioxy, ethylenedioxy, trifluoromethyl, OCF₃, amino, aminoalkyl, aminoalkoxy, dialkylamino, amido (e.g., CONH₂), hydroxyalkyl (e.g., hydroxymethyl), hydroxamic acid (—C(O)—NHOH), pyrrolyl, tetrazole-5-yl, 2(-heterocycle)tetrazole-5-yl (e.g., 2-(2-tetrahydropyranyl)tetrazole-5-yl), hydroxyalkoxy, carboxy, carboxyalkyl, alkoxycarbonyl (e.g., tert-butyloxycarbonyl, ethoxycarbonyl), cyano, acyl, alkylthio, alkylsulfinyl, alkylsulfonyl, phenoxy, trialkylsilyloxy (e.g. tert-butyldimethylsilyloxy), R¹⁸-M-, or combinations thereof (e.g., substituted or unsubstituted phenyl, naphthyl, and biphenyl, such as, but not limited to, phenyl, methylphenyl, chlorophenyl, fluorophenyl, methoxyphenyl, vinylphenyl, cyanophenyl, methylenedioxophenyl, ethylphenyl, dichlorophenyl, carboxyphenyl, ethoxycarbonylphenyl, dimethylphenyl, hydroxymethylphenyl, nitrophenyl, aminophenyl, dimethylaminophenyl, amidophenyl, etc.),
   heteroaryl having 5 to 10 ring atoms in which at least 1 ring atom is a heteroatom (e.g., N, S or O), which is unsubstituted or substituted one or more times by halogen, alkyl, hydroxy, alkoxy, alkoxyalkoxy, nitro, methylenedioxy, ethylenedioxy, trifluoromethyl, amino, aminomethyl, aminoalkyl, aminoalkoxy, dialkylamino, hydroxyalkyl (e.g., hydroxymethyl), hydroxamic acid (—C(O)—NHOH), tetrazole-5-yl, hydroxyalkoxy, carboxy, carboxyalkyl, alkoxycarbonyl (e.g., tert-butyloxycarbonyl, ethoxycarbonyl), cyano, acyl, alkylthio, alkylsulfinyl, alkylsulfonyl, phenoxy, trialkylsilyloxy (e.g. tert-butyldimethylsilyloxy), R¹⁸-M-, or combinations thereof (e.g., pyridyl, thienyl, pyrazinyl, quinolinyl, isoquinolinyl, pyrimidinyl, imidazolyl, thiazolyl, etc.),
   a heterocyclic group, which is saturated or partially saturated, having 5 to 10 ring atoms in which at least 1 ring atom is an N, O or S atom, which is unsubstituted or substituted one or more times by halogen, alkyl, hydroxy, alkoxy, alkoxyalkoxy, nitro, oxo, methylenedioxy, ethylenedioxy, trifluoromethyl, OCF₃, amino, aminomethyl, aminoalkyl, aminoalkoxy, dialkylamino, hydroxyalkyl (e.g., hydroxymethyl), hydroxaric acid (—C(O)—NHOH), tetrazole-5-yl, hydroxyalkoxy, carboxy, alkoxycarbonyl (e.g., tert-butyloxycarbonyl, ethoxycarbonyl), cyano, acyl (e.g., optionally substituted acetyl or optionally substituted benzoyl), alkylthio, alkylsulfinyl, alkylsulfonyl, phenylsulfonyl, phenoxy, cycloalkyl, aryl, heteroaryl or combinations thereof (e.g., piperidinyl, pyrrolydinyl, amidazolidinyl, pyrrolinyl, etc.), and
   a heterocycle-alkyl group, wherein the heterocyclic portion is saturated, partially saturated or unsaturated (e.g., heteroaryl), and has 5 to 10 ring atoms in which at least 1 ring atom is an N, O or S atom, and the alkyl portion is branched or unbranched and has 1 to 5 carbon atoms, the heterocycle-alkyl group is unsubstituted, substituted one or more times in the heterocyclic portion by halogen, alkyl, hydroxy, alkoxy, alkoxyalkoxy, nitro, oxo, methylenedioxy, ethylenedioxy, trifluoromethyl, OCF₃, amino, aminomethyl, aminoalkyl, aminoalkoxy, dialkylamino, hydroxyalkyl (e.g., hydroxymethyl), hydroxamic acid (—C(O)—NHOH), tetrazole-5-yl, hydroxyalkoxy, carboxy, alkoxycarbonyl (e.g., tert-butyloxycarbonyl, ethoxycarbonyl), cyano, acyl alkylthio, alkylsulfinyl, alkylsulfonyl, phenylsulfonyl, phenoxy, cycloalkyl, aryl, heteroaryl or combinations thereof, and/or substituted in the alkyl portion one or more times by halogen, oxo, hydroxy, cyano, or combinations thereof, and wherein in the alkyl portion one or more —CH₂CH₂— groups are each optionally replaced by —CH═CH— or —C≡C—, and one or more —CH₂— groups are each optionally replaced by —O— or —NH— (e.g., pyridylethyl, pydridylpropyl, methylpiperazinylethyl, piperidinylmethyl, pyrrolydinylmethyl, amidazolidinylmethyl, pyrrolinylmethyl, etc.);
R¹⁵ is H, halogen, alkyl having 1 to 4 carbon atoms, halogenated alkyl having 1 to 4 carbon atoms, alkoxy having 1 to 4 carbon atoms, or halogenated alkoxy having 1 to 4 carbon atoms;
R¹⁶ is H or alkyl having 1 to 4 carbon atoms, which is branched or unbranched and which is unsubstituted or substituted one or more times by halogen (e.g., CH₃, CHF₂, CF₃, etc.);
R¹⁷ is H or alkyl having 1 to 12, preferably 1 to 8 carbon atoms, which is branched or unbranched and which is unsubstituted or substituted one or more times by halogen, hydroxy, cyano, C₁₋₄-alkoxy, oxo or combinations thereof, and wherein optionally one or more —CH₂CH₂— groups is replaced in each case by —CH═CH— or —C≡C— (e.g., CH₃, CHF₂, CF₃, methoxyethyl, etc.),
   cycloalkyl having 3 to 10, preferably 3 to 8 carbon atoms, which is unsubstituted or substituted one or more times by halogen, hydroxy, oxo, cyano, alkyl having 1 to 4 carbon atoms, alkoxy having 1 to 4 carbon atoms, or combinations thereof (e.g., cyclopentyl),
   cycloalkylalkyl having 4 to 16, preferably 4 to 12 carbon atoms, which is unsubstituted or substituted in the cycloalkyl portion and/or the alkyl portion one or more times by halogen, oxo, cyano, hydroxy, C₁₋₄-alkyl, C₁₋₄-alkoxy or combinations thereof (e.g., cyclopentylmethyl, cyclopropylmethyl, etc.),
   aryl having 6 to 14 carbon atoms, which is unsubstituted or substituted one or more times by halogen, CF₃, OCF₃, alkyl, hydroxy, alkoxy, nitro, methylenedioxy, ethylenedioxy, cyano, or combinations thereof (e.g., methylphenyl, methoxyphenyl, chlorophenyl, etc.),
   arylalkyl in which the aryl portion has 6 to 14 carbon atoms and the alkyl portion, which is branched or unbranched, has 1 to 5 carbon atoms, wherein the arylalkyl radical is unsubstituted, substituted in the aryl portion one or more times by halogen, CF₃, OCF₃, alkyl, hydroxy, alkoxy, nitro, cyano, methylenedioxy, ethylenedioxy, or combinations thereof, and/or substituted in the alkyl portion one or more times by halogen, oxo, hydroxy, cyano, or combinations thereof, and wherein in the alkyl portion one or more —CH₂CH₂— groups are each optionally replaced by —CH═CH— or —C≡C—, and one or more —CH₂— groups are each optionally replaced by —O— or —NH— (e.g., phenylethyl, phenylpropyl, phenylbutyl, methoxyphenylethyl, methoxyphenylpropyl, chlorophenylethyl, chlorophenylpropyl, phenylethenyl, phenoxyethyl, phenoxybutyl, chlorophenoxyethyl, chlorophenylaminoethyl, etc.),
   a partially unsaturated carbocyclic group having 5 to 14 carbon atoms, which is unsubstituted or substituted one or more times by halogen, alkyl, alkoxy, hydroxy, nitro, cyano, oxo, or combinations thereof (e.g., cyclohexenyl, cyclohexadienyl, indanyl, tetrahydronaphthenyl, etc.),
   a heterocyclic group, which is saturated, partially saturated or unsaturated (e.g., heteroaryl), having 5 to 10 ring atoms in which at least 1 ring atom is an N, O or S atom, which is unsubstituted or substituted one or more times by halogen, hydroxy, aryl, alkyl, alkoxy, cyano, trifluoromethyl, nitro, oxo, or combinations thereof (e.g., 3-thienyl, 3-tetrahydrofuranyl, 3-pyrrolyl, etc.), or
   a heterocycle-alkyl group, wherein the heterocyclic portion is saturated, partially saturated or unsaturated (e.g., heteroaryl), and has 5 to 10 ring atoms in which at least 1 ring atom is an N, O or S atom, and the alkyl portion is branched or unbranched and has 1 to 5 carbon atoms, the heterocycle-alkyl group is unsubstituted, substituted one or more times in the heterocyclic portion by halogen, OCF₃, hydroxy, aryl, alkyl, alkoxy, cyano, trifluoromethyl, nitro, oxo, or combinations thereof, and/or substituted in the alkyl portion one or more times by halogen, oxo, hydroxy, cyano, or combinations thereof, and wherein in the alkyl portion one or more —CH₂CH₂— groups are each optionally replaced by —CH═CH— or —CH—C—, and one or more —CH₂— groups are each optionally replaced by —O— or —NH— (e.g., pyridylethyl, pydridylpropyl, methylpiperazinylethyl, etc.);
R¹⁸ is H,
   alkyl having 1 to 8, preferably 1 to 4 carbon atoms, which is unsubstituted or substituted one or more times by halogen, C₁₋₄-alkyl, C₁₋₄-alkoxy, oxo, or combinations thereof (e.g., methyl, ethyl, propyl, etc.),
   alkylamino or dialkylamino wherein each alkyl portion has independently 1 to 8, preferably 1 to 4 carbon atoms (e.g., dimethylamino, etc.),
   a partially unsaturated carbocycle-alkyl group wherein the carbocyclic portion has 5 to 14 carbon atoms and the alkyl portion has 1 to 5 carbon atoms, and which is unsubstituted or substituted, preferably in the carbocyclic portion, one or more times by halogen, alkyl, alkoxy, nitro, cyano, oxo, or combinations thereof (e.g., cyclohexenylmethyl, etc.),
   cycloalkyl having 3 to 10, preferably 3 to 8 carbon atoms, which is unsubstituted or substituted one or more times by halogen, hydroxy, oxo, cyano, alkoxy, alkyl having 1 to 4 carbon atoms, or combinations thereof (e.g., cyclopentyl),
   cycloalkylalkyl having 4 to 16, preferably 4 to 12 carbon atoms, which is unsubstituted or substituted in the cycloalkyl portion and/or the alkyl portion one or more times by halogen, oxo, cyano, hydroxy, alkyl, alkoxy or combinations thereof (e.g., cyclopentylmethyl, cyclopropylmethyl, etc.),
   aryl having 6 to 14 carbon atoms which is unsubstituted or substituted one or more times by halogen, alkyl, hydroxy, alkoxy, alkoxyalkoxy, nitro, methylenedioxy, ethylenedioxy, trifluoromethyl, amino, aminomethyl, aminoalkyl, aminoalkoxy, dialkylamino, hydroxyalkyl (e.g., hydroxymethyl), hydroxamic acid (—C(O)—NHOH), tetrazole-5-yl, hydroxyalkoxy, carboxy, alkoxycarbonyl (e.g., tert-butyloxycarbonyl, ethoxycarbonyl), cyano, acyl, alkylthio, alkylsulfinyl, alkylsulfonyl, phenoxy, cycloalkyl, aryl (e.g., phenyl, naphthyl, biphenyl), heteroaryl or combinations thereof (e.g., substituted or unsubstituted phenyl and naphthyl, methylphenyl, chlorophenyl, fluorophenyl, vinylphenyl, cyanophenyl, methylenedioxophenyl, ethylphenyl, dichlorophenyl, carboxyphenyl, ethoxycarbonylphenyl, dimethylphenyl, hydroxymethylphenyl, nitrophenyl, aminophenyl, etc.),
   arylalkyl having 7 to 19 carbon atoms, wherein the aryl portion has 6 to 14 carbon atoms and the alkyl portion, which is branched or unbranched, has 1 to 5 carbon atoms, wherein the arylalkyl radical is unsubstituted or substituted, in the aryl portion, one or more times by halogen, trifluoromethyl, CF₃O, nitro, amino, alkyl, alkoxy, amino, alkylamino, dialkylamino, or combinations thereof, and/or substituted in the alkyl portion by halogen, cyano, alkyl having 1 to 4 carbon atoms (e.g., methyl), or combinations thereof, wherein in the alkyl portion one or more —CH₂CH₂— groups are each optionally replaced by —CH═CH— or —C≡C—, and/or one or more —CH₂— groups are each optionally replaced by —O— or —NH— (e.g., benzyl, phenethyl, phenpropyl, methylbenzyl, methoxybenzyl, trifluoromethylbenzyl, methylenedioxobenzyl, etc.),
   a heterocyclic group, which is saturated, partially saturated or unsaturated (e.g., heteroaryl), having 5 to 10 ring atoms in which at least 1 ring atom is an N, O or S atom, which is unsubstituted or substituted one or more times by halogen, alkyl, hydroxy, alkoxy, alkoxyalkoxy, nitro, methylenedioxy, ethylenedioxy, trifluoromethyl, amino, aminomethyl, aminoalkyl, aminoalkoxy, dialkylamino, hydroxyalkyl (e.g., hydroxymethyl), hydroxamic acid (—C(O)—NHOH), tetrazole-5-yl, hydroxyalkoxy, carboxy, alkoxycarbonyl (e.g., tert-butyloxycarbonyl, ethoxycarbonyl), cyano, acyl, alkylthio, alkylsulfinyl, alkylsulfonyl, phenoxy, cycloalkyl, aryl, heteroaryl or combinations thereof (e.g., pyridyl, thienyl, pyrazinyl, quinolinyl, isoquinolinyl, pyrimidinyl, imidazolyl, thiazolyl, etc.), or
   a heterocycle-alkyl group, wherein the heterocyclic portion is saturated, partially saturated or unsaturated (e.g., heteroaryl), and has 5 to 10 ring atoms in which at least 1 ring atom is an N, O or S atom, and the alkyl portion, which is branched or unbranched, has 1 to 5 carbon atoms, the heterocycle-alkyl group is unsubstituted, substituted one or more times in the heterocyclic portion by halogen, alkyl, alkoxy, cyano, trifluoromethyl, CF₃O, nitro, oxo, amino, alkylamino, dialkylamino, or combinations thereof and/or substituted one or more times in the alkyl portion by halogen, cyano, alkyl having 1 to 4 carbon atoms (e.g., methyl), or combinations thereof (e.g., pyridylmethyl, pyridylpropyl, methylpyridylmethyl, etc.);
M is a single bond or a divalent aliphatic radical having 1 to 8 carbon atoms wherein one or more —CH₂— groups are each optionally replaced by —O—, —S—, —SO—, —SO₂—, —NR¹⁹—, —SO₂NR¹⁹—, —NR¹⁵SO₂—, —CO—, —CO₂—, —NR¹⁹CO—, —CONR¹⁹—, —NHCONH—, —OCONH, —NHCOO—, —SCONH—, —SCSNH—, —NHCSNH—, —CONHSO₂— or —SO₂NHCO— (e.g., —O—, —CH₂—, —CO—, —CO—O—, —O—CO—, —CO—NH—, —NH—CO—, —2.8 CH₂CH₂CH₂—NH—CO—, —CH₂—CH₂—O—, —SO₂—NH—CH₂CH₂—O—, —O—CH₂CH₂—O—, —CH₂—NH—CO—, —CO—NH—CH₂—, —SO₂—NH—, —CH₂—NH—SO₂—, —CH₂CH₂CH₂—SO₂—NH—, —SO₂—, —CONHSO₂—, —SO₂NHCO—, etc.); and
R¹⁹ is H,
   alkyl having 1 to 8, preferably 1 to 4 carbon atoms, which is branched or unbranched and which is unsubstituted or substituted one or more times with halogen, C₁₋₄-alkyl, C₁₋₄-alkoxy, oxo, or combinations thereof (e.g., methyl, ethyl, propyl, etc.),
   arylalkyl having 7 to 19 carbon atoms, wherein the aryl portion has 6 to 14 carbon atoms and the alkyl portion, which is branched or unbranched, has 1 to 5 carbon atoms, wherein the arylalkyl radical is unsubstituted or substituted, in the aryl portion, one or more times by halogen, trifluoromethyl, CF₃O, nitro, amino, alkyl, alkoxy, amino, alkylamino, dialkylamino, or combinations thereof, and/or substituted in the alkyl portion by halogen, cyano, alkyl having 1 to 4 carbon atoms (e.g., methyl), or combinations thereof, wherein in the alkyl portion one or more —CH₂CH₂— groups are each optionally replaced by —CH═CH— or —C≡C—, and/or one or more —CH₂— groups are each optionally replaced by —O— or —NH— (e.g., benzyl, phenethyl, phenpropyl, methylbenzyl, methoxybenzyl, trifluoromethyl, benzyl, methylenedioxobenzyl, etc.), or
   aryl having 6 to 14 carbon atoms and which is unsubstituted or substituted one or more times by halogen, alkyl, hydroxy, alkoxy, alkoxyalkoxy, nitro, methylenedioxy, ethylenedioxy, trifluoromethyl, amino, aminomethyl, aminoalkyl, aminoalkoxy dialkylamino, hydroxyalkyl (e.g., hydroxymethyl), hydroxamic acid (—C(O)—NHOH), tetrazole-5-yl, hydroxyalkoxy, carboxy, alkoxycarbonyl (e.g., tert-butyloxycarbonyl, ethoxycarbonyl), cyano, acyl, alkylthio, alkylsulfinyl, alkylsulfonyl, or combinations thereof (e.g., substituted or unsubstituted phenyl and naphthyl, methylphenyl, chlorophenyl, fluorophenyl, vinylphenyl, cyanophenyl, methylenedioxophenyl, ethylphenyl, dichlorophenyl, carboxyphenyl, ethoxycarbonylphenyl, dimethylphenyl, hydroxymethylphenyl, nitrophenyl, aminophenyl, etc.); and
R²⁰ is H or alkyl having 1 to 4 carbon atoms, which is branched or unbranched and which is unsubstituted or substituted one or more times by halogen (e.g., CH₃, CHF₂, CF₃, etc.); and

and pharmaceutically acceptable salts or solvates (e.g., hydrates) thereof, or solvates of pharmaceutically acceptable salts thereof;

wherein if the compound exhibits chirality it can be in the form of a mixture of enantiomers such as a racemate or a mixture of diastereomers, or can be in the form of a single enantiomer or a single diastereomer;
wherein when R¹⁴ is pyridinylmethyl, R¹³ is other than unsubstituted or substituted piperidinyl, and

with the proviso that said compound is not:
6-[(3,4-dimethoxyphenyl)(2-furanylmethyl)amino]-1,2,4-triazine-3,5(2H,4H)dione,
N-(6-(cyclopentyloxy)-5-methoxy-2-pyridinyl]-N-4-piperidinyl-5-pyrimidinemethanamine,
N,N-dibutyl-4-[(3-chloro-4-methoxyphenyl)(1-propyl-4-piperidinyl)amino]benzamide,
4-[(3-chloro-4-methoxyphenyl)(1-propyl-4-piperidinyl)amino]-N,N-bis(1-methylethyl)benzamide,
4-[(3-chloro-4-methoxyphenyl)(1-propyl-4-piperidinyl)amino]-N,N-dipropylbenzamide,
4-[(3-chloro-4-methoxyphenyl)(1-propyl-4-piperidinyl)amino]-N,N-diethylbenzamide,
N-(3,4-dimethylphenyl)-N-4-morpholinyl-4-morpholinamine,
N-(3,4-dimethylphenyl)-N-1-piperidinyl-1-piperidinamine, or
6-(cyclopentyloxy)-5-methoxy-N-phenyl-N-piperidin-4-ylpyridin-2-amine,

or a pharmaceutically acceptable salt thereof, or solvate thereof, or solvate of a pharmaceutically acceptable salt thereof.

According to a further aspect of the invention, in Formula II, R¹⁷ is H, alkyl which is unsubstituted or substituted, cycloalkylalkyl which is unsubstituted or substituted, aryl which is unsubstituted or substituted, arylalkyl which is unsubstituted or substituted, a partially unsaturated carbocyclic group which is unsubstituted or substituted, a heterocyclic group which is unsubstituted or substituted, or a heterocycle-alkyl group which is unsubstituted or substituted; and R¹⁴ is cycloalkyl which is unsubstituted or substituted, aryl which is unsubstituted or substituted one or more times by halogen, alkyl, alkenyl, alkynyl, hydroxy, alkoxy, alkoxyalkoxy, nitro, methylenedioxy, ethylenedioxy, trifluoromethyl, OCF₃, amino, aminoalkyl, aminoalkoxy, dialkylamino, hydroxyalkyl, hydroxamic acid, hydroxyalkoxy, carboxy, carboxyalkyl, alkoxycarbonyl, cyano, acyl, alkylthio, alkylsulfinyl, alkylsulfonyl, phenoxy, trialkylsilyloxy, or combinations thereof, heteroaryl which is unsubstituted or substituted one or more times by halogen, alkyl, alkenyl, alkynyl, hydroxy, alkoxy, alkoxyalkoxy, nitro, methylenedioxy, ethylenedioxy, trifluoromethyl, OCF₃, amino, aminoalkyl, aminoalkoxy, dialkylamino, hydroxyalkyl, hydroxamic acid, hydroxyalkoxy, carboxy, carboxyalkyl, alkoxycarbonyl, cyano, acyl, alkylthio, alkylsulfinyl, alkylsulfonyl, phenoxy, trialkylsilyloxy, or combinations thereof, or a heterocyclic group other than morpholinyl which is substituted or unsubstituted.

According to a further aspect of the invention, R¹⁴ in Formula II is cycloalkyl, aryl, heteroaryl, or a heterocyclic group.

According to a further aspect of the invention, R¹¹ in Formula II is OR¹⁶ and/or R¹² in Formula II is OR¹⁷.

According to a further aspect of the invention, E in Formula II is N or CH.

According to a further aspect of the invention, E in Formula II is N.

According to a further aspect of the invention, R¹¹ in Formula II is preferably halogen (e.g., F) or is preferably OR¹⁶, e.g., wherein R¹⁶ is alkyl (e.g., methyl), or halogenated alkyl (e.g., CF₃, CHF₂). In a more preferred embodiment, R¹¹ in Formula II is OR¹⁶, e.g., wherein R¹⁶ is alkyl (e.g., methyl), or halogenated alkyl (e.g., CHF₂).

According to a further aspect of the invention, R¹² in Formula II is preferably halogen (such as F or Cl) or is preferably OR¹⁷, e.g., wherein R¹⁷ is alkyl (such as methyl, ethyl, isopropyl), cycloalkyl (such as cyclobutyl, cyclopentyl, cyclohexyl), cycloalkylalkyl (such as cyclopropylmethyl), a heterocyclic group (such as tetrahydrofuranyl), or halogenated alkyl (e.g., CF₃, CHF₂). In a more preferred embodiment, R¹² in Formula II is OR¹⁷ e.g., wherein R¹⁷ is alkyl (such as methyl, ethyl, isopropyl), cycloalkyl (such as cyclopentyl, cyclohexyl), or cycloalkylalkyl (such as cyclopropylmethyl), especially alkyl or cycloalkylalkyl.

According to a further aspect of the invention, R¹³ in Formula II is preferably a fully saturated heterocyclic group having 5 to 10 ring atoms, preferably 5 to 8 ring atoms, in which at least 1 ring atom is an N, O or S atom, which is unsubstituted or substituted. In a more preferred embodiment R¹³ in Formula II is a fully saturated heterocyclic group having 5 to 10 ring atoms, particularly 5 to 8 ring atoms, which is substituted or unsubstituted, in which at least 1 ring atom is N (such as substituted or unsubstituted piperidinyl, (e.g., piperidin-4-yl, piperidin-3-yl) or substituted or unsubstituted pyrrolidinyl (e.g., pyrrolidin-2-yl, pyrrolidin-3-yl)).

According to a further aspect of the invention, R¹⁴ in Formula II is preferably cycloalkyl, aryl, heteroaryl or a heterocyclic group, which is substituted or unsubstituted, particularly cyclohexyl, piperidinyl, thienyl, or phenyl, especially phenyl, in each case substituted or unsubstituted. When R¹⁴ is phenyl, the preferred substituents are halogen (e.g., chloro, fluoro, bromo), alkyl (e.g., methyl), carboxy (e.g., 3-carboxy, 4-carboxy), alkoxy (e.g., methoxy), dialkylamino (e.g., dimethylamino), amido (e.g., CONH₂), cyano and/or M-R¹⁸, especially halogen, alkyl, carboxy, alkoxy, dialkylamino, CONH₂, and/or cyano, particularly halogen, alkyl, carboxy, alkoxy, dialkylamino, and/or cyano. Preferably, the phenyl is substituted at the 3- and/or 4-position.

According to a further aspect of the invention, R¹⁴ in Formula II is at least monosubstituted by R¹⁸-M- in which M is a single bond or a divalent aliphatic radical having 1 to 8 carbon atoms wherein at least one —CH₂— group is replaced by —SO₂NR¹⁹, —NR¹⁹—CO—, —CONR¹⁹—, —CO₂—, —CONHSO₂—, —SO₂NHCO—, —SO₂—, or —NR¹⁹SO₂— (e.g., the replacement may result in the divalent radical having no carbon atoms, i.e., where it is a single —CH₂— group which is replaced by, for example, —SO₂N¹⁹ or —NR¹⁹SO₂—). For example, M is a single bond or a divalent aliphatic radical having 1 to 8 carbon atoms wherein one —CH₂— group is replaced by —SO₂NR¹⁹, —NR¹⁹—, —CO₂—, —CONHSO₂—, —SO₂NHCO—, —SO₂—, or —NR¹⁹SO₂.

According to a further aspect of the invention, R¹⁸ in Formula II is preferably methyl, ethyl, propyl or phenyl, which in each case is unsubstituted or substituted.

According to a further aspect of the invention, R¹⁹ in Formula II is H, alkyl having 1 to 4 carbon atoms, or aryl.

According to a further aspect of the invention, R¹⁵ in Formula II is preferably H, F or methyl, more preferably H.

According to a further aspect of the invention, R¹¹ in Formula II is COR¹⁶, CONHR¹⁶ or NR¹⁶COR²⁰.

According to a further aspect of the invention, R¹² in Formula II is COR⁶, CONHR⁶ or NR¹⁶COR²⁰.

According to a further aspect of the invention, E is N or CH; R¹¹ is OR¹⁶; R¹² is OR¹⁷; R¹³ is a fully saturated heterocyclic group having 5 to 10 ring atoms, particularly 5-8 ring atoms, which is substituted or unsubstituted, in which at least 1 ring atom is N, such as substituted or unsubstituted piperidinyl, (e.g., piperidin-4-yl, piperidin-3-yl) or substituted or unsubstituted pyrrolidinyl (e.g., pyrrolidin-2-yl, pyrrolidin-3-yl); R⁴ is aryl or heteroaryl, each of which is substituted or unsubstituted (e.g., phenyl, chlorophenyl, methoxyphenyl, benzamide, carboxyphenyl, methylphenyl, dimethylaminophenyl, or thienyl); R¹⁶ is alkyl (e.g., methyl), or halogenated alkyl (e.g., CF₃, CHF₂); R¹⁷ is alkyl (such as methyl, ethyl, isopropyl), cycloalkyl (such as cyclopentyl, cyclohexyl), or cycloalkylalkyl (such as cyclopropylmethyl), and R¹⁴ is other than unsubstituted phenyl when R¹³ is substituted or unsubstituted piperidinyl.

According to a further aspect of the invention, E is N and R¹⁴ is other than unsubstituted phenyl when R¹³ is substituted or unsubstituted piperidinyl.

According to a further aspect of the invention, E is N, and when R¹³ is substituted or unsubstituted piperidinyl, then R¹⁴ is phenyl substituted by halogen, alkyl, carboxy, alkoxy, alkylamino, dialkylamino, nitro and/or cyano.

According to a further aspect of the invention, E is N or CR¹⁵ (e.g., CH), and R¹⁴ is other than unsubstituted phenyl when R¹³ is substituted or unsubstituted piperidinyl, wherein when R¹¹ is OR¹⁶ and R¹⁶ is alkyl, then R¹² is other than halogen, when R¹¹ is halogen, then R¹² is other than alkyl or fluorinated alkyl, and when R¹¹ is alkyl, then R¹² is other than alkyl.

According to a further aspect of the invention, E is N or CR¹⁵ (e.g., CH), and R¹⁴ is phenyl substituted by halogen, alkyl, carboxy, alkoxy, alkylamino, dialkylamino, nitro and/or cyano when R¹³ is substituted or unsubstituted piperidinyl, wherein when R¹¹ is OR¹⁶ and R¹⁶ is alkyl, then R¹² is other than halogen, when R¹¹ is halogen, then R¹² is other than alkyl or fluorinated alkyl, and when R¹¹ is alkyl, then R¹² is other than alkyl.

According to a further aspect of the invention, E is N or CR¹⁵ (e.g., CH), and R⁴ is other than unsubstituted phenyl when R¹³ is substituted or unsubstituted piperidinyl, wherein when R¹² is halogen, then R¹¹ is other than OR¹⁶, when R¹² is alkyl or fluorinated alkyl, then R¹¹ is other than halogen, and when R¹² is alkyl, then R¹¹ is other than alkyl.

According to a further aspect of the invention, E is N or CR¹⁵ (e.g., CH), and R¹⁴ is phenyl substituted by halogen, alkyl, carboxy, alkoxy, alkylamino, dialkylamino, nitro and/or cyano when R¹³ is substituted or unsubstituted piperidinyl, wherein when R¹² is halogen, then R¹¹ is other than OR¹⁶, when R¹² is alkyl or fluorinated alkyl, then R¹¹ is other than halogen, and when R¹² is alkyl, then R¹¹ is other than alkyl.

According to a further aspect of the invention, E is N or CR¹⁵ (e.g., CH); R¹¹ is alkyl, halogenated alkyl, OR¹⁶, COR¹⁶, CONHR¹⁶R²⁰, or NR¹⁶COR²⁰ (e.g., alkyl, halogenated alkyl, or OR₁₆; R¹² is alkyl, halogenated alkyl, OR¹⁷, COR¹⁶, CONHR¹⁶R²⁰, or NR⁶COR² (e.g., alkyl, halogenated alkyl, or OR¹⁷); R¹³ is a fully saturated heterocyclic group having 5 to 10 ring atoms, particularly 5-8 ring atoms, which is substituted or unsubstituted, in which at least 1 ring atom is N, such as substituted or unsubstituted piperidinyl, (e.g., piperinin-4-yl, piperidin-3-yl) or substituted or unsubstituted pyrrolidinyl (e.g., pyrrolidin-2-yl, pyrrolidin-3-yl); R¹⁴ is aryl or heteroaryl, each of which is substituted or unsubstituted (e.g., phenyl, chlorophenyl, methoxyphenyl, benzamide, carboxyphenyl, methylphenyl, dimethylaminophenyl, or thienyl); R¹⁶ is alkyl (e.g., methyl), or halogenated alkyl (e.g., CF₃, CHF₂); R¹⁷ is alkyl (such as methyl, ethyl, isopropyl), cycloalkyl (such as cyclopentyl, cyclohexyl), or cycloalkylalkyl (such as cyclopropylmethyl); and R¹¹ and R¹² are not both alkyl and R¹⁴ is other than unsubstituted phenyl when R¹³ is substituted or unsubstituted piperidinyl.

According to a further aspect of the invention, E is N or CR¹⁵ (e.g., CH); R¹¹ is alkyl, halogenated alkyl, OR¹⁶, COR¹⁶, CONHR¹⁶R¹⁰, or NR¹⁶COR²⁰ (e.g., alkyl, halogenated alkyl, or OR¹⁶); R¹² is alkyl, halogenated alkyl, OR¹⁷, COR¹⁶, CONHR¹⁶R²⁰, or NR¹⁶COR²⁰ (e.g., alkyl, halogenated alkyl, or OR¹⁷); R¹³ is a fully saturated heterocyclic group having 5 to 10 ring atoms, particularly 5-8 ring atoms, which is substituted or unsubstituted, in which at least 1 ring atom is N, such as substituted or unsubstituted piperidinyl, (e.g., piperidin-4-yl, piperidin-3-yl) or substituted or unsubstituted pyrrolidinyl (e.g., pyrrolidin-2-yl, pyrrolidin-3-yl); R¹⁴ is aryl or heteroaryl, each of which is substituted or unsubstituted (e.g., phenyl, chlorophenyl, methoxyphenyl, benzamide, carboxyphenyl, methylphenyl, dimethylaminophenyl, or thienyl); R¹⁶ is alkyl (e.g., methyl), or halogenated alkyl (e.g., CF₃, CHF₂); R¹⁷ is alkyl (such as methyl, ethyl, isopropyl), cycloalkyl (such as cyclopentyl, cyclohexyl), or cycloalkylalkyl (such as cyclopropylmethyl); and R¹¹ and R¹² are not both alkyl and R¹⁴ is phenyl substituted by halogen, alkyl, carboxy, alkoxy, alkylamino, dialkylamino, nitro and/or cyano when R¹³ is substituted or unsubstituted piperidinyl.

The present invention includes compounds of Formula III:

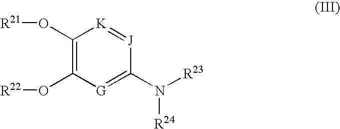

wherein
G, J, and K are each, independently, N or CR²⁵;
R²¹ is alkyl having 1 to 4 carbon atoms, which is branched or unbranched and which is unsubstituted or substituted one or more times by halogen (e.g., methyl, ethyl, isopropyl, CHF₂, CF₃, etc.);
R²² is alkyl having 1 to 12, preferably 1 to 8 carbon atoms, which is branched or unbranched and which is unsubstituted or substituted one or more times by halogen, hydroxy, cyano, C₁₋₄-alkoxy, oxo or combinations thereof, and wherein optionally one or more —CH₂CH₂— groups is replaced in each case by —CH═CH— or —C≡C— (e.g., CH₃, CHF₂, CF₃, methoxyethyl, etc.),
   cycloalkyl having 3 to 10, preferably 3 to 8 carbon atoms, which is unsubstituted or substituted one or more times by halogen, hydroxy, oxo, cyano, alkyl having 1 to 4 carbon atoms, alkoxy having 1 to 4 carbon atoms, or combinations thereof (e.g., cyclopentyl),
   cycloalkylalkyl having 4 to 16, preferably 4 to 12 carbon atoms, which is unsubstituted or substituted in the cycloalkyl portion and/or the alkyl portion one or more times by halogen, oxo, cyano, hydroxy, C₁₋₄-alkyl, C₁₋₄-alkoxy or combinations thereof (e.g., cyclopentylmethyl, cyclopropylmethyl, etc.),
   aryl having 6 to 14 carbon atoms, which is unsubstituted or substituted one or more times by halogen, CF₃, OCF₃, alkyl, hydroxy, alkoxy, nitro, methylenedioxy, ethylenedioxy, cyano, or combinations thereof (e.g., methylphenyl, methoxyphenyl, chlorophenyl, etc.),
   arylalkyl in which the aryl portion has 6 to 14 carbon atoms and the alkyl portion, which is branched or unbranched, has 1 to 5 carbon atoms, wherein the arylalkyl radical is unsubstituted or is substituted in the aryl portion one or more times by halogen, CF₃, OCF₃, alkyl, hydroxy, alkoxy, nitro, cyano, methylenedioxy, ethylenedioxy, or combinations thereof, and wherein in the alkyl portion one or more —CH₂CH₂— groups are each optionally replaced by —CH═CH— or —C≡C—, and one or more —CH₂— groups are each optionally replaced by —O— or —NH— and/or the alkyl portion is optionally substituted by halogen, oxo, hydroxy, cyano, or combinations thereof (e.g., phenylethyl, phenylpropyl, phenylbutyl, methoxyphenylethyl, methoxyphenylpropyl, chlorophenylethyl, chlorophenylpropyl, phenylethenyl, phenoxyethyl, phenoxybutyl, chlorophenoxyethyl, chlorophenylaminoethyl, etc.),
   a partially unsaturated carbocyclic group having 5 to 14 carbon atoms, which is unsubstituted or substituted one or more times by halogen, alkyl, alkoxy, hydroxy, nitro, cyano, oxo, or combinations thereof (e.g., cyclohexenyl, cyclohexadienyl, indanyl, tetrahydronaphthenyl, etc.),
   a heterocyclic group, which is saturated, partially saturated or unsaturated (e.g., heteroaryl), having 5 to 10 ring atoms in which at least 1 ring atom is a N, O or S atom, which is unsubstituted or substituted one or more times by halogen, hydroxy, aryl, alkyl, alkoxy, cyano, trifluoromethyl, nitro, oxo, or combinations thereof (e.g., 3-thienyl, 3-tetrahydrofuranyl, 3-pyrrolyl, etc.), or
   a heterocycle-alkyl group, wherein the heterocyclic portion is saturated, partially saturated or unsaturated (e.g., heteroaryl), and has 5 to 10 ring atoms in which at least 1 ring atom is a N, O or S atom, and the alkyl portion is branched or unbranched and has 1 to 5 carbon atoms, wherein the heterocycle-alkyl group is unsubstituted, substituted one or more times in the heterocyclic portion by halogen, OCF₃, hydroxy, aryl, alkyl, alkoxy, cyano, trifluoromethyl, nitro, oxo, or combinations thereof, and/or substituted one or more times in the alkyl portion by halogen, oxo, hydroxy, cyano, or combinations thereof, wherein in the alkyl portion one or more —CH₂CH₂— groups are each optionally replaced by —CH═CH— or —C≡C—, and one or more —CH₂— groups are each optionally replaced by —O— or —NH— (e.g., pyridylethyl, pydridylpropyl, methylpiperazinylethyl, etc.);
R²³ a non-aromatic heterocyclic group, which is fully saturated or partially saturated, having 5 to 10 ring atoms in which at least 1 ring atom is an N, O or S atom, which is unsubstituted or substituted one or more times by halogen, alkyl, hydroxy, alkoxy, alkoxyalkoxy, hydroxyalkyl-alkoxy, dihydroxyalkyl-alkoxy, nitro, oxo, methylenedioxy, ethylenedioxy, trifluoromethyl, OCF₃, amino, aminomethyl, aminoalkyl, aminoalkoxy, dialkylamino, hydroxyalkyl (e.g., hydroxymethyl), dihydroxyalkyl, hydroxamic acid (—C(O)—NHOH), tetrazole-5-yl, hydroxyalkoxy, carboxy, alkoxycarbonyl (e.g., tert-butyloxycarbonyl, ethoxycarbonyl), cyano, acyl (e.g., optionally substituted acetyl or optionally substituted benzoyl), alkylthio, alkylsulfinyl, alkylsulfonyl, phenylsulfonyl, phenoxy, cycloalkyl, aryl, heteroaryl or combinations thereof (e.g., piperidinyl, pyrrolydinyl, morpholinyl, piperazinyl, etc.),
R²⁴ is cycloalkyl having 3 to 10, preferably 3 to 8 carbon atoms, which is unsubstituted or substituted one or more times by halogen, hydroxy, oxo, cyano, alkyl having 1 to 4 carbon atoms, alkoxy having 1 to 4 carbon atoms, or combinations thereof (e.g., cyclopentyl),
   aryl having 6 to 14 carbon atoms and which is unsubstituted or substituted one or more times by halogen, alkyl, alkenyl, alkynyl, hydroxy, alkoxy, alkoxyalkoxy, nitro, methylenedioxy, ethylenedioxy, trifluoromethyl, OCF₃, amino, aminoalkyl, aminoalkoxy, dialkylamino, amido (e.g., CONH₂), hydroxyalkyl (e.g., hydroxymethyl), hydroxamic acid (—C(O)—NHOH), pyrrolyl, tetrazole-5-yl, 2(-heterocycle)tetrazole-5-yl (e.g., 2-(2-tetrahydropyranyl)tetrazole-5-yl), hydroxyalkoxy, carboxy, carboxyalkyl, alkoxycarbonyl (e.g., tert-butyloxycarbonyl, ethoxycarbonyl), cyano, acyl, alkylthio, alkylsulfinyl, alkylsulfonyl, phenoxy, trialkylsilyloxy (e.g. tert-butyldimethylsilyloxy), R²⁶-Q-, or combinations thereof (e.g., substituted or unsubstituted phenyl, naphthyl, and biphenyl, such as, but not limited to, phenyl, methylphenyl, chlorophenyl, fluorophenyl, methoxyphenyl, vinylphenyl, cyanophenyl, methylenedioxophenyl, ethylphenyl, dichlorophenyl, carboxyphenyl, ethoxycarbonylphenyl, dimethylphenyl, hydroxymethylphenyl, nitrophenyl, aminophenyl, dimethylaminophenyl, amidophenyl, etc.),
   heteroaryl having 5 to 10 ring atoms in which at least 1 ring atom is a heteroatom (e.g., N, S or O), which is unsubstituted or substituted one or more times by halogen, alkyl, hydroxy, alkoxy, alkoxyalkoxy, nitro, methylenedioxy, ethylenedioxy, trifluoromethyl, amino, aminomethyl, aminoalkyl, aminoalkoxy, dialkylamino, hydroxyalkyl (e.g., hydroxymethyl), hydroxamic acid (—C(O)—NHOH), tetrazole-5-yl, hydroxyalkoxy, carboxy, carboxyalkyl, alkoxycarbonyl (e.g., tert-butyloxycarbonyl, ethoxycarbonyl), cyano, acyl, alkylthio, alkylsulfinyl, alkylsulfonyl, phenoxy, trialkylsilyloxy (e.g. tert-butyldimethylsilyloxy), R²⁶-Q-, or combinations thereof (e.g., pyridyl, thienyl, pyrazinyl, quinolinyl, isoquinolinyl, pyrimidinyl, imidazolyl, thiazolyl, etc.),
   a heterocyclic group, which is saturated or partially saturated, having 5 to 10 ring atoms in which at least 1 ring atom is an N, O or S atom, which is unsubstituted or substituted one or more times by halogen, alkyl, hydroxy, alkoxy, alkoxyalkoxy, nitro, oxo, methylenedioxy, ethylenedioxy, trifluoromethyl, OCF₃, amino, aminomethyl, aminoalkyl, aminoalkoxy, dialkylamino, hydroxyalkyl (e.g., hydroxymethyl), hydroxamic acid (—C(O)—NOH), tetrazole-5-yl, hydroxyalkoxy, carboxy, alkoxycarbonyl (e.g., tert-butyloxycarbonyl, ethoxycarbonyl), cyano, acyl e.g., optionally substituted acetyl or optionally substituted benzoyl), alkylthio, alkylsulfinyl, alkylsulfonyl, phenylsulfonyl, phenoxy, cycloalkyl, aryl, heteroaryl or combinations thereof (e.g., piperidinyl, pyrrolydinyl, amidazolidinyl, pyrrolinyl, etc.), and
   a heterocycle-alkyl group, wherein the heterocyclic portion is saturated, partially saturated or unsaturated (e.g., heteroaryl), and has 5 to 10 ring atoms in which at least 1 ring atom is an N, O or S atom, and the alkyl portion is branched or unbranched and has 1 to 5 carbon atoms, wherein the heterocycle-alkyl group is unsubstituted, substituted one or more times in the heterocyclic portion by halogen, alkyl, hydroxy, alkoxy, alkoxyalkoxy, nitro, oxo, methylenedioxy, ethylenedioxy, trifluoromethyl, OCF₃, amino, aminomethyl, aminoalkyl, aminoalkoxy, dialkylamino, hydroxyalkyl (e.g., hydroxymethyl), hydroxamic acid (—C(O)—NHOH), tetrazole-5-yl, hydroxyalkoxy, carboxy, alkoxycarbonyl (e.g., tert-butyloxycarbonyl, ethoxycarbonyl), cyano, acyl alkylthio, alkylsulfinyl, alkylsulfonyl, phenylsulfonyl, phenoxy, cycloalkyl, aryl, heteroaryl or combinations thereof, and/or substituted in the alkyl portion one or more times by halogen, oxo, hydroxy, cyano, or combinations thereof, and wherein in the alkyl portion one or more —CH₂CH₂— groups are each optionally replaced by —CH═CH— or —C≡C—, and one or more —CH₂— groups are each optionally replaced by —O— or —NH— (e.g., pyridylethyl, pydridylpropyl, methylpiperazinylethyl, piperidinylmethyl, pyrrolydinylmethyl, amidazolidinylmethyl, pyrrolinylmethyl, etc.);
R²⁵ is H, halogen, alkyl having 1 to 4 carbon atoms, halogenated alkyl having 1 to 4 carbon atoms, alkoxy having 1 to 4 carbon atoms, or halogenated alkoxy having 1 to 4 carbon atoms;
R²⁶ is H,
   alkyl having 1 to 8, preferably 1 to 4 carbon atoms, which is unsubstituted or substituted one or more times by halogen, C₁₋₄-alkyl, C₁₋₄-alkoxy, oxo, or combinations thereof (e.g., methyl, ethyl, propyl, etc.),
   alkylamino or dialkylamino wherein each alkyl portion has independently 1 to 8, preferably 1 to 4 carbon atoms (e.g., dimethylamino, etc.),
   a partially unsaturated carbocycle-alkyl group wherein the carbocyclic portion has 5 to 14 carbon atoms and the alkyl portion has 1 to 5 carbon atoms, and which is unsubstituted or substituted, preferably in the carbocyclic portion, one or more times by halogen, alkyl, alkoxy, nitro, cyano, oxo, or combinations thereof (e.g., cyclohexenylmethyl, etc.),
   cycloalkyl having 3 to 10, preferably 3 to 8 carbon atoms, which is unsubstituted or substituted one or more times by halogen, hydroxy, oxo, cyano, alkoxy, alkyl having 1 to 4 carbon atoms, or combinations thereof (e.g., cyclopentyl),
   cycloalkylalkyl having 4 to 16, preferably 4 to 12 carbon atoms, which is unsubstituted or substituted in the cycloalkyl portion and/or the alkyl portion one or more times by halogen, oxo, cyano, hydroxy, alkyl, alkoxy or combinations thereof (e.g., cyclopentylmethyl, cyclopropylmethyl, etc.),
   aryl having 6 to 14 carbon atoms which is unsubstituted or substituted one or more times by halogen, alkyl, hydroxy, alkoxy, alkoxyalkoxy, nitro, methylenedioxy, ethylenedioxy, trifluoromethyl, amino, aminomethyl, aminoalkyl, aminoalkoxy, dialkylamino, hydroxyalkyl (e.g., hydroxymethyl), hydroxamic acid (—C(O)—NHOH), tetrazole-5-yl, hydroxyalkoxy, carboxy, alkoxycarbonyl (e.g., tert-butyloxycarbonyl, ethoxycarbonyl), cyano, acyl, alkylthio, alkylsulfinyl, alkylsulfonyl, phenoxy, cycloalkyl, aryl (e.g., phenyl, naphthyl, biphenyl), heteroaryl or combinations thereof (e.g., substituted or unsubstituted phenyl and naphthyl, methylphenyl, chlorophenyl, fluorophenyl, vinylphenyl, cyanophenyl, methylenedioxophenyl, ethylphenyl, dichlorophenyl, carboxyphenyl, ethoxycarbonylphenyl, dimethylphenyl, hydroxymethylphenyl, nitrophenyl, aminophenyl, etc.),
   arylalkyl having 7 to 19 carbon atoms, wherein the aryl portion has 6 to 14 carbon atoms and the alkyl portion, which is branched or unbranched, has 1 to 5 carbon atoms, wherein the arylalkyl radical is unsubstituted or substituted, in the aryl portion, one or more times by halogen, trifluoromethyl, CF₃O, nitro, amino, alkyl, alkoxy, amino, alkylamino, dialkylamino, or combinations thereof, and/or substituted in the alkyl portion by halogen, cyano, alkyl having 1 to 4 carbon atoms (e.g., methyl), or combinations thereof, wherein in the alkyl portion one or more —CH₂CH₂— groups are each optionally replaced by —CH═CH— or —C≡C—, and/or one or more —CH₂— groups are each optionally replaced by —O— or —NH— (e.g., benzyl, phenethyl, phenpropyl, methylbenzyl, methoxybenzyl, trifluoromethyl, benzyl, methylenedioxobenzyl, etc.),
   a heterocyclic group, which is saturated, partially saturated or unsaturated (e.g., heteroaryl), having 5 to 10 ring atoms in which at least 1 ring atom is an N, O or S atom, which is unsubstituted or substituted one or more times by halogen, alkyl, hydroxy, alkoxy, alkoxyalkoxy, nitro, methylenedioxy, ethylenedioxy, trifluoromethyl, amino, aminomethyl, aminoalkyl, aminoalkoxy, dialkylamino, hydroxyalkyl (e.g., hydroxymethyl), hydroxamic acid (—C(O)—NHOH), tetrazole-5-yl, hydroxyalkoxy, carboxy, alkoxycarbonyl (e.g., tert-butyloxycarbonyl, ethoxycarbonyl), cyano, acyl, alkylthio, alkylsulfinyl, alkylsulfonyl, phenoxy, cycloalkyl, aryl, heteroaryl or combinations thereof (e.g., pyridyl, thienyl, pyrazinyl, quinolinyl, isoquinolinyl, pyrimidinyl, imidazolyl, thiazolyl, etc.), or
   a heterocycle-alkyl group, wherein the heterocyclic portion is saturated, partially saturated or unsaturated (e.g., heteroaryl), and has 5 to 10 ring atoms in which at least 1 ring atom is an N, O or S atom, and the alkyl portion, which is branched or unbranched, has 1 to 5 carbon atoms, the heterocycle-alkyl group is unsubstituted, substituted one or more times in the heterocyclic portion by halogen, alkyl, alkoxy, cyano, trifluoromethyl, CF₃O, nitro, oxo, amino, alkylamino, dialkylamino, or combinations thereof and/or substituted one or more times in the alkyl portion by halogen, cyano, alkyl having 1 to 4 carbon atoms (e.g., methyl), or combinations thereof (e.g., pyridylmethyl, pyridylpropyl, methylpyridylmethyl, etc.);
Q is a single bond or a divalent aliphatic radical having 1 to 8 carbon atoms wherein one or more —CH₂— groups are each optionally replaced by —O—, —S—, —SO—, —SO₂—, —NR²⁷—, —SO₂NR²⁷—, —NR²⁷SO₂—, —CO—, —CO₂—, —NR²⁷CO—, —CONR²⁷—, —NHCONH—, —OCONH, —NHCOO—, —SCONH—, —SCSNH—, —NHCSNH—, —CONHSO₂— or —SO₂NHCO— (e.g., —O—, —CH₂—, —CO—, —CO—O—, —O—CO—, —CO—NH—, —NH—CO—, —CH₂CH₂CH₂—NH—CO—, —CH₂—CH₂—O—, —SO₂—NH—CH₂CH₂—O—, —O—CH₂CH₂—O—, —CH₂—NH—CO—, —CO—NH—CH₂—, —SO₂—NH—, —CH₂—NH—SO₂—, —CH₂CH₂CH₂—SO₂—NH—, —SO₂—, —CONHSO₂—, —SO₂NHCO—, etc.); and
R²⁷ is H,
   alkyl having 1 to 8, preferably 1 to 4 carbon atoms, which is branched or unbranched and which is unsubstituted or substituted one or more times with halogen, C₁₋₄-alkyl, C₁₋₄-alkoxy, oxo, or combinations thereof (e.g., methyl, ethyl, propyl, etc.),
   arylalkyl having 7 to 19 carbon atoms, wherein the aryl portion has 6 to 14 carbon atoms and the alkyl portion, which is branched or unbranched, has 1 to 5 carbon atoms, wherein the arylalkyl radical is unsubstituted or substituted, in the aryl portion, one or more times by halogen, trifluoromethyl, CF₃O, nitro, amino, alkyl, alkoxy, amino, alkylamino, dialkylamino, or combinations thereof, and/or substituted in the alkyl portion by halogen, cyano, alkyl having 1 to 4 carbon atoms (e.g., methyl), or combinations thereof, wherein in the alkyl portion one or more —CH₂CH₂— groups are each optionally replaced by —CH_CH— or —C≡C—, and/or one or more —CH₂— groups are each optionally replaced by —O— or —NH— (e.g., benzyl, phenethyl, phenpropyl, methylbenzyl, methoxybenzyl, trifluoromethyl, benzyl, methylenedioxobenzyl, etc.), or
   aryl having 6 to 14 carbon atoms and which is unsubstituted or substituted one or more times by halogen, alkyl, hydroxy, alkoxy, alkoxyalkoxy, nitro, methylenedioxy, ethylenedioxy, trifluoromethyl, amino, aminomethyl, aminoalkyl, aminoalkoxy dialkylamino, hydroxyalkyl (e.g., hydroxymethyl), hydroxamic acid (—C(O)—NHOH), tetrazole-5-yl, hydroxyalkoxy, carboxy, alkoxycarbonyl (e.g., tert-butyloxycarbonyl, ethoxycarbonyl), cyano, acyl, alkylthio, alkylsulfinyl, alkylsulfonyl, or combinations thereof (e.g., substituted or unsubstituted phenyl and naphthyl, methylphenyl, chlorophenyl, fluorophenyl, vinylphenyl, cyanophenyl, methylenedioxophenyl, ethylphenyl, dichlorophenyl, carboxyphenyl, ethoxycarbonylphenyl, dimethylphenyl, hydroxymethylphenyl, nitrophenyl, aminophenyl, etc.);

and pharmaceutically acceptable salts or solvates (e.g., hydrates) thereof or solvates of pharmaceutically acceptable salts thereof;

wherein if the compound exhibits chirality it can be in the form of a mixture of enantiomers such as a racemate or a mixture of diastereomers, or can be in the form of a single enantiomer or a single diastereomer;
wherein when R¹⁴ is pyridinylmethyl, R¹³ is other than unsubstituted or substituted piperidinyl, and

with the proviso that said compound is not:
6-[(3,4-dimethoxyphenyl)(2-furanylmethyl)amino]-1,2,4-triazine-3, 5 (2H,4H)dione,
N-(6-(cyclopentyloxy)-5-methoxy-2-pyridinyl]-N-4-piperidinyl-5-pyrimidinemethanamine,
6-(cyclopentyloxy)-5-methoxy-N-phenyl-N-piperidin-4-ylpyridin-2-amine,

or a pharmaceutically acceptable salt thereof, or solvate thereof, or solvate of a pharmaceutically acceptable salt thereof.

According to a further aspect of the invention, R²² is alkyl which is unsubstituted or substituted, cycloalkylalkyl which is unsubstituted or substituted, aryl which is unsubstituted or substituted, arylalkyl which is unsubstituted or substituted, a partially unsaturated carbocyclic group which is unsubstituted or substituted, a heterocyclic group which is unsubstituted or substituted, or a heterocycle-alkyl group which is unsubstituted or substituted; and R²⁴ is cycloalkyl which is unsubstituted or substituted, aryl which is unsubstituted or substituted one or more times by halogen, alkyl, alkenyl, alkynyl, hydroxy, alkoxy, alkoxyalkoxy, nitro, methylenedioxy, ethylenedioxy, trifluoromethyl, OCF₃, amino, aminoalkyl, aminoalkoxy, dialkylamino, hydroxyalkyl, hydroxamic acid, hydroxyalkoxy, carboxy, carboxyalkyl, alkoxycarbonyl, cyano, acyl, alkylthio, alkylsulfinyl, alkylsulfonyl, phenoxy, trialkylsilyloxy, or combinations thereof, heteroaryl which is unsubstituted or substituted one or more times by halogen, alkyl, alkenyl, alkynyl, hydroxy, alkoxy, alkoxyalkoxy, nitro, methylenedioxy, ethylenedioxy, trifluoromethyl, OCF₃, amino, aminoalkyl, aminoalkoxy, dialkylamino, hydroxyalkyl, hydroxamic acid, hydroxyalkoxy, carboxy, carboxyalkyl, alkoxycarbonyl, cyano, acyl, alkylthio, alkylsulfinyl, alkylsulfonyl, phenoxy, trialkylsilyloxy, or combinations thereof, or a heterocyclic group other than morpholinyl which is substituted or unsubstituted.

According to a further aspect of the invention, R²⁴ in Formula III is cycloalkyl, aryl, heteroaryl, or a heterocyclic group.

According to a further aspect of the invention, one of G, J, and K in Formula III is N (e.g., G is N) and the others are CR²⁵ (e.g., CH).

According to a further aspect of the invention, each of G, J, and K in Formula III is CR²⁵ (e.g., CH).

According to a further aspect of the invention, R²¹ in Formula III is preferably alkyl (e.g., methyl), or halogenated alkyl (e.g., CF₃, CHF₂).

According to a further aspect of the invention, R²² in Formula III is preferably alkyl (such as methyl, ethyl, isopropyl), cycloalkyl (such as cyclobutyl, cyclopentyl, cyclohexyl), cycloalkylalkyl (such as cyclopropylmethyl), a heterocyclic group (such as tetrahydrofuranyl), or halogenated alkyl (e.g., CF₃, CHF₂). In a more preferred embodiment, R²² in Formula II is alkyl (such as methyl, ethyl, isopropyl), cycloalkyl (such as cyclopentyl, cyclohexyl), or cycloalkylalkyl (such as cyclopropylmethyl), especially alkyl or cycloalkylalkyl.

According to a further aspect of the invention, R²³ in Formula III is preferably a fully saturated heterocyclic group having 5 to 10 ring atoms, preferably 5-8 ring atoms, in which at least 1 ring atom is an N, O or S atom, which is unsubstituted or substituted. In a more preferred embodiment, R²³ in Formula II is a fully saturated heterocyclic group having 5 to 10 ring atoms, which is substituted or unsubstituted, in which at least 1 ring atom is an N atom (such as substituted or unsubstituted piperidinyl, (e.g., piperidin-4-yl, piperidin-3-yl) or substituted or unsubstituted pyrrolidinyl (e.g., pyrrolidin-2-yl, pyrrolidin-3-yl).

According to a further aspect of the invention, R²⁴ in Formula III is preferably cycloalkyl, aryl, heteroaryl or a heterocyclic group, which is substituted or unsubstituted, particularly cyclohexyl, piperidinyl, thienyl, or phenyl, especially phenyl, in each case substituted or unsubstituted. When R²⁴ is phenyl, the preferred substituents are halogen (e.g., chloro, fluoro, bromo), alkyl (e.g., methyl), carboxy (e.g., 3-carboxy, 4-carboxy), alkoxy (e.g., methoxy), dialkylamino (e.g., dimethylamino), amido (e.g., CONH₂), cyano and/or Q-R²⁶, especially halogen, alkyl; carboxy, alkoxy, dialkylamino, CONH₂, and/or cyano, particularly halogen, alkyl, carboxy, alkoxy, dialkylamino, and/or cyano. Preferably, the phenyl is substituted at the 3- and/or 4-position.

According to a further aspect of the invention, R²³ in Formula III is at least monosubstituted by R²⁶-Q- in which Q is a single bond or a divalent aliphatic radical having 1 to 8 carbon atoms wherein at least one —CH₂— group is replaced by —SO²⁷, NR²⁷, NR²⁷CO—, —CONR²⁷—, —CO₂—, —CONSO₂—, —SO₂NHCO—, —SO₂—, or —NR²SO₂— (e.g., the replacement may result in the divalent radical having no carbon atoms, i.e., where it is a single —CH₂— group which is replaced by for example, —SO₂NR²⁷ or —NR²⁷SO₂—). For example, Q is a single bond or a divalent aliphatic radical having 1 to 8 carbon atoms wherein one —CH₂— group is replaced by —SO₂NR¹⁹, —NR¹⁹—, —CO₂—, —CONHSO₂—, —SO₂NHCO—, —SO₂—, or —NR¹⁹SO₂.

According to a further aspect of the invention, R²⁶ in Formula III is preferably methyl, ethyl, propyl or phenyl, which in each case is unsubstituted or substituted.

According to a further aspect of the invention, R²⁷ in Formula III is H, alkyl having 1 to 4 carbon atoms, or aryl.

According to a further aspect of the invention, R⁵ in Formula III is preferably H, F or methyl, more preferably H.

According to a further aspect of the invention, R²⁴ is other than unsubstituted phenyl when R²³ is substituted or unsubstituted piperidinyl, when G is CH, then K is other than CR²⁵ in which R²⁵ is alkoxy having 1 to 4 carbon atoms, and when K is CH, then G is other than CR²⁵ in which R²⁵ is alkoxy having 1 to 4 carbon atoms.

According to a further aspect of the invention, R²⁴ is phenyl substituted by halogen, alkyl, carboxy, alkoxy, alkylamino, dialkylamino, CONH₂, nitro and/or cyano when R²³ is substituted or unsubstituted piperidinyl, when C is CH, then K is other than CR²⁵ in which R²⁵ is alkoxy having 1 to 4 carbon atoms, and when K is CH, then G is other than CR²⁵ in which R²⁵ is alkoxy having 1 to 4 carbon atoms.

According to a further aspect of the invention, R²⁴ is other than unsubstituted phenyl when R²³ is substituted or unsubstituted piperidinyl, and when G is CH, then K is N or CR²⁵ in which R²⁵ is H, halogen, alkyl having 1 to 4 carbon atoms, halogenated alkyl having 1 to 4 carbon atoms, or halogenated alkoxy having 1 to 4 carbon atoms, and when K is CH, then G is N or CR²⁵ in which R²⁵ is H, halogen, alkyl having 1 to 4 carbon atoms, halogenated alkyl having 1 to 4 carbon atoms, or halogenated alkoxy having 1 to 4 carbon atoms.

According to a further aspect of the invention, R²⁴ is phenyl substituted by halogen, alkyl, carboxy, alkoxy, alkylamino, dialkylamino, CONH₂, nitro and/or cyano when R²³ is substituted or unsubstituted piperidinyl, and when G is CH, then K is N or CR²⁵ in which R²⁵ is H, halogen, alkyl having 1 to 4 carbon atoms, halogenated alkyl having 1 to 4 carbon atoms, or halogenated alkoxy having 1 to 4 carbon atoms, and when K is CH, then G is N or CR²⁵ in which R²⁵ is H, halogen, alkyl having 1 to 4 carbon atoms, halogenated alkyl having 1 to 4 carbon atoms, or halogenated alkoxy having 1 to 4 carbon atoms.

According to a further aspect of the invention, R²⁴ is substituted aryl or substituted or unsubstituted heteroaryl.

According to a further aspect of the invention, G and K are each N or CR²⁵; R²⁵ is H, halogen, alkyl having 1 to 4 carbon atoms, or halogenated alkyl having 1 to 4 carbon atoms; and R²⁴ in Formula III is cycloalkyl, aryl, heteroaryl other than pyrimidinyl, or a heterocyclic group.

According to a further compound and/or method aspect of the invention, the compounds of Formulas II and/or III are selected from:
15) N-(3-Chlorophenyl)-6-isopropoxy-5-methoxy-N-piperidin-4-ylpyridin-2-amine hydrochloride,
16) N-(3-chlorophenyl)-5-(difluoromethoxy)-6-ethoxy-N-piperidin-4-ylpyridin-2-amine hydrochloride,
17) 5-(difluoromethoxy)-methoxy-N-(4-methoxyphenyl)-N-piperidin-4-ylpyridin-2-amine,
18) 4-[(6-ethoxy-5-methoxypyridin-2-yl)(piperidin-4-yl)amino]benzonitrile,
19) 4-[(6-ethoxy-5-methoxypyridin-2-yl)(piperidin-4-yl)amino]benzamide,
20) 4-[[6-(cyclohexyloxy)-5-methoxypyridin-2-yl](piperidin-4-yl)amino]benzoic acid,
21) 6-(cyclopropylmethoxy)-5-methoxy-N-phenyl-N-piperidin-3-ylpyridin-2-amine,
22) 6-ethoxy-5-methoxy-N-piperidin-4-yl-N-3-thienylpyridin-2-amine,
23) 6-ethoxy-5-methoxy-N-(4-methylphenyl)-N-pyrrolidin-3-ylpyridin-2-amine,
24) N-(6-isobutoxy-4-methoxypyridin-2-yl)-N′,N′-dimethyl-N-piperidin-4-ylbenzene-1,4-diamine, and
25) N-(3-chlorophenyl)-N-[4-(difluoromethoxy)-3-methoxyphenyl]piperidin-4-amine,

and pharmaceutically acceptable salts thereof,

wherein a compound listed above (in either a free base form or in the form of a pharmaceutically acceptable salt thereof) can also be in the form of a solvate (such as a hydrate),

wherein a compound listed above (in a free base form or solvate thereof or in the form of a pharmaceutically acceptable salt or solvate thereof) can also be in the form of a polymorph, and

wherein if the compound exhibits chirality it can be in the form of a mixture of enantiomers such as a racemate or a mixture of diastereomers, or can be in the form of a single enantiomer or a single diastereomer.

The following table presents structures for several compounds of Formula II and/or III in accordance with the present invention:

| | Name | Structure |
|---|---|---|
| 15 | N-(3-chlorophenyl)-6-isopropoxy-5- methoxy-N-piperidin-4-ylpyridin-2- amine hydrochloride | 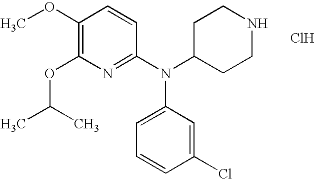 |
| 16 | N-(3-chlorophenyl)-5- (difluoromethoxy)-6-ethoxy-N- piperidin-4-ylpyridin-2-amine hydrochloride | 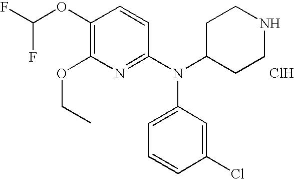 |
| 17 | 5-(difluoromethoxy)-6-methoxy-N-(4- methoxyphenyl)-N-piperidin-4- ylpyridin-2-amine | 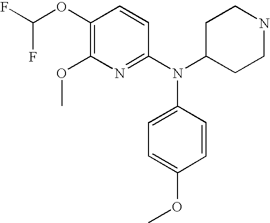 |
| 18 | 4-[(6-ethoxy-5-methoxypyridin-2- yl)(piperidin-4-yl)amino]benzonitrile | 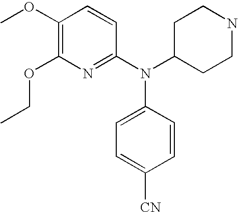 |
| 19 | 4-[(6-ethoxy-5-methoxypyridin-2- yl)(piperidin-4-yl)amino]benzamide | 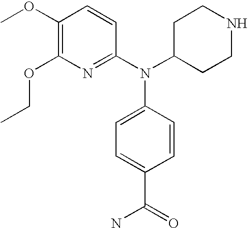 |
| 20 | 4-[[6-(cyclohexyloxy)-5- methoxypyridin-2-yl](piperidin-4- yl)amino]benzoic acid | 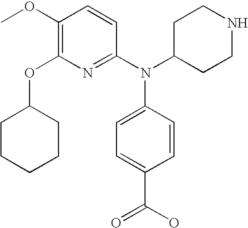 |
| 21 | 6-(cyclopropylmethoxy)-5-methoxy-N- phenyl-N-piperidin-3-ylpyridin-2- amine | 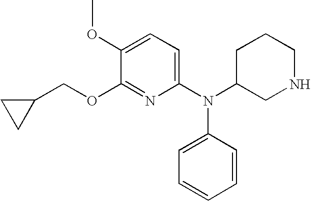 |
| 22 | 6-ethoxy-5-methoxy-N-piperidin-4-yl- N-3-thienylpyridin-2-amine | 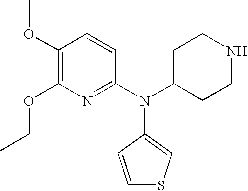 |
| 23 | 6-ethoxy-5-methoxy-N-(4- methylphenyl)-N-pyrrolidin-3- ylpyridin-2-amine | 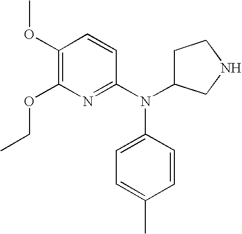 |
| 24 | N-(6-isobutoxy-5-methoxypyridin-2- yl)-N′,N′-dimethyl-N-piperidin-4- ylbenzene-1,4-diamine | 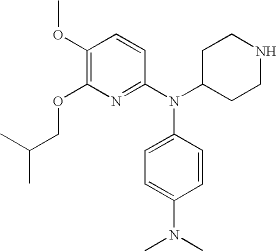 |
| 25 | N-(3-chlorophenyl)-N-[4- (difluoromethoxy)-3- methoxyphenyl]piperidin-4-amine | 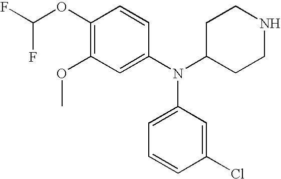 |

In a further compound and/or method aspect of the present invention, the compound is selected from:
26) 6-(cyclopentyloxy)-5-methoxy-N-phenyl-N-piperidin-4-ylpyridin-2-amine.

According to a further aspect of the invention there is provided a genus of novel compounds according to the Formulas IV, V, VI, VII, VIII, and IX:

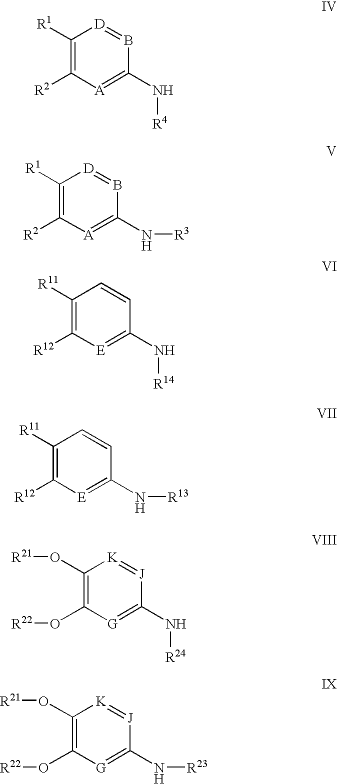

and pharmaceutically acceptable salts or solvates (e.g., hydrates) thereof; or solvates of pharmaceutically acceptable salts thereof; wherein A, B, D, R¹R², R³, and R⁴ are as defined above in Formula I, E, R¹¹, R¹², R¹³, and R¹⁴ are as defined above in Formula II, and G, J, K, R²¹, R²², R²³, and R²⁴ are as defined above in Formulas IV-IX not only have PDE4 inhibitory activity, but also are useful as intermediates for preparing compounds of Formula I in which R³ and R⁴ are both other than H, for preparing compounds of Formula II, in which R¹³ and R¹⁴ are both other than H, and for preparing compounds of Formula III in which R²³ and R²⁴ are both other than H.

In addition, other preferred compounds of Formula I are those of subformulas X, XI, XII, and XIII:

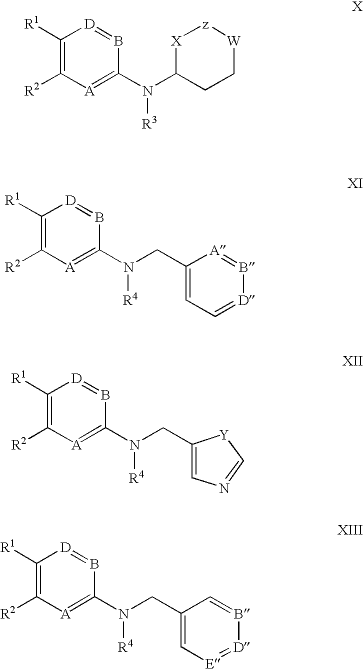

wherein, A, B, D, R¹, R², R³, and R⁴ in Formulas X-XIII are as defined in Formula I.

In Formula X, one or more of X, Z and W is S, O, NH, or N which is substituted (e.g., by alkyl or halogenated alkyl), and the others are each CH₂. Preferably, X and Z are CH₂ and W is NH. Also, R³ is preferably arylalkyl (e.g., benzyl) which is substituted or unsubstituted, or heteroarylalkyl, which is substituted or unsubstituted. Preferred arylalkyl substituents include halogen (e.g., fluoro, chloro, bromo), carboxy, cyano, tetrazole and/or L-R⁸.

In Formulas XI, XII, and XIII, one or two of A″, B″, D″, and E″ is N or N—O and the others are each CH, and Y is S, O, NH, or N which is substituted (e.g., by alkyl or halogenated alkyl), preferably S or O. Preferably, B″ is N or N—O. Also, 4 is preferably phenyl which is substituted or unsubstituted. Preferred phenyl substituents are carboxy, cyano, tetrazole and/or L-R⁸.

The compounds of the present invention are effective in inhibiting, or modulating the activity of PDE4 in patients, e.g., mammals, especially humans. These compounds exhibit neurological activity, especially where such activity affects cognition, including long term memory. These compounds will also be effective in treating diseases where decreased cAMP levels are involved. This includes, but is not limited to, inflammatory diseases. These compounds may also function as antidepressants, or be useful in treating cognitive and negative symptoms of schizophrenia.

Assays for determining PDE inhibiting activity as well as selectivity of PDE 4 inhibiting activity and selectivity of inhibiting PDE 4 isoenzymes are known within the art. See, e.g., U.S. Pat. No. 6,136,821, the disclosure of which is incorporated herein by reference.

According to a further aspect of the invention there are provided compounds useful as intermediates for the production of the PDE4 inhibitors described herein (e.g., PDE4 inhibitors of Formulas I-III) and/or useful for the synthesis of radio-labeled analogs of the PDE4 inhibitors within this application.

Thus, there are provided intermediate compounds which correspond to compounds of Formula I-III, wherein R², R³, and R⁴ are as previously defined for Formula I, R¹², R¹³, and R¹⁴ are as previously defined for Formula II, and R²², R²³, and R²⁴ are as previously defined for Formula III, and R¹ in Formula I is OR⁶ and R²¹ in Formula II is OR¹⁶, but R⁶ in Formula I, R¹⁶ in Formula II, and R²¹ in Formula III is H, tert-butyldimethylsilyl-, or a suitable phenolic protecting group. Suitable phenolic protecting groups are described, for example, in Greene, T. W. and Wuts, P. G. M., Protective Groups in Organic Synthesis, 3^{rd} Edition, John Wiley & Sons, 1999, pp. 246-293. These intermediates are also useful for the synthesis of radio-labeled compounds, such as where R⁶, R¹⁶ and/or R²¹ is ³H₃C—, ¹⁴CH₃— or ¹¹CH₃—, for example, by removing the protecting group and reacting the resultant compound in which R⁶ is H with suitable radio-labeled reagents. Such radio-labeled compounds are useful for determining compound tissue distribution in animals, in PET imaging studies, and for in vivo, ex vivo, and in vitro binding studies.

Also provided intermediate compounds which correspond to compounds of Formula I-III, wherein R¹, R³, and R⁴ are as previously defined for Formula I, R¹¹, R¹³, and R¹⁴ are as previously defined for Formula II, and R²¹, R²³, and R²⁴ are as previously defined for Formula III, and R² in Formula I is OR⁷ and R²² in Formula II is OR¹⁷, but R⁷ in Formula I, R¹⁷ in Formula II, and R²² in Formula III is H, tert-butyldimethylsilyl-, or a suitable phenolic protecting group. Suitable phenolic protecting groups are described, for example, in Greene, T. W. and Wuts, P. G. M., Protective Groups in Organic Synthesis, 3^{rd} Edition, John Wiley & Sons, 1999, pp. 246-293. Compounds in which R⁷, R¹⁷ and R²² is H are useful as intermediates, for example, as scaffolds for parallel or combinatorial chemistry applications. Further, these compounds are useful for the introduction of radio-labels such as ³H, ¹⁴C, or ¹¹C.

Halogen herein refers to F, Cl, Br, and I. Preferred halogens are F and Cl.

Alkyl, as a group or substituent per se or as part of a group or substituent (e.g., alkylamino, trialkylsilyloxy, aminoalkyl, hydroxyalkyl), means a straight-chain or branched-chain aliphatic hydrocarbon radical having 1 to 12 carbon atoms, preferably 1 to 8 carbon atoms, especially 1 to 4 carbon atoms. Suitable alkyl groups include, but are not limited to, methyl, ethyl, propyl, isopropyl, butyl, sec-butyl, tert-butyl, pentyl, hexyl, heptyl, octyl, nonyl, decyl, undecyl, and dodecyl. Other examples of suitable alkyl groups include, but are not limited to, 1-, 2- or 3-methylbutyl, 1,1-, 1,2- or 2,2-dimethylpropyl, 1-ethylpropyl, 1-, 2-, 3- or 4-methylpentyl, 1,1-, 1,2-, 1,3-, 2,2-, 2,3- or 3,3-dimethylbutyl, 1- or 2-ethylbutyl, ethylmethylpropyl, trinmethylpropyl, methylhexyl, dimethylpentyl, ethylpentyl, ethylmethylbutyl, dimethylbutyl, and the like.

Substituted alkyl groups are alkyl groups as described above which are substituted in one or more positions by halogens, oxo, hydroxyl, C₁₋₄-alkoxy and/or cyano. Halogens are preferred substituents, especially F and Cl.

Alkoxy means alkyl-O— groups and alkoxyalkoxy means alkyl-O-alkyl-O— groups in which the alkyl portions are in accordance with the previous discussion. Suitable alkoxy and alkoxyalkoxy groups include, but are not limited to, methoxy, ethoxy, propoxy, butoxy, pentoxy, hexoxy, heptoxy, octoxy, methoxymethoxy, ethoxymethoxy, propoxymethoxy, and methoxyethoxy. Preferred alkoxy groups are methoxy and ethoxy. Similarly, alkoxycarbonyl means alkyl —O—CO— in which the alkyl portion is in accordance with the previous discussion. Examples include, but are not limited to, methoxycarbonyl, ethoxycarbonyl, propoxycarbonyl, and tert-butoxycarbonyl.

Cycloalkyl means a monocyclic, bicyclic or tricyclic nonaromatic saturated hydrocarbon radical having 3 to 10 carbon atoms, preferably 3 to 8 carbon atoms, especially 3 to 6 carbon atoms. Suitable cycloalkyl groups include, but are not limited to, cyclopropyl, cyclobutyl, cyclopentyl, cyclohexyl, cycloheptyl, cyclooctyl, norbornyl, 1-decalin, adamant-1-yl, and adamant-2-yl. Other suitable cycloalkyl groups include, but are not limited to, spiropentyl, bicyclo[2.1.0]pentyl, bicyclo[3.1.0]hexyl, spiro[2.4]heptyl, spiro[2.5]octyl, bicyclo[5.1.0]octyl, spiro[2.6]nonyl, bicyclo[2.2.0]hexyl, spiro[3.3]heptyl, bicyclo[4.2.0]octyl, and spiro[3.5]nonyl. Preferred cycloalkyl groups include cyclopropyl, cyclobutyl, cyclopentyl and cyclohexyl. The cycloalkyl group can be substituted, for example, by one or more halogens and/or alkyl groups.

Cycloalkylalkyl refers to cycloalkyl-alkyl radicals in which the cycloalkyl and alkyl portions are in accordance with previous discussions. Suitable examples include, but are not limited to, cyclopropylmethyl and cyclopentylmethyl.

Aryl, as a group or substituent per se or as part of a group or substituent, refers to an aromatic carbocyclic radical containing 6 to 14 carbon atoms, preferably 6 to 12 carbon atoms, especially 6 to 10 carbon atoms. Suitable aryl groups include, but are not limited to, phenyl, naphthyl and biphenyl. Substituted aryl groups include the above-described aryl groups which are substituted one or more times by, for example, halogen, alkyl, hydroxy, alkoxy, nitro, methylenedioxy, ethylenedioxy, amino, alkylamino, dialkylamino, hydroxyalkyl, hydroxyalkoxy, carboxy, cyano, acyl, alkoxycarbonyl, alkylthio, alkylsulfinyl, alkylsulfonyl, phenoxy, and combinations thereof.

Arylalkyl refers to an aryl-alkyl-radical in which the aryl and alkyl portions are in accordance with the previous descriptions. Suitable examples include, but are not limited to, benzyl, 1-phenethyl, 2-phenethyl, phenpropyl, phenbutyl, phenpentyl, and napthylmethyl.

Substituted arylalkyl refers to an arylalkyl group, where the aryl portion and/or the alkyl portions are substituted in accordance with the definitions given above.

Heteroaryl refers to an aromatic heterocyclic group having one or two rings and a total number of 5 to 10 ring atoms wherein at least one of the ring atoms is a heteroatom. Preferably, the heteroaryl group contains 1 to 3, especially 1 or 2, hetero-ring atoms which are selected from N, O and S. Suitable heteroaryl groups include, but are not limited to, furyl, thienyl, pyrrolyl, pyrazolyl, imidazolyl, triazolyl, tetrazolyl, isoxazolyl, oxazolyl, thiazolyl, isothiazolyl, oxadiazolyl, oxatriazolyl, thiadiazolyl, pyridyl, pyridazinyl, pyrimidinyl, pyrazinyl, triazinyl, benzofuranyl, isobenzofuranyl, thionaphthenyl, isothionaphthenyl, indolyl, isoindolyl, indazolyl, benzisoxazolyl, benzoxazolyl, benzthiazolyl, benzisothiazolyl, purinyl, benzopyranyl, quinolinyl, isoquinolinyl, cinnolinyl, quinazolinyl, naphthyridinyl, and benzoxazinyl, e.g., 2-thienyl, 3-thienyl, 2-, 3- or 4-pyridyl, 2-, 3-, 4-, 5-, 6-, 7- or 8-quinolinyl, and 1-, 3-, 4-, 5-, 6-, 7- or 8-isoquinolinyl.

Substituted heteroaryl refers to the heteroaryl groups described above which are substituted in one or more places by, for example, halogen, aryl, alkyl, alkoxy, carboxy, methylene, cyano, trifluoromethyl, nitro, amino, alkylamino, and dialkylamino.

Heterocycles include heteroaryl groups as described above as well as non-aromatic cyclic groups containing at least one hetero-ring atom, preferably selected from N, S and O, for example, but not limited to, tetrahydrofuranyl, piperidinyl, dithialyl, oxathialyl, dioxazolyl, oxathiazolyl, oxazinyl, isoxazinyl, oxathiazinyl, oxadiazinyl, and pyrrolidinyl.

Heterocycle-alkyl refers to a heterocycle-alkyl-group wherein the heterocyclic and alkyl portions are in accordance with the previous discussions. Suitable examples include, but are not limited to, pyridylmethyl, thiazolylmethyl, thienylmethyl, pyrimidinylmethyl, pyrazinylmethyl, and isoquinolinylmethyl.

Partially unsaturated carbocyclic structures are non-aromatic monocyclic or bicyclic structures containing 5 to 14 carbon atoms, preferably 6 to 10 carbon atoms, wherein the ring structure(s) contains at least one C≡C bond. Suitable examples include, but are not limited to, cyclopentenyl, cyclohexenyl, cyclohexadienyl, tetrahydronaphthenyl and indan-2-yl.

Alkenyl refers to straight-chain or branched-chain aliphatic radicals containing 2 to 12 carbon atoms in which one or more —CH₂—CH₂— structures are each replaced by —CH═CH—. Suitable alkenyl groups include, but are not limited to, ethenyl, 1-propenyl, 2-methylethenyl, 1-butene, 2-butene, 1-pentenyl, and 2-pentenyl.

Alkynyl refers to straight-chain or branched-chain aliphatic radicals containing 2 to 12 carbon atoms in which one or more —CH₂—CH₂— structures are each replaced by —C≡C—. Suitable alkynyl groups include, but are not limited to, ethynyl, propynyl, 1-butynyl, and 2-butynyl.

Acyl refers to alkanoyl radicals having 1 to 13 carbon atoms in which the alkyl portion can be substituted by halogen, alkyl, aryl and/or alkoxy, or aroyl radicals having 7 to 15 carbon atoms in which the aryl portion can be substituted by, for example, halogen, alkyl and/or alkoxy. Suitable acyl groups include formyl, acetyl, propionyl, butanoyl and benzoyl.

Amido refers to —CONR′R″ radicals where R′ and R″ are each, independently, H or alkyl having 1 to 4 carbon atoms, preferably H.

Substituted radicals preferably have 1 to 3 substituents, especially 1 to 2 substituents.

Additional aspects of the present invention include pharmaceutical compositions comprising a compound of Formulas I-III and a pharmaceutically acceptable carrier and, optionally, another active agent as discussed below; a method of inhibiting a PDE4 enzyme, especially an isoenzyme, e.g., as determined by a conventional assay or one described herein, either in vitro or in vivo (in an animal, e.g., in an animal model, or in a mammal or in a human); a method of treating neurological syndrome, e.g., loss of memory, especially long-term memory, cognitive impairment or decline, memory impairment, etc. a method of treating a disease state modulated by PDE4 activity, in a mammal, e.g., a human, e.g., those mentioned herein.

The compounds of the present invention may be prepared conventionally. Some of the processes which can be used are described below. All starting materials are known or can be conventionally prepared from known starting materials.

The reaction schemes shown below are for illustrative purposes only and should not be viewed as limiting the scope of the synthetic methods available for the production of the compounds described within this application. Note that alternative methods, reagents, solvents, bases, acids etc., which are considered standard in the art, can be utilized in addition or can replace those mentioned here, to prepare many of the compounds described below.

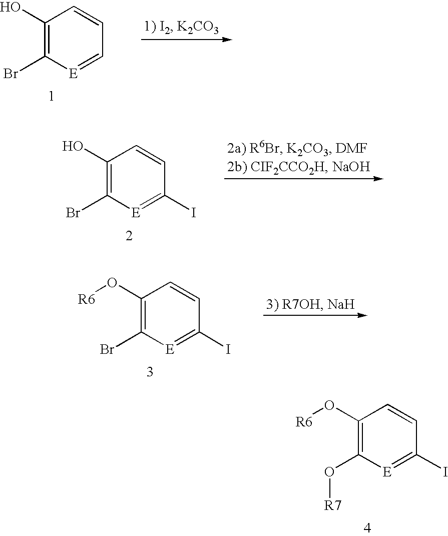

For compounds where E is N, starting material 2,3-diether-6-iodopyridines 4 may be prepared in a three step procedure from commercially available 2-bromo-3-hydroxypyridine 1. Thus, selective 6-iodoination (12, K₂CO₃) followed by etherification generates 2-bromo-6-iodopyridines 3 (Koch, V., Schnatter, S., Synthesis, 1990, 497-498). Reaction with a sodium alkoxide (R⁷ONa) provides 2,3-diether-6-iodopyridines 4 (O'Neill, B. T., Yohannes, D., Bundesmann, M. W., Arnold, E. P., Org. Lett., 2000, 2(26), 4201-4204).

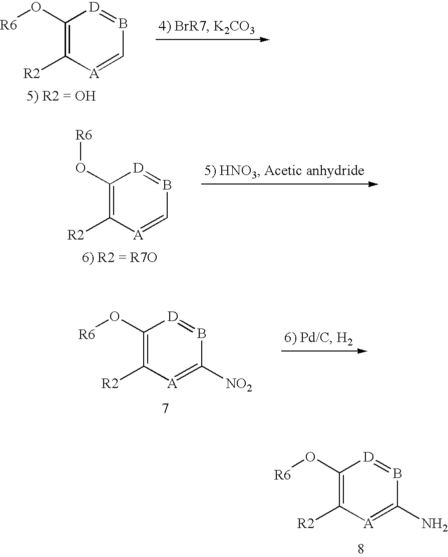

Starting anilines 8 may be prepared in a three-step procedure from various 2-alkyloxyphenols 5. Thus phenol S undergoes reaction with an alkylhalide in the presence of a base such as K₂CO₃ to yield substituted dietherbenzenes 6. Nitration reaction generates nitrocatechols 7, which are subsequently reduced by catalytic hydrogenation over Pd/C to provide corresponding anilines 8.

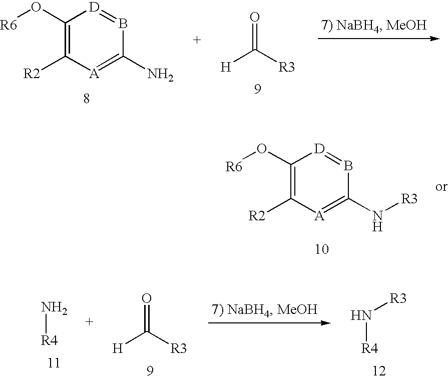

Reductive amination between aniline precursors 8 and aldehydes 9 provide key intermediates 10 in high yield. Alternatively, secondary amines 12 may be formed by reductive amination between amines 11 and aldehydes 9,

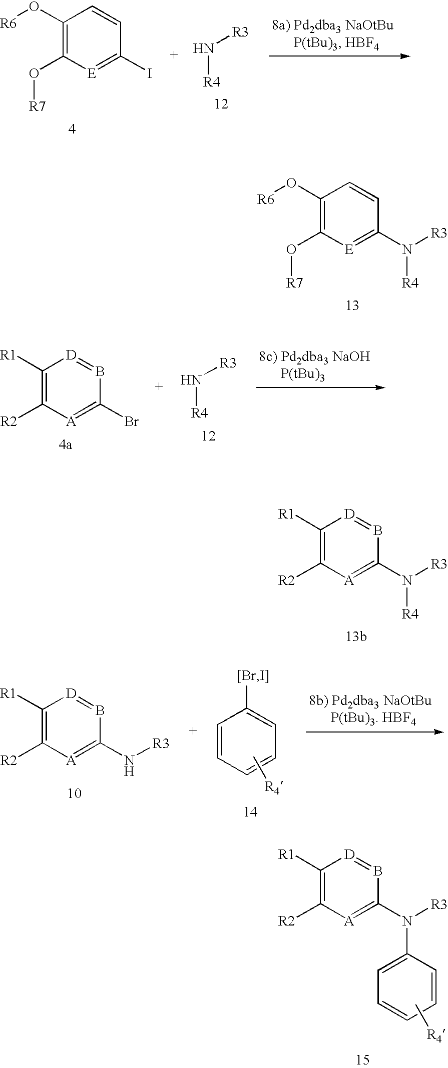

Buchwald N-arylation reaction between reductive amination product 12 and 6-iodopyridine 4, bromo compound 4a and amine 12, or reductive amination product 10 and an aryl- or heteroaryl-halide 14 provides key targets and intermediates of the general type 13, 13b and 15 respectively (Hartwig, J. F., Kawatsura, M., Hauck, S. I., Shaughnessy, K. H., Alcazar-Roman, L. M., J. Org. Chem., 1999, 64, 5575-5580). Compounds of the type 16 where R₄′ is CO₂tBu can be converted to the corresponding acid 17 by stirring in a solution of 20% TFA in DCM, or by base hydrolysis (NaOH, MeOH). When R₄′ is a THP-protected tetrazole 18, the THP group is removed by treating with 3N HCl to provide the tetrazole compounds of type 19 (Greene, T. W., P. G. M., Protective Groups in Organic Synthesis, Third Edition, John Wiley & Sons, Inc. New York, pp. 49-54 and 404-408).

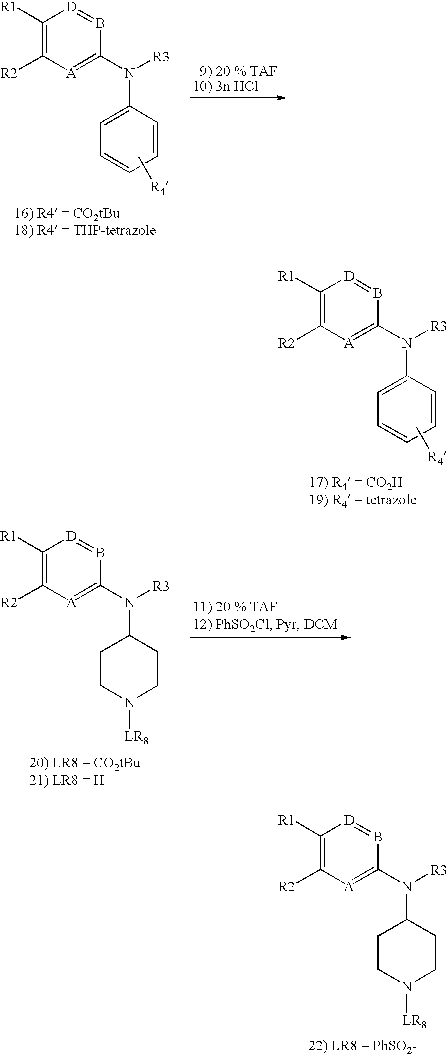

Boc-protected piperidines 20 are unmasked by treating with 20% TFA in DCM to generate piperidine analogs 21. These piperidines undergo reaction with various acid chlorides and sulfonyl chlorides to provide targets such as 22.

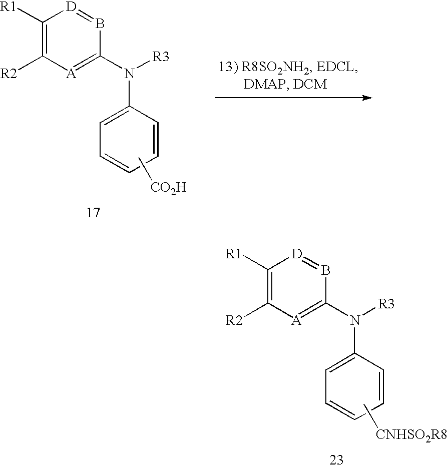

Alternatively, acids 17 undergo reaction with various amine compounds to generate sulfonylaminocarbonyl targets 23 by coupling reaction with a sulfonamide in the presence of EDCI and DMAP.

Many of these synthetic procedures are described more fully in the examples below.

One of ordinary skill in the art will recognize that some of the compounds of Formulas I-III and the specific compounds listed above can exist in different geometrical isomeric forms. In addition, some of the compounds of the present invention possess one or more asymmetric carbon atoms and are thus capable of existing in the form of optical isomers, as well as in the form of racemic or nonracemic mixtures thereof and in the form of diastereomers and diastereomeric mixtures inter alia. All of these compounds, including cis isomers, trans isomers, diastereomic mixtures, racemates, nonracemic mixtures of enantiomers, and substantially pure and pure enantiomers, are within the scope of the present invention. Substantially pure enantiomers contain no more than 5% w/w of the corresponding opposite enantiomer, preferably no more than 2%, most preferably no more than 1%.

The optical isomers can be obtained by resolution of the racemic mixtures according to conventional processes, for example, by the formation of diastercoisomeric salts using an optically active acid or base or formation of covalent diastereomers. Examples of appropriate acids include tartaric, diacetyltartaric, dibenzoyltartaric, ditoluoyltartaric and camphorsulfonic acid. Mixtures of diastereoisomers can be separated into their individual diastereomers on the basis of their physical and/or chemical differences by methods known to those skilled in the art, for example, by chromatography or fractional crystallization. The optically active bases or acids are then liberated from the separated diastereomeric salts. A different process for separation of optical isomers involves the use of chiral chromatography (e.g., chiral HPLC columns), with or without conventional derivation, optimally chosen to maximize the separation of the enantiomers. Suitable chiral HPLC columns are manufactured by Diacel, e.g., Chiracel OD and Chiracel OJ among many others, all routinely selectable. Enzymatic separations, with or without derivitization, are also useful. The optically active compounds of Formulas I-III can likewise be obtained by chiral syntheses utilizing optically active starting materials.

In addition, one of ordinary skill in the art will recognize that the compounds can be used in different enriched isotopic forms, e.g., enriched in the content of ²H, ³H, ¹¹C and/or ¹⁴C.

The present invention also relates to useful forms of the compounds as disclosed herein, such as pharmaceutically acceptable salts and prodrugs of all the compounds of the present invention. Pharmaceutically acceptable salts include those obtained by reacting the main compound, functioning as a base, with an inorganic or organic acid to form a salt, for example, salts of hydrochloric acid, sulfuric acid, phosphoric acid, methane sulfonic acid, camphor sulfonic acid, oxalic acid, maleic acid, succinic acid and citric acid. Pharmaceutically acceptable salts also include those in which the main compound functions as an acid and is reacted with an appropriate base to form, e.g., sodium, potassium, calcium, magnesium, ammonium, and choline salts. Those skilled in the art will further recognize that acid addition salts of the claimed compounds may be prepared by reaction of the compounds with the appropriate inorganic or organic acid via any of a number of known methods. Alternatively, alkali and alkaline earth metal salts are prepared by, for example, reacting a compound of the invention with the appropriate base via a variety of known methods.

The following are further non-limiting examples of acid salts that can be obtained by reaction with inorganic or organic acids: acetates, adipates, alginates, citrates, aspartates, benzoates, benzenesulfonates, bisulfates, butyrates, camphorates, digluconates, cyclopentanepropionates, dodecylsulfates, ethanesulfonates, glucoheptanoates, glycerophosphates, hemisulfates, heptanoates, hexanoates, fumarates, hydrobromides, hydroiodides, 2-hydroxy-ethanesulfonates, lactates, maleates, methanesulfonates, nicotinates, 2-naphthalenesulfonates, oxalates, palmoates, pectinates, persulfates, 3-phenylpropionates, picrates, pivalates, propionates, succinates, tartrates, thiocyanates, tosylates, mesylates and undecanoates.

Thus, examples of suitable salts include hydrochloride, oxalate, hydroformate and trifluoroacetate salts.

Preferably, the salts formed are pharmaceutically acceptable for administration to patients, such as mammals, e.g., humans. However, pharmaceutically unacceptable salts of the compounds are suitable as intermediates, for example, for isolating the compound as a salt and then converting the salt back to the free base compound by treatment with an alkaline reagent. The free base can then, if desired, be converted to a pharmaceutically acceptable acid addition salt.

One of ordinary skill in the art will also recognize that some of the compounds of Formulas I-III can exist in different polymorphic forms. As known in the art, polymorphism is an ability of a compound to crystallize as more than one distinct crystalline or “polymorphic” species. A polymorph is a solid crystalline phase of a compound with at least two different arrangements or polymorphic forms of that compound molecule in the solid state. Polymorphic forms of any given compound are defined by the same chemical formula or composition and are as distinct in chemical structure as crystalline structures of two different chemical compounds.

One of ordinary skill in the art will further recognize that compounds of Formulas I-III can exist in different solvate forms. Solvates of the compounds of the invention may also form when solvent molecules are incorporated into the crystalline lattice structure of the compound molecule during the crystallization process. For example, suitable solvates include hydrates, e.g., monohydrates, dihydrates, sesquihydrates, and hemihydrates.

The compounds of the invention can be administered alone or as an active ingredient of a formulation. Thus, the present invention also includes pharmaceutical compositions of compounds of Formula I-III containing, for example, one or more pharmaceutically acceptable carriers.

Numerous standard references are available that describe procedures for preparing various formulations suitable for administering the compounds according to the invention. Examples of potential formulations and preparations are contained, for example, in the Handbook of Pharmaceutical Excipients, American Pharmaceutical Association (current edition); Pharmaceutical Dosage Forms: Tablets (Lieberman, Lachman and Schwartz, editors) current edition, published by Marcel Dekker, Inc., as well as Remington's Pharmaceutical Sciences (Arthur Osol, editor), 1553-1593 (current edition).

In view of their high degree of PDE4 inhibition, the compounds of the present invention can be administered to any patient requiring or desiring PDE4 inhibition, and/or enhancement of cognition. Administration may be accomplished according to patient needs, for example, orally, nasally, parenterally (subcutaneously, intravenously, intramuscularly, intrasternally and by infusion), by inhalation, rectally, vaginally, topically, locally, transdermally, and by ocular administration.

Various solid oral dosage forms can be used for administering compounds of the invention including such solid forms as tablets, gelcaps, capsules, caplets, granules, lozenges and bulk powders. The compounds of the present invention can be administered alone or combined with various pharmaceutically acceptable carriers, diluents (such as sucrose, mannitol, lactose, starches) and excipients known in the art, including but not limited to suspending agents, solubilizers, buffering agents, binders, disintegrants, preservatives, colorants, flavorants, lubricants and the like. Time release capsules, tablets and gels are also advantageous in administering the compounds of the present invention.

Various liquid oral dosage forms can also be used for administering compounds of the invention, including aqueous and non-aqueous solutions, emulsions, suspensions, syrups, and elixirs. Such dosage forms can also contain suitable inert diluents known in the art such as water and suitable excipients known in the art such as preservatives, wetting agents, sweeteners, flavorants, as well as agents for emulsifying and/or suspending the compounds of the invention. The compounds of the present invention may be injected, for example, intravenously, in the form of an isotonic sterile solution. Other preparations are also possible.

Suppositories for rectal administration of the compounds of the present invention can be prepared by mixing the compound with a suitable excipient such as cocoa butter, salicylates and polyethylene glycols. Formulations for vaginal administration can be in the form of a pessary, tampon, cream, gel, paste, foam, or spray formula containing, in addition to the active ingredient, such suitable carriers as are known in the art.

For topical administration the pharmaceutical composition can be in the form of creams, ointments, liniments, lotions, emulsions, suspensions, gels, solutions, pastes, powders, sprays, and drops suitable for administration to the skin, eye, ear or nose. Topical administration may also involve transdermal administration via means such as transdermal patches.

Aerosol formulations suitable for administering via inhalation also can be made. For example, for treatment of disorders of the respiratory tract, the compounds according to the invention can be administered by inhalation in the form of a powder (e.g., micronized) or in the form of atomized solutions or suspensions. The aerosol formulation can be placed into a pressurized acceptable propellant.

The present invention further includes methods of treatment that involve inhibition of PDE4 enzymes. Thus, the present invention includes methods of selective inhibition of PDE4 enzymes in animals, e.g., mammals, especially humans, wherein such inhibition has a therapeutic effect, such as where such inhibition may relieve conditions involving neurological syndromes, such as the loss of memory, especially long-term memory. Such methods comprise administering to a patient in need thereof, especially a mammal, most especially a human, an inhibitory amount of a compound, alone or as part of a formulation, as disclosed herein.

The condition of memory impairment is manifested by impairment of the ability to learn new information and/or the inability to recall previously learned information. Memory impairment is a primary symptom of dementia and can also be a symptom associated with such diseases as Alzheimer's disease, schizophrenia, Parkinson's disease, Huntington's disease, Pick's disease, Creutzfeldt-Jakob disease, HIV, cardiovascular disease, and head trauma as well as age-related cognitive decline.

Dementias are diseases that include memory loss and additional intellectual impairment separate from memory. The present invention includes methods for treating patients suffering from memory impairment in all forms of dementia. Dementias are classified according to their cause and include: neurodegenerative dementias (e.g., Alzheimer's, Parkinson's disease, Huntington's disease, Pick's disease), vascular (e.g., infarcts, hemorrhage, cardiac disorders), mixed vascular and Alzheimer's, bacterial meningitis, Creutzfeldt-Jacob Disease, multiple sclerosis, traumatic (e.g., subdural hematoma or traumatic brain injury), infectious (e.g., HIV), genetic (down syndrome), toxic (e.g., heavy metals, alcohol, some medications), metabolic (e.g., vitamin B12 or folate deficiency), CNS hypoxia, Cushing's disease, psychiatric (e.g., depression and schizophrenia), and hydrocephalus.

The present invention includes methods for dealing with memory loss separate from dementia, including mild cognitive impairment (MCI) and age-related cognitive decline. The present invention includes methods of treatment for memory impairment as a result of disease. In another application, the invention includes methods for dealing with memory loss resulting from the use of general anesthetics, chemotherapy, radiation treatment, post-surgical trauma, and therapeutic intervention.

The compounds of the present invention may be used to treat psychiatric conditions including schizophrenia, bipolar or manic depression, major depression, and drug addiction and morphine dependence. These compounds may enhance wakefulness. PDE4 inhibitors can be used to raise cAMP levels and prevent neurons from undergoing apoptosis. PDE4 inhibitors are also known to be anti-inflammatory. The combination of anti-apoptotic and anti-inflammatory properties make these compounds useful to treat neurodegeneration resulting from any disease or injury, including stroke, spinal cord injury, Alzheimer's disease, multiple sclerosis, amylolaterosclerosis (ALS), and multiple systems atrophy (MSA).

Thus, in accordance with a preferred embodiment, the present invention includes methods of treating patients suffering from memory impairment due to, for example, Alzheimer's disease, multiple sclerosis, amylolaterosclerosis (ALS), multiple systems atrophy (MSA), schizophrenia, Parkinson's disease, Huntington's disease, Pick's disease, Creutzfeldt-Jakob disease, Rubenstein-Taybi syndrome (RSTS), depression, aging, head trauma, stroke, spinal cord injury, CNS hypoxia, cerebral senility, diabetes associated cognitive impairment, memory deficits from early exposure of anesthetic agents, multiinfarct dementia and other neurological conditions including acute neuronal diseases, as well as HIV and cardiovascular diseases, comprising administering an effective amount of a compound according to Formulas I-III or pharmaceutically acceptable salts thereof.

The compounds of the present invention can also be used in a method of treating patients suffering from disease states characterized by decreased NMDA function, such as schizophrenia. The compounds can also be used to treat psychosis characterized by elevated levels of PDE 4, for example, various forms of depression, such as manic depression, major depression, and depression associated with psychiatric and neurological disorders.

The compounds of the present invention can also be used in methods of treating patients suffering from obesity and in treatment methods for neuronal regeneration or neurogenesis.

A subject or patient in whom administration of the therapeutic compound is an effective therapeutic regimen for a disease or disorder is preferably a human, but can be any animal, including a laboratory animal in the context of a clinical trial or screening or activity experiment. Thus, as can be readily appreciated by one of ordinary skill in the art, the methods, compounds and compositions of the present invention are particularly suited to administration to any animal, particularly a mammal, and including, but by no means limited to, humans, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., i.e., for veterinary medical use.

As mentioned, the compounds of the invention also exhibit anti-inflammatory activity. As a result, the inventive compounds are useful in the treatment of a variety of allergic and inflammatory diseases, particularly disease states characterized by decreased cyclic AMP levels and/or elevated phosphodiesterase 4 levels. Thus, in accordance with a further embodiment of the invention, there is provided a method of treating allergic and inflammatory disease states, comprising administering an effective amount of a compound according to Formula I or II, or of the compounds listed above, or a pharmaceutically acceptable salt thereof. Such disease states include: asthma, chronic bronchitis, chronic obstructive pulmonary disease (COPD), atopic dermatitis, urticaria, allergic rhinitis, allergic conjunctivitis, vernal conjunctivitis, esoniophilic granuloma, psoriasis, inflammatory arthritis, rheumatoid arthritis, septic shock, ulcerative colitis, Crohn's disease, reperfusion injury of the myocardium and brain, chronic glomerulonephritis, endotoxic shock, adult respiratory distress syndrome, cystic fibrosis, arterial restenosis, artherosclerosis, keratosis, rheumatoid spondylitis, osteoarthritis, pyresis, diabetes mellitus, pneumoconiosis, chronic obstructive airways disease, chronic obstructive pulmonary disease, toxic and allergic contact eczema, atopic eczema, sebolrheic eczema, lichen simplex, sunburn, pruritis in the anogenital area, alopecia greata, hypertrophic scars, discoid lupus erythematosus, systemic lupus erythematosus, follicular and wide-area pyodermias, endogenous and exogenous acne, acne rosacea, Beghet's disease, anaphylactoid purpura nephritis, inflammatory bowel disease, leukemia, multiple sclerosis, gastrointestinal diseases, autoimmune diseases and the like.

PDE4 inhibitors for treating asthma, chronic bronchitis, psoriasis, allergic rhinitis, and other inflammatory diseases, and for inhibiting tumor necrosis factor are known within the art. See, e.g., WO 98/58901, JP11-18957, JP 10-072415, WO 93/25517, WO 94/14742, U.S. Pat. No. 5,814,651, and U.S. Pat. No. 5,935,978. These references also describe assays for determining PDE4 inhibition activity, and methods for synthesizing such compounds. The entire disclosures of these documents are hereby incorporated by reference.

PDE4 inhibitors may be used to prevent or ameliorate osteoporosis, as an antibiotic, for treatment of cardiovascular disease by mobilizing cholesterol from atherosclerotic lesions, to treat rheumatoid arthritis (RA), for long-term inhibition of mesenchymal-cell proliferation after transplantation, for treatment of urinary obstruction secondary to benign prostatic lyperplasia, for suppression of chemotaxis and reduction of invasion of colon cancer cells, for treatment of B cell chronic lymphocytic leukemia (B-CLL), for inhibition of uterine contractions, to attenuate pulmonary vascular ischemia-reperfusion injury (IRI), for corneal hydration, for inhibition of IL-2R expression and thereby abolishing HIV-1 DNA nuclear import into memory T cells, for augmentation of glucose-induced insulin secretion, in both the prevention and treatment of colitis, and to inhibit mast cell degranulation.

The compounds of the invention are also suitable for use in the treatment of asbestos-related diseases or disorders. See, for example, U.S. Published Application No. 2005/0142104, which is hereby incorporated by reference in its entirety.

Thus, in accordance with a further aspect of the invention, there is provided a method of treating asbestos-related diseases or disorders comprising administering to a patient, such as a mammal, e.g., a human, a therapeutically effective amount of a compound of the invention (e.g., in the form of a pharmaceutically acceptable salt or solvate (e.g., hydrate) thereof). In accordance with a further embodiment, there is provided a method of treating, for example, mesothelioma, asbestosis, pleural effusion, pleural plaque, pleural calcification, diffuse pleural thickening, round atelectasis, and bronchogenic carcinoma, comprising administering to a patient, such as a mammal, e.g., a human, a therapeutically effective amount of a compound of the invention (e.g., in the form of a pharmaceutically acceptable salt or solvate (e.g., hydrate) thereof).

The compounds of the present invention may also be administered in combination with other known therapeutics for the treatment of asbestos-related diseases or disorders including, but not limited to, other PDE-4 inhibitors, anti-cancer agents, antibiotics, anti-inflammatory agents, cytokines, steroids, immunomodulatory agents, immunosuppressive agents, and combinations thereof. In addition, the compounds of the present invention can be used in combination with conventional therapies used to treat, prevent, or manage asbestos-related diseases or disorders, including, but not limited to, chemotherapy, surgery, radiation therapy, photodynamic therapy, and combinations thereof.

When used in combination with one or more additional pharmaceutical agent or agents for the treatment of asbestos-related diseases or disorders, the compounds of the present invention may be administered prior to, concurrently with, or following administration of the additional pharmaceutical agent or agents. When used in combination with one or more conventional therapies for the treatment of asbestos-related diseases or disorders, the compounds of the present invention may be administered prior to, concurrently with, or following the conventional therapy.

The compounds of the invention are also suitable for use in the treatment of psychiatric disorders. See, for example, U.S. Published Application No. 2006/0069115.

Thus, in accordance with a further aspect of the invention, there is provided a method of treating psychiatric disorders comprising administering to a patient, such as a mammal, e.g., a human, a therapeutically effective amount of a compound of the invention (e.g., in the form of a pharmaceutically acceptable salt or solvate (e.g., hydrate) thereof). In accordance with a further embodiment, there is provided a method of treating, for example, fear and anxiety disorders, and mood disorders (for example, panic disorder, phobias, such as specific phobia, posttraumatic stress disorder (PTSD), obsessive-compulsive disorder, and movement disorders such as Tourette's syndrome) comprising administering to a patient, such as a mammal, e.g., a human, a therapeutically effective amount of a compound of the invention (e.g., in the form of a pharmaceutically acceptable salt or solvate (e.g., hydrate) thereof). The disorders contemplated herein are defined in, for example, the DSM-IV (Diagnostic and Statistical Manual; 4th edition, American Psychiatric Association).

According to a further embodiment, there is provided a method of treating psychiatric disorders comprising administering to a patient, such as a mammal, e.g., a human, a therapeutically effective amount of a compound of the invention (e.g., in the form of a pharmaceutically acceptable salt or solvate (e.g., hydrate) thereof) in combination with psychotherapy. This embodiment method comprises subjecting the individual to one or more sessions of a combination therapy protocol, where the combination therapy protocol comprises administering a therapeutically effective amount of a compound of the invention (e.g., in the form of a pharmaceutically acceptable salt or solvate (e.g., hydrate) thereof) in combination with one or more sessions of psychotherapy. Suitable methods of psychotherapy include behavior psychotherapy such as exposure-based psychotherapy, cognitive psychotherapy including cognitive training and psychodynamically oriented psychotherapy (see, for example, Foa, J. Clin. Psych., 61, (suppl. 5), 43-38 (2000)). Exposure based psychotherapy include for example, systematic desensitization, flooding, implosive therapy, and extinction-based therapy. Such psychotherapy modalities are well known to one skilled in the art of psychiatry.

The compounds of Formulas I-III can be administered as the sole active agent or in combination with one or more other pharmaceutical agents such as other agents used in the treatment of cognitive impairment and/or in the treatment of psychosis, e.g., other PDE4 inhibitors, calcium channel blockers, cholinergic drugs, adenosine receptor modulators, ampakines, NMDA-R modulators, mGluR modulators, cholinesterase inhibitors (e.g., donepezil, rivastigimine, and glanthanamine), and selective serotonin reuptake inhibitors (SSRIs). In such combinations, each active ingredient can be administered either in accordance with their usual dosage range or a dose below its usual dosage range.

The compounds of Formulas I-III can be administered as the sole active agent or in combination with one or more other pharmaceutical agents such as other agents used in the treatment of allergic and/or inflammatory conditions, e.g. respiratory conditions. Suitable examples of other pharmaceutical agents which may be used in combination with the compounds of the present invention include, but are not limited to, other PDE-4 inhibitors, 5-lipoxygenase (5-LO) inhibitors or 5-lipoxygenase activating protein (FLAP) antagonists (e.g., zileuton, fenleuton), leukotriene antagonists (LTRAs) including antagonists of LTB₄, LTC₄, LTD₄, and LTE₄ (e.g., ontazolast, ablukast, pranlukast, verlukast, zariflukast, montelukast, zileuton), histaminic receptor antagonists, including H1 and H3 antagonists (e.g., cetirizine, loratidine, desloratidine, fexofenadine, astemizole, azelastine, chlorpheniramine, cimetidine, ranitidine, famotidine, nizatidine), α₁ and α2 adrenoceptor agonist vasoconstrictor sympathomimetic agents for decongestant use (e.g., propylhexedrine, phenylephrine, phenylpropanolamine, pseudoephedrine, naphazoline hydrochloride), muscarinic receptor (M1, M2, and M3) antagonists (e.g., ipratropium salts, namely bromide, tiotropium salts, namely bromide, oxitropium salts, namely bromide, perenzepine, and telenzepine), anticholinergic agents, β₁ to β₄ (e.g. β₂) adrenoceptor agonists (e.g., isoprenaline, albuterol, salbutamol, formoterol, salmeterol), COX-1 inhibitors (NSAIDs), COX-2 selective inhibitors, nitric oxide NSAIDs, oral or inhaled glucocorticosteroids (e.g., prednisone, prednisolone, flunisolide, triamcinolone acetonide, beclomethasone diproprionate), and adenosine A2a receptor agonists. Further examples of suitable other pharmaceutical agents which may be used in combination with the compounds of the present invention are disclosed in U.S. Pat. Nos. 6,559,168 and 6,756,392, which are hereby incorporated by reference in their entireties. In such combinations, each active ingredient can be administered either in accordance with their usual dosage range or a dose below its usual dosage range.

The dosages of the compounds of the present invention depend upon a variety of factors including the particular syndrome to be treated, the severity of the symptoms, the route of administration, the frequency of the dosage interval, the particular compound utilized, the efficacy, toxicology profile, pharmacokinetic profile of the compound, and the presence of any deleterious side-effects, among other considerations.

The compounds of the invention are typically administered at dosage levels and in a manner customary for PDE4 inhibitors such as those known compounds mentioned above. For example, the compounds can be administered, in single or multiple doses, by oral administration at a dosage level of, for example, 0.001-100 mg/kg/day, preferably 0.01-70 mg/kg/day, especially 0.01-10 mg/kg/day. Unit dosage forms can contain, for example, 0.1-50 mg of active compound. For intravenous administration, the compounds can be administered, in single or multiple dosages, at a dosage level of, for example, 0.001-50 mg/kg/day, preferably 0.001-10 mg/kg/day, especially 0.01-1 mg/kg/day. Unit dosage forms can contain, for example, 0.1-10 mg of active compound.

In carrying out the procedures of the present invention it is of course to be understood that reference to particular buffers, media, reagents, cells, culture conditions and the like are not intended to be limiting, but are to be read so as to include all related materials that one of ordinary skill in the art would recognize as being of interest or value in the particular context in which that discussion is presented. For example, it is often possible to substitute one buffer system or culture medium for another and still achieve similar, if not identical, results. Those of skill in the art will have sufficient knowledge of such systems and methodologies so as to be able, without undue experimentation, to make such substitutions as will optimally serve their purposes in using the methods and procedures disclosed herein.

The present invention will now be further described by way of the following non-limiting examples. In applying the disclosure of these examples, it should be kept clearly in mind that other and different embodiments of the methods disclosed according to the present invention will no doubt suggest themselves to those of skill in the relevant art.

In the foregoing and in the following examples, all temperatures are set forth uncorrected in degrees Celsius; and, unless otherwise indicated, all parts and percentages are by weight.

The entire disclosures of all applications, patents and publications, cited above and below, are hereby incorporated by reference.

### EXAMPLES

### Example 1

### 2-Bromo-3-hydroxy-6-iodopyridine

To a mixture of 14 g of 2-bromo-3-hydroxypyridine (80.5 mmol), and 22.3 g of K₂CO₃ (161 mmol) in 180 mL of water at room temperature was added 21.0 g of 12 (82.7 mmol) in one portion. The mixture was stirred at room temperature for 2 h then carefully neutralized with 3N HCl (aq) to pH=6. The solid was collected by vacuum filtration and washed with water (100 mL), 2M aqueous sodium bisulfite (50 mL), and water (100 mL).

The solid was dried in vacuo to give 16.1 g of 2-bromo-3-hydroxy-6-iodopyridine as a tan solid. ¹H NMR (300 Mhz, MeOD) δ 7.57 (d, J=8.3 Hz, 1H), 6.95 (d, J=8.3 Hz, 1H).

### Example 2A

### 2-Bromo-6-iodo-3-methoxypyridine

To a mixture of 16.1 g of 2-bromo-3-hydroxy-6-iodopyridine, and 7.0 g of K₂CO₃ in 35 mL DMF was added 11 mL of iodomethane and the mixture was heated to 100° C. for 2 h. The mixture was cooled and 150 mL of water was added and the solid was collected by vacuum filtration. The solid was washed with several portions of water and dried in vacuo to give 15.7 g of 2-bromo-6-iodo-3-methoxypyridine as a tan solid. ¹H NMR (300 MHz, MeOD) δ 7.70 (d, J=8.3 Hz, 1H), 7.14 (d, J=8.3 Hz, 1H), 3.91 (s, 3H).

### Example 2B

### 2-Bromo-3-difluoromethoxy-6-iodopyridine

To a solution of 5.0 g (16.7 mmol) of 2-bromo-3-hydroxy-6-iodopyridine in 300 mL of DMF was added 7.6 g (50 mmol) of sodium chlorodifluoroacetate and 0.70 g (17.5 mmol) of NaOH. The light brown solution was warmed to 55° C. with stirring for 16 hours, concentrated in vacuo, diluted with 150 mL of H₂O and extracted with 2×150 mL of EtOAc. The combined EtOAc fractions were concentrated to give 4.0 g of crude product which was purified by chromatography over SiO₂ using a gradient elution going from 2% EtOAc in hexanes to 4% EtOAc in hexanes to provide 3.43 g (59% yield) of 2-bromo-3-difluoromethoxy-6-iodopyridine as a pale yellow oil. ¹H NMR (500 MHz, CDCl₃) δ 7.68 (d, J=8.3 Hz, 1H), 7.21 (d, J=8.3 Hz, 1H), 6.58 (t, J=72.0 Hz, 1H), (s, 3H).

### Example 3

### 2-Cyclopentyloxy-6-iodo-3-methoxypyridine

To a mixture of 1.0 g NaH (60% mineral oil dispersion) in 8 mL DMF at room temperature was carefully added 2.2 mL of cyclopentanol and the mixture was allowed to stir for 1 h at room temperature. A solution of 4.95 g of 2-bromo-6-iodo-3-methoxypyridine in DMF (2 mL) was added and the mixture was heated to 100° C. for 2 h. The mixture was cooled to room temperature and partitioned between Et₂O (100 mL) and water (100 mL). The organic layer was separated, washed with brine (50 mL), dried (MgSO₄), and concentrated in vacuo. The residue was purified by column chromatography eluting with a linear gradient from 0% to 10% EtOAc in hexanes to yield 4.0 g of 2-cyclopentyloxy-6-iodo-3-methoxypyridine as a tan solid. ¹H NMR (300 MHz, MeOD) δ 7.18 (d, J=8.1 Hz, 1H), 6.71 (d, J=8.1 Hz, 1H), 5.42 (m, 1H), 3.81 (s, 3H), 2.0-1.8 (m, 2H), 1.8-1.7 (m, 4H), 1.7-1.5 (m, 214).

The following compounds were prepared in a similar manner as described above.
a) 2-Cyclobutyloxy-6-iodo-3-methoxypyridine
b) 2-Cyclopropylmethoxy-6-iodo-3-methoxypyridine
c) 2,3-Dimethoxy-6-iodopyridine
d) 2-Cyclopropylmethoxy-3-difluoromethoxy-6-iodopyridine
e) 3-Difluoromethoxy-6-iodo-2-methoxypyridine
f) 2-Ethoxy-6-iodo-3-methoxypyridine
g) 6-Iodo-2-(2-propyl)oxy-3-methoxypyridine
h) 3-Difluoromethoxy-6-iodo-2-(2-propyl)oxypyridine
i) 2-Cyclobutyloxy-3-difluoromethoxy-6-iodopyridine
j) 6-Iodo-3-methoxy-2-[(3R)-tetrahydrofuran-3-yl]oxypyridine
k) 6-Iodo-3-methoxy-2-[tetrahydrofuran-3-yl]oxypyridine
l) 3-Difluoromethoxy-6-iodo-2-ethoxypyridine

### Example 4

### (3-Chloro-4-methoxy-phenyl)-pyridin-3-ylmethyl-amine

To a mixture of 3-pyridinecarboxaldehyde (2.2 g, 20 mmol) in ethanol (100 mL) was added 3-chloro-4-methoxyaniline (3.14 g, 20 mmol) and p-toluenesulfonic acid monohydrate (2.0 mg). The reaction mixture was stirred for 16 h, cooled to 0° C. and sodium borohydride (0.87 g, 23 mmol) was added portion wise over 4 h. The reaction mixture was slowly warmed to room temperature and stirring continued for 16 hours. The solvent was evaporated and the remaining reaction mixture was diluted with water (50 mL) and extracted with ethyl acetate (2×20 mL). The combined organic layers were washed with brine (5 μL), dried (MgSO₄), and concentrated to yield 2.2 g of (3-Chloro-4-methoxy-phenyl)-pyridin-3-ylmethyl-amine as a solid. ¹H NMR (300 MHz, CDCl₃) δ 8.61 (s, 2H), 8.53 (d, J=4.7 Hz, 1H), 7.68 (d, J=7.8 Hz, 1H), 7.27 (m, 1H), 6.80 (d, J=8.8 Hz, 1H), 6.70 (d, J=2.8 Hz, 1H), 6.48 (dd, J=8.8 Hz, 2.8 Hz, 1H), 4.30 (s, 2H), 3.81 (s, 3H).

The following compounds were prepared, using the appropriate aldehydes and amines in a similar manner as described above. The benzoic acid compounds were prepared using 4-amino-benzoic acid tert-butyl ester, and the ester was subsequently hydrolyzed with TFA.
a) 3-Fluoro-4-difluoromethoxy-N-(3-pyridylmethyl)aniline
b) 4-[(Thiazol-5-ylmethyl)-amino]-benzoic acid
c) 4-[(Oxazol-5-ylmethyl)-amino]-benzoic acid
d) 4-[(Pyrimidin-5-ylmethyl)-amino]-benzoic acid
e) Benzyl-piperidin-4-yl-amine
f) (4-Fluoro-benzyl)-piperidin-4-yl-amine
g) (4-Bromo-benzyl)-piperidin-4-yl-amine

### Example 5

### (3-Chloro-phenyl)-piperidin-4-yl-amine

3-Chloroaniline (10 mL) and Boc-piperidin-4-one (6.0 g) were dissolved in 100 mL methylene chloride and 0.25 mL acetic acid, and the reaction mixture was stirred for 72 hours. The reaction was quenched by addition of dilute NaOH solution, and the resulting mixture was extracted with ethyl acetate. The combined organic layers were dried Na₂SO₄), filtered, and concentrated under reduced pressure. The resulting residue was recrystallized from ethyl acetate/hexanes to afford 3.7 g of (3-chloro-phenyl)-piperidin-4-yl-amine, MS (M+H)=212.

The following compounds were prepared in a similar manner as described above:
a) (4-Methoxy-phenyl)-piperidin-4-yl-amine
b) (4-Cyano-phenyl)-piperidin-4-yl-amine
c) Phenyl-piperidin-4-yl-amine
d) 4-(Piperidin-4-ylamino)-benzoic acid
e) 4-(Piperidin-4-ylamino)-benzamide
f) (4-Methyl-phenyl)-piperidin-4-yl-amine
g) (4-dimethylamino-phenyl)-piperidin-4-yl-amine
h) Piperidin-4-yl-thiophen-3-yl-amine
i) Pyrrolidin-3-yl-p-tolyl-amine

### Example 6

### 4-[[5-(difluoromethoxy)-6-ethoxypyridin-2-yl](1,3-thiazol-5-ylmethyl)amino]benzoic acid

To a 25 mL oven dried, argon flushed flask was added 235 mg 1-Difluoromethoxy-2-ethoxy-4-iodo-benzene (0.75 mmol), 200 mg 4-[(Thiazol-5-ylmethyl)-amino]-benzoic acid tert butyl ester (0.83 mmol), 208 mg of NaOtBu, 35 mg Pd₂ dba₃, and 10 mL of toluene. The mixture was stirred for 18 hours at room temperature, filtered through celite and the celite plug was washed with several portions of toluene, concentrated to 5 mL in vacuo and loaded onto a silica to gel column. The product was eluted using a linear gradient from 45% to 55% EtOAc in hexanes to give 4-[[5-(difluoromethoxy)-6-ethoxypyridin-2-yl](1,3-thiazol-5-ylmethyl)amino]benzoic acid tert-butyl ester, MS (M+H)=479. This ester was taken up in 10 mL of 20% TFA in dichloromethane and stirred overnight. The solvent was removed in vacuo and the residue was partitioned between 50 mL EtOAc and 50 mL water. The aqueous fraction was adjusted to a pH of 5-6 with saturated aqueous sodium bicarbonate and the EtOAc layer was separated, washed with brine, dried and concentrated. The residue was purified by column chromatography eluting with EtOAc to give 4-[[5-(difluoromethoxy)-6-ethoxypyridin-2-yl](1,3-thiazol-5-ylmethyl)amino]benzoic acid, MS (M+H)=422.

The following compounds were prepared in a similar manner as described above:
9) 4-[[5-(difluoromethoxy)-6-ethoxypyridin-2-yl](pyrimidin-5-ylmethyl)amino]benzoic acid, MS (M+H)=417
1) 4-[[6-(cyclopropylmethoxy)-5-(difluoromethoxy)pyridin-2-yl](1,3-thiazol-5-ylmethyl)amino]benzoic acid, MS (M+H)=448
2) 4-[[6-(cyclopropylmethoxy)-5-methoxypyridin-2-yl](1,3-thiazol-5-ylmethyl)amino]benzoic acid, MS (M+H)=412
6) 4-[[5-(difluoromethoxy)-6-ethoxypyridin-2-yl](1,3-oxazol-5-ylmethyl)amino]benzoic acid, MS (M+H)=406
7) 4-[(6-ethoxy-5-methoxypyridin-2-yl)(1,3-thiazol-5-ylmethyl)amino]benzoic acid, MS (M+H)=386
8) 4-[(6-ethoxy-5-methoxypyridin-2-yl)(1,3-oxazol-5-ylmethyl)amino]benzoic acid, MS (M+H)=370
13) 4-[(6-ethoxy-5-methoxypyridin-2-yl)(pyrimidin-5-ylmethyl)amino]benzoic acid, MS (M+H)=381
14) 3-[(6-ethoxy-5-methoxypyridin-2-yl)(pyrimidin-5-ylmethyl)amino]benzoic acid, MS (M+H)=381
11) 4-[[5-(difluoromethoxy)-6-methoxypyridin-2-yl](1,3-thiazol-5-ylmethyl)amino]benzoic acid, MS (M+H)=408

### Example 7

### 5) N-(4-Fluorobenzyl)-6-isopropoxy-5-methoxy-N-piperidin-4-ylpyridin-2-amine

To a 25 mL oven dried, argon flushed flask was added 210 mg 4-(4-Fluoro-benzylamino)-piperidine-1-carboxylic acid tert-butyl ester (0.68 mmol), 210 mg, 200 mg of 4-Iodo-2-isopropoxy-1-methoxy-benzene (0.68 mmol) 20 mg of NaOtBu, 39 mg Pd₂ dba₃, 0.7 mL of 10% tributylphosphine in hexanes, and 7 mL of toluene. The mixture was stirred for 24 hours at 90° C., then cooled, filtered through celite, and concentrated under reduced pressure. The residue was eluted through silica gel using a linear gradient from 45% to 55% EtOAc in hexanes to give 4-[(4-Fluoro-benzyl)-(6-isopropoxy-5-methoxy-pyridin-2-yl)-amino]-piperidine-1-carboxylic acid tert-butyl ester, MS (M+H)=474. This Boc-protected compound was taken up in THF/H₂O/conc HCl (3 mL/1 mL/1 mL) and heated to 60° C. for 15 minutes. The solution was cooled and triturated with isopropanol/diethyl ether to give 166 mg of N-(4-fluorobenzyl)-6-isopropoxy-5-methoxy-N-piperidin-4-ylpyridin-2-amine (M+H)=374.

The following compounds were prepared in a similar manner as described above:
3) N-benzyl-6-(cyclopentyloxy)-5-methoxy-N-piperidin-4-ylpyridin-2-amine, MS (M+H)=382
4) 6-isopropoxy-5-methoxy-N-piperidin-4-yl-N-(1,3-thiazol-5-ylmethyl)pyridin-2-amine, MS (M+H)=363
12) N-(4-bromobenzyl)-6-(cyclopropylmethoxy)-5-methoxy-N-piperidin-4-ylpyridin-2-amine, MS (M+H)=447
26) 6-(cyclopentyloxy)-5-methoxy-N-phenyl-N-piperidin-4-ylpyridin-2-amine, MS (M+H)=368

### Example 8

### 16) N-(3-chlorophenyl)-5-(difluoromethoxy)-6-ethoxy-N-piperidin-4-ylpyridin-2-amine

To a 25 mL oven dried, argon flushed flask was added 200 mg 1-Difluoromethoxy-2-ethoxy-4-iodo-benzene (0.64 mmol) 200 mg, 200 mg of 4-(3-Chloro-benzylamino)-piperidine-1-carboxylic acid tert-butyl ester (0.62 mmol) 210 mg of NaOtBu, 52 mg Pd₂ dba₃, 0.6 mL of 10% tributylphosphine in hexanes, and 15 mL of toluene. The mixture was stirred for 18 hours at 90° C., then cooled, filtered through celite, and concentrated under reduced pressure. The residue was eluted through silica gel using a linear gradient from 45% to 55% EtOAc in hexanes to give 4-[(3-Chloro-phenyl)-(5-difluoromethoxy-6-ethoxy-pyridin-2-yl)-amino]-piperidine-1-carboxylic acid tert-butyl ester, MS (M+H)=499. This Boc-protected compound was taken up in THF/H₂O/conc HCl (3 mL/1 mL/1 mL) and heated to 60° C. for 15 minutes. The solution was cooled and triturated with isopropanol/diethyl ether to give 27 mg of N-(3-chlorophenyl)-5-(difluoromethoxy)-6-ethoxy-N-piperidin-4-ylpyridin-2-amine, (M+H)=398.

The following compounds were prepared in a similar manner as described above:
15) N-(3-chlorophenyl)-6-isopropoxy-5-methoxy-N-piperidin-4-ylpyridin-2-amine, (M+H)=376
17) 5-(difluoromethoxy)-6-methoxy-N-(4-methoxyphenyl)-N-piperidin-4-ylpyridin-2-amine, (M+H)=380
18) 4-[(6-ethoxy-5-methoxypyridin-2-yl)(piperidin-4-yl)amino]benzonitrile, +H)=353
19) 4-[(6-ethoxy-5-methoxypyridin-2-yl)(piperidin-4-yl)amino]benzamide, (M+H)=371
20) 4-[[6-(cyclohexyloxy)-5-methoxypyridin-2-yl](piperidin-4-yl)amino]benzoic acid, (M+H)=426
21) 6-(cyclopropylmethoxy)-5-methoxy-N-phenyl-N-piperidin-3-ylpyridin-2-amine, (M+H)=354
22) 6-ethoxy-5-methoxy-N-piperidin-4-yl-N-3-thienylpyridin-2-amine, (M+H)=334
23) 6-ethoxy-5-methoxy-N-(4-methylphenyl)-N-pyrrolidin-3-ylpyridin-2-amine, (M+H)=328
24) N-(6-isobutoxy-5-methoxypyridin-2-yl)-N′,N′-dimethyl-N-piperidin-4-ylbenzene-1,4-diamine, (M+H)=399
25) N-(3-chlorophenyl)-N-[4-(difluoromethoxy)-3-methoxyphenyl]piperidin-4-amine, (M+H)=383

### Example 9

### In Vitro Measurement of Type 4 Phosphodiesterase Inhibition Activity

Human PDE4 was obtained from baculovirus-infected Sf9 cells that expressed the recombinant enzyme. The cDNA encoding hPDE-4D6 was subcloned into a baculovirus vector.

Insect cells (Sf9) were infected with the baculovirus and cells were cultured until protein was expressed. The baculovirus-infected cells were lysed and the lysate was used as source of hPDE-4D6 enzyme. The enzyme was partially purified using a DEAE ion exchange chromatography. This procedure can be repeated using cDNA encoding other PDE-4 enzymes.

### Assay:

Type 4 phosphodiesterases convert cyclic adenosine monophosphate (cAmP) to 5′-adenosine monophosphate (5′-AMP). Nucleotidase converts 5′-AMP to adenosine. Therefore the combined activity of PDE4 and nucleotidase converts cAMP to adenosine. Adenosine is readily separated from cAMP by neutral alumina columns. Phosphodiesterase inhibitors block the conversion of cAMP to adenosine in this assay; consequently, PDE4 inhibitors cause a decrease in adenosine.

Cell lysates (40 ul) expressing hPDE-4D6 were combined with 50 ul of assay mix and 110 μl of inhibitors and incubated for 12 min at room temperature. Final concentrations of assay components were: 0.4 ug enzyme, 10 mM Tris-HCl (pH 7.5), 1 mM MgCl₂, 3 uM cAMP, 0.002 U 5′-nucleotidase, and 3×10⁴ cpm of [3H]cAMP. The reaction was stopped by adding 100 μl of boiling 5 mN HCl. An aliquot of 75 μl of reaction mixture was transferred from each well to alumina columns (Multiplate; Millipore). Labeled adenosine was eluted into an OptiPlate by spinning at 2000 rpm for 2 min; 150 μl per well of scintillation fluid was added to the OptiPlate. The plate was sealed, shaken for about 30 min, and cpm of [³H]adenosine was determined using a Wallac Triflux®.

All test compounds are dissolved in 100% DMSO and diluted into the assay such that the final concentration of DMSO is 0.1%. DMSO does not affect enzyme activity at this concentration.

A decrease in adenosine concentration is indicative of inhibition of PDE activity. pIC₅₀ values were determined by screening 6 to 12 concentrations of compound ranging from 0.1 nM to 10,000 nM and then plotting drug concentration versus ³H-adenosine concentration. Nonlinear regression software (Assay Explorer®) was used to estimate pIC₅₀ values.

IC₅₀ values for the preferred compounds of the invention are less than 1000 nM, especially less than 100 μM.

### Example 10

### Method A

### Passive Avoidance in Rats, an In Vivo Test for Learning and Memory

The test was performed as previously described (Zhang, H.-T., Crissman, A. M., Dorairaj, N. R., Chandler, L. J., and O'Donnell, J. M., Neuropsychopharmacology, 2000, 23, 198-204.). The apparatus (Model E10-16SC, Coulbourn Instruments, Allentown, Pa.) consisted of a two-compartment chamber with an illuminated compartment connected to a darkened compartment by a guillotine door. The floor of the darkened compartment consisted of stainless steel rods through which an electric foot-shock could be delivered from a constant current source. All experimental groups were first habituated to the apparatus the day before the start of the experiment. During the training, the rat (Male Spraque-Dawley (Harlan) weighing 250 to 350 g) was placed in the illuminated compartment facing away from the closed guillotine door for 1 minute before the door was raised. The latency for entering the darkened compartment was recorded. After the rat entered the darkened compartment, the door was closed and a 0.5 nA electric shock was administered for 3 seconds. Twenty-four hours later, the rat was administered 0.1 mg/kg MK-801 or saline, 30 minutes prior to the injection of saline or test compound (dosed from 0.1 to 2.5 mg/kg, i.p.), which was 30 minutes before the retention test started. The rat was again placed in the illuminated compartment with the guillotine door open. The latency for entering the darkened compartment was recorded for up to 180 seconds, at which time the trial was terminated.

All data were analyzed by analyses of variance (ANOVA); individual comparisons were made using Kewman-Keuls tests. Naïve rats required less than 30 seconds, on average, to cross from the illuminated compartment to the darkened compartment. However, 24 hours after the electric shock exposure, most rats pretreated with vehicle did not re-enter the darkened compartment; the average latency was increased up to 175 seconds (p<0.001). Pretreatment with NK-801 (0.1 mg/kg) markedly reduced this latency when compared to the vehicle (p<0.001). This amnesic effect of MK-801 is reversed in a statistically significant manner by actual test compounds in a dose-dependent fashion.

### Example 11

### Method B

### Radial Arm Maze Task in Rats, an In Vivo Test for Learning and Memory

The test was performed as previously described (Zhang, H.-T., Crissman, A. M., Dorairaj, N. R., Chandler, L. J., and O'Donnell, J. M., Neuropsychopharmacology, 2000, 23, 198-204.). Five days after initial housing, rats (male Spraque-Dawley (Harlan) weighing 250 to 350 g) were placed in the eight-arm radial maze (each arm was 60×10×12 cm high; the maze was elevated 70 cm above the floor) for acclimation for two days. Rats were then placed individually in the center of the maze for 5 minutes with food pellets placed close to the food wells, and then, the next day, in the wells at the end of the arms; 2 sessions a day were conducted. Next, four randomly selected arms were then baited with one pellet of food each. The rat was restricted to the center platform (26 cm in diameter) for 15 seconds and then allowed to move freely throughout the maze until it collected all pellets of food or 10 minutes passed, whichever came first.

Four parameters were recorded: 1) working memory errors, i.e., entries into baited arms that had already been visited during the same trial; 2) reference memory errors, i.e., entries into unbaited arms; 3) total arm entries; and 4) the test duration (seconds), i.e., the time spent in the collection of all the pellets in the maze. If the working memory error was zero and the average reference memory error was less than one in five successive trials, the rats began the drug tests. MK-801 or saline was injected 15 minutes prior to vehicle or test agent, which was given 45 minutes before the test. Experiments were performed in a lighted room, which contained several extra-maze visual cues.

All data were analyzed by analyses of variance (ANOVA); individual comparisons were made using Kewman-Keuls tests. Compared to control, MK-801 (0.1 mg/kg, i.p.) increased the frequencies of both working and reference memory errors (p<0.01). This amnesic effect of MK-801 on working memory is reversed in a statistically significant manner by the administration of actual test compounds in a dose-dependent fashion.

The preceding examples can be repeated with similar success by substituting the generically or specifically described reactants and/or operating conditions of this invention for those used in the preceding examples.

While the invention has been illustrated with respect to the production and of particular compounds, it is apparent that variations and modifications of the invention can be made without departing from the spirit or scope of the invention.

1. A compound according to Formula I: 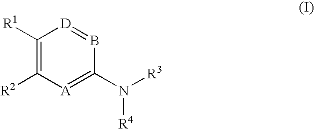
wherein
A, B and D are each, independently, N or CR⁵ wherein at least one of A, B and D is N;
R¹ is halogen, alkyl having 1 to 4 carbon atoms, halogenated alkyl having 1 to 4 carbon atoms, OR⁶, COR⁶, CONR⁶R¹⁰, or NR⁶COR¹⁰;
R² is halogen, alkyl having 1 to 4 carbon atoms, halogenated alkyl having 1 to 4 carbon atoms, OR⁷, COR⁶, CONR⁶R¹⁰, or NR⁶COR¹⁰;
R³ is a partially unsaturated carbocycle-alkyl group wherein the carbocyclic portion has 5 to 14 carbon atoms and the alkyl portion which is branched or unbranched has 1 to 5 carbon atoms, wherein the partially unsaturated carbocycle-group is unsubstituted, substituted in the carbocyclic portion one or more times by halogen, alkyl, alkoxy, nitro, cyano, oxo, or combinations thereof, and/or substituted in the alkyl portion one or more times by halogen, C₁₋₄-alkoxy, cyano or combinations thereof,
arylalkyl having 7 to 19 carbon atoms, wherein the aryl portion has 6 to 14 carbon atoms and the alkyl portion, which is branched or unbranched, has 1 to 5 carbon atoms, wherein the arylalkyl radical is unsubstituted or substituted, in the aryl portion, one or more times by halogen, trifluoromethyl, CF₃O, nitro, amino, alkyl, alkoxy, amino, alkylamino, dialkylamino, or combinations thereof, and/or substituted in the alkyl portion by halogen, cyano, alkyl having 1 to 4 carbon atoms, or combinations thereof, wherein in the alkyl portion one or more —CH₂CH₂— groups are each optionally replaced by —CH═CH— or —C≡C—, and/or one or more —CH₂— groups are each optionally replaced by —O— or —NH—, or
a heterocycle-alkyl group, wherein the heterocyclic portion is saturated, partially saturated or unsaturated, and has 5 to 10 ring atoms in which at least 1 ring atom is an N,N—O, O or S, the alkyl portion, which is branched or unbranched, has 1 to 5 carbon atoms, the heterocycle-alkyl group is unsubstituted, substituted one or more times in the heterocyclic portion by halogen, alkyl, alkoxy, cyano, trifluoromethyl, CF₃O, nitro, oxo, amino, alkylamino, dialkylamino, or combinations thereof and/or substituted in the alkyl portion one or more times by halogen, cyano, alkyl having 1 to 4 carbon atoms, or combinations thereof;
R⁴ is cycloalkyl having 3 to 10 carbon atoms, which is unsubstituted or substituted one or more times by halogen, hydroxy, oxo, cyano, alkyl having 1 to 4 carbon atoms, alkoxy having 1 to 4 carbon atoms, or combinations thereof,
aryl having 6 to 14 carbon atoms and which is unsubstituted or substituted one or more times by halogen, alkyl, alkenyl, alkynyl, hydroxy, alkoxy, alkoxyalkoxy, nitro, methylenedioxy, ethylenedioxy, trifluoromethyl, OCF₃, amino, aminoalkyl, aminoalkoxy, dialkylamino, hydroxyalkyl, hydroxamic acid, pyrrolyl, tetrazole-5-yl, 2(-heterocycle)tetrazole-5-yl, hydroxyalkoxy, carboxy, carboxyalkyl, alkoxycarbonyl, cyano, acyl, alkylthio, alkylsulfinyl, alkylsulfonyl, phenoxy, trialkylsilyloxy, R⁸-L-, or combinations thereof,
heteroaryl having 5 to 10 ring atoms in which at least 1 ring atom is a heteroatom, which is unsubstituted or substituted one or more times by halogen, alkyl, hydroxy, alkoxy, alkoxyalkoxy, nitro, methylenedioxy, ethylenedioxy, trifluoromethyl, amino, aminomethyl, aminoalkyl, aminoalkoxy, dialkylamino, hydroxyalkyl, hydroxamic acid, tetrazole-5-yl, hydroxyalkoxy, carboxy, carboxyalkyl, alkoxycarbonyl, cyano, acyl, alkylthio, alkylsulfinyl, alkylsulfonyl, phenoxy, trialkylsilyloxy, R⁸-L-, or combinations thereof,
a heterocyclic group, which is saturated or partially saturated, having 5 to 10 ring atoms in which at least 1 ring atom is an N, O or S atom, which is unsubstituted or substituted one or more times by halogen, alkyl, hydroxy, alkoxy, alkoxyalkoxy, nitro, oxo, methylenedioxy, ethylenedioxy, trifluoromethyl, OCF₃, amino, aminomethyl, aminoalkyl, aminoalkoxy, dialkylamino, hydroxyalkyl, hydroxamic acid, tetrazole-5-yl, hydroxyalkoxy, carboxy, alkoxycarbonyl, cyano, acyl, alkylthio, alkylsulfinyl, alkylsulfonyl, phenylsulfonyl, phenoxy, cycloalkyl, aryl, heteroaryl or combinations thereof,
a heterocycle-alkyl group, wherein the heterocyclic portion is saturated, partially saturated or unsaturated, and has 5 to 10 ring atoms in which at least 1 ring atom is an N, O or S atom, and the alkyl portion is branched or unbranched and has 1 to 5 carbon atoms, the heterocycle-alkyl group is unsubstituted, substituted one or more times in the heterocyclic portion by halogen, alkyl, hydroxy, alkoxy, alkoxyalkoxy, nitro, oxo, methylenedioxy, ethylenedioxy, trifluoromethyl, OCF₃, amino, aminomethyl, aminoalkyl, aminoalkoxy, dialkylamino, hydroxyalkyl, hydroxamic acid, tetrazole-5-yl, hydroxyalkoxy, carboxy, alkoxycarbonyl, cyano, acyl alkylthio, alkylsulfinyl, alkylsulfonyl, phenylsulfonyl, phenoxy, cycloalkyl, aryl, heteroaryl or combinations thereof, and/or substituted in the alkyl portion one or more times by halogen, oxo, hydroxy, cyano, or combinations thereof, and wherein in the alkyl portion one or more —CH₂CH₂— groups are each optionally replaced by —CH═CH— or —C≡C—, and one or more —CH₂— groups are each optionally replaced by —O— or —NH—;
R⁵ is H, halogen, alkyl having 1 to 4 carbon atoms, halogenated alkyl having 1 to 4 carbon atoms, alkoxy having 1 to 4 carbon atoms, or halogenated alkoxy having 1 to 4 carbon atoms;
R⁶ is H or alkyl having 1 to 4 carbon atoms, which is branched or unbranched and which is unsubstituted or substituted one or more times by halogen;
R⁷ is H or alkyl having 1 to 12 carbon atoms, which is branched or unbranched and which is unsubstituted or substituted one or more times by halogen, hydroxy, cyano, C₁₋₄-alkoxy, oxo or combinations thereof, and wherein optionally one or more —CH₂CH₂— groups is replaced in each case by —CH═CH— or —C≡C—,
cycloalkyl having 3 to 10 carbon atoms, which is unsubstituted or substituted one or more times by halogen, hydroxy, oxo, cyano, alkyl having 1 to 4 carbon atoms, alkoxy having 1 to 4 carbon atoms, or combinations thereof,
cycloalkylalkyl having 4 to 16 carbon atoms, which is unsubstituted or substituted in the cycloalkyl portion and/or the alkyl portion one or more times by halogen, oxo, cyano, hydroxy, C₁₋₄-alkyl, C₁₋₄-alkoxy or combinations thereof,
aryl having 6 to 14 carbon atoms, which is unsubstituted or substituted one or more times by halogen, CF₃, OCF₃, alkyl, hydroxy, alkoxy, nitro, methylenedioxy, ethylenedioxy, cyano, or combinations thereof,
arylalkyl in which the aryl portion has 6 to 14 carbon atoms and the alkyl portion, which is branched or unbranched, has 1 to 5 carbon atoms, wherein the arylalkyl radical is unsubstituted, substituted in the aryl portion one or more times by halogen, CF₃, OCF₃, alkyl, hydroxy, alkoxy, nitro, cyano, methylenedioxy, ethylenedioxy, or combinations thereof, and/or substituted in the alkyl portion one or more times by halogen, oxo, hydroxy, cyano, or combinations thereof, and wherein in the alkyl portion one or more —CH₂CH₂— groups are each optionally replaced by —CH═CH— or —C≡C—, and one or more —CH₂— groups are each optionally replaced by —O— or —NH—,
a partially unsaturated carbocyclic group having 5 to 14 carbon atoms, which is unsubstituted or substituted one or more times by halogen, alkyl, alkoxy, hydroxy, nitro, cyano, oxo, or combinations thereof,
a heterocyclic group, which is saturated, partially saturated or unsaturated, having 5 to 10 ring atoms in which at least 1 ring atom is an N, O or S atom, which is unsubstituted or substituted one or more times by halogen, hydroxy, aryl, alkyl, alkoxy, cyano, trifluoromethyl, nitro, oxo, or combinations thereof, or
a heterocycle-alkyl group, wherein the heterocyclic portion is saturated, partially saturated or unsaturated, and has 5 to 10 ring atoms in which at least 1 ring atom is an N, O or S atom, and the alkyl portion is branched or unbranched and has 1 to 5 carbon atoms, the heterocycle-alkyl group is unsubstituted, substituted one or more times in the heterocyclic portion by halogen, OCF₃, hydroxy, aryl, alkyl, alkoxy, cyano, trifluoromethyl, nitro, oxo, or combinations thereof, and/or substituted in the alkyl portion one or more times by halogen, oxo, hydroxy, cyano, or combinations thereof, and wherein in the alkyl portion one or more —CH₂CH₂— groups are each optionally replaced by —CH═CH— or —C≡C—, and one or more —CH₂— groups are each optionally replaced by —O— or —NH—;
R⁸ is H,
alkyl having 1 to 8 carbon atoms, which is unsubstituted or substituted one or more times by halogen, C₁₋₄-alkyl, C₁₋₄-alkoxy, oxo, or combinations thereof,
alkylamino or dialkylamino wherein each alkyl portion has independently 1 to 8 carbon atoms,
a partially unsaturated carbocycle-alkyl group wherein the carbocyclic portion has 5 to 14 carbon atoms and the alkyl portion has 1 to 5 carbon atoms, and which is unsubstituted or substituted one or more times by halogen, alkyl, alkoxy, nitro, cyano, oxo, or combinations thereof,
cycloalkyl having 3 to 10 carbon atoms, which is unsubstituted or substituted one or more times by halogen, hydroxy, oxo, cyano, alkoxy, alkyl having 1 to 4 carbon atoms, or combinations thereof,
cycloalkylalkyl having 4 to 16 carbon atoms, which is unsubstituted or substituted in the cycloalkyl portion and/or the alkyl portion one or more times by halogen, oxo, cyano, hydroxy, alkyl, alkoxy or combinations thereof,
aryl having 6 to 14 carbon atoms which is unsubstituted or substituted one or more times by halogen, alkyl, hydroxy, alkoxy, alkoxyalkoxy, nitro, methylenedioxy, ethylenedioxy, trifluoromethyl, amino, aminomethyl, aminoalkyl, aminoalkoxy, dialkylamino, hydroxyalkyl, hydroxamic acid, tetrazole-5-yl, hydroxyalkoxy, carboxy, alkoxycarbonyl, cyano, acyl, alkylthio, alkylsulfinyl, alkylsulfonyl, phenoxy, cycloalkyl, aryl, heteroaryl or combinations thereof,
arylalkyl having 7 to 19 carbon atoms, wherein the aryl portion has 6 to 14 carbon atoms and the alkyl portion, which is branched or unbranched, has 1 to 5 carbon atoms, wherein the arylalkyl radical is unsubstituted or substituted, in the aryl portion, one or more times by halogen, trifluoromethyl, CF₃O, nitro, amino, alkyl, alkoxy, amino, alkylamino, dialkylamino, or combinations thereof, and/or substituted in the alkyl portion by halogen, cyano, alkyl having 1 to 4 carbon atoms, or combinations thereof, wherein in the alkyl portion one or more —CH₂CH₂— groups are each optionally replaced by —CH═CH— or —C≡C—, and/or one or more —CH₂— groups are each optionally replaced by —O— or —NH—,
a heterocyclic group, which is saturated, partially saturated or unsaturated, having 5 to 10 ring atoms in which at least 1 ring atom is an N, O or S atom, which is unsubstituted or substituted one or more times by halogen, alkyl, hydroxy, alkoxy, alkoxyalkoxy, nitro, methylenedioxy, ethylenedioxy, trifluoromethyl, amino, aminomethyl, aminoalkyl, aminoalkoxy, dialkylamino, hydroxyalkyl, hydroxamic acid, tetrazole-5-yl, hydroxyalkoxy, carboxy, alkoxycarbonyl, cyano, acyl, alkylthio, alkylsulfinyl, alkylsulfonyl, phenoxy, cycloalkyl, aryl, heteroaryl or combinations thereof, or
a heterocycle-alkyl group, wherein the heterocyclic portion is saturated, partially saturated or unsaturated, and has 5 to 10 ring atoms in which at least 1 ring atom is an N, O or S atom, and the alkyl portion, which is branched or unbranched, has 1 to 5 carbon atoms, the heterocycle-alkyl group is unsubstituted, substituted one or more times in the heterocyclic portion by halogen, alkyl, alkoxy, cyano, trifluoromethyl, CF₃O, nitro, oxo, amino, alkylamino, dialkylamino, or combinations thereof and/or substituted one or more times in the alkyl portion by halogen, cyano, alkyl having 1 to 4 carbon atoms, or combinations thereof;
L is a single bond or a divalent aliphatic radical having 1 to 8 carbon atoms wherein one or more —CH₂— groups are each optionally replaced by —O—, —S—, —SO—, —SO₂—, NR⁹—, —SO₂NR⁹—, —NR⁹SO₂—, —CO—, —CO₂—, —NR⁹CO—, —CONR⁹—, —NHCONH—, —OCONH, —NHCOO—, —SCONH—, —SCSNH—, —NHCSNH—, —CONHSO₂— or —SO₂NHCO—; and
R⁹ is H,
alkyl having 1 to 8 carbon atoms, which is branched or unbranched and which is unsubstituted or substituted one or more times with halogen, C₁₋₄-alkyl, C₁₋₄-alkoxy, oxo, or combinations thereof,
arylalkyl having 7 to 19 carbon atoms, wherein the aryl portion has 6 to 14 carbon atoms and the alkyl portion, which is branched or unbranched, has 1 to 5 carbon atoms, wherein the arylalkyl radical is unsubstituted or substituted, in the aryl portion, one or more times by halogen, trifluoromethyl, CF₃O, nitro, amino, alkyl, alkoxy, amino, alkylamino, dialkylamino, or combinations thereof, and/or substituted in the alkyl portion by halogen, cyano, alkyl having 1 to 4 carbon atoms, or combinations thereof, wherein in the alkyl portion one or more —CH₂CH₂— groups are each optionally replaced by —CH═CH— or —C≡C—, and/or one or more —CH₂— groups are each optionally replaced by —O— or —NH—, or
aryl having 6 to 14 carbon atoms and which is unsubstituted or substituted one or more times by halogen, alkyl, hydroxy, alkoxy, alkoxyalkoxy, nitro, methylenedioxy, ethylenedioxy, trifluoromethyl, amino, aminomethyl, aminoalkyl, aminoalkoxy dialkylamino, hydroxyalkyl, hydroxamic acid, tetrazole-5-yl, hydroxyalkoxy, carboxy, alkoxycarbonyl, cyano, acyl, alkylthio, alkylsulfinyl, alkylsulfonyl, or combinations thereof; and
R¹⁰ is H or alkyl having 1 to 4 carbon atoms, which is branched or unbranched and which is unsubstituted or substituted one or more times by halogen; or
a pharmaceutically acceptable salt or solvates thereof, or a solvate of a pharmaceutically acceptable salt thereof;
wherein if the compound exhibits chirality it can be in the form of a mixture of enantiomers such as a racemate or a mixture of diastereomers, or can be in the form of a single enantiomer or a single diastereomer;
with the provisos that:
when R³ is pyridinylmethyl, R⁴ is other than substituted or unsubstituted piperidinyl, substituted or unsubstituted phenyl, or cyclohexyl, and
wherein said compound is not:
4-[[[5-methoxy-6-[[tetrahydro-3-furanyl]oxy]-2-pyridinyl](3-pyridinylmethyl)amino]methyl]-1-piperidinecarboxylic acid, 1,2-dimethylethyl ester,
N-[5-methoxy-6-[[tetrahydro-3-furanyl]oxy]-2-pyridinyl]-N-(4-piperidinylmethyl)-3-pyridinemethanamine,
4-[[[3,5-bis(trifluoromethyl)phenyl]methyl][5-bromo-4(phenylmethoxy)-2-pyrimidinyl]amino]-2-ethyl-3,4-dihydro-6-methoxy-1,5-naphthydrine-1(2H)-carboxylic acid ethyl ester,
5-chloro-N-(3-chlorophenyl)-4,6-difluoro-N-(4-methoxybenzyl)pyrimidin-2-amine,
or a pharmaceutically acceptable salt thereof, or solvate thereof, or solvate of a pharmaceutically acceptable salt thereof.

2. (canceled)

3. A compound according to claim 1, wherein R¹ is alkyl having 1 to 4 carbon atoms, OR⁶, COR⁶, CONR⁶R¹⁰, or NR⁶COR¹⁰; R² is alkyl having 1 to 4 carbon atoms, OR⁷, COR⁶, CONR⁶R¹⁰, or NR⁶COR¹⁰; R⁵ is H, alkyl having 1 to 4 carbon atoms, or alkoxy having 1 to 4 carbon atoms; and R⁴ is cycloalkyl, aryl, heteroaryl, or a heterocyclic group, which in each case is unsubstituted or substituted.

4. A compound according to claim 1, wherein B is CR⁵.

5. A compound according to claim 1, wherein D is CR⁵.

6. A compound according to claim 1, wherein one of A, B, and D is N and the others are CR⁵.

7. A compound according to claim 6, wherein one of A, B, and D is N and the others are CH.

8. (canceled)

9. A compound according to claim 1, wherein R⁴ is aryl or a heterocyclic group, which in each case is unsubstituted or substituted.

10. A compound according to claim 1, wherein R³ is an arylalkyl or a heteroarylalkyl group, other than pyridinylmethyl, which in each case is unsubstituted or substituted.

11. A compound according to claim 1, wherein one of A, B, and D is N and the others are CR⁵; R¹ is OR⁶; R² is OR⁷; R³ is an arylalkyl or a heteroarylalkyl group, other than pyridinylmethyl, which is in each case unsubstituted or substituted; R⁴ is aryl or a heterocyclic group, which is in each case substituted or unsubstituted; R⁶ is alkyl, or halogenated alkyl; and R⁷ is alkyl, halogenated alkyl, cycloalkyl, cycloalkylalkyl, or a heterocyclic group; and wherein if R³ is prymidinylmethyl, then R⁴ is not piperidinyl or aryl.

12. A compound according to claim 1, wherein said compound is selected from:
4-[[6-(Cyclopropylmethoxy)-5-(difluoromethoxy)pyridin-2-yl](1,3-thiazol-5-ylmethyl)amino]benzoic acid,
4-[[6-(Cyclopropylmethoxy)-5-methoxypyridin-2-yl](1,3-thiazol-5-ylmethyl)amino]benzoic acid,
N-Benzyl-6-(cyclopentyloxy)-5-methoxy-N-piperidin-4-ylpyridin-2-amine,
6-Isopropoxy-5-methoxy-N-piperidin-4-yl-N-(1,3-thiazol-5-ylmethyl)pyridin-2-amine,
N-(4-Fluorobenzyl)-6-isopropoxy-5-methoxy-N-piperidin-4-ylpyridin-2-amine,
4-[[5-(Difluoromethoxy)-6-ethoxypyridin-2-yl](1,3-oxazol-5-ylmethyl)amino]benzoic acid,
4-[(6-Ethoxy-5-methoxypyridin-2-yl)(1,3-thiazol-5-ylmethyl)amino]benzoic acid,
4-[(6-Ethoxy-5-methoxypyridin-2-yl)(1,3-oxazol-5-ylmethyl)amino]benzoic acid,
4-[[5-(Difluoromethoxy)-6-ethoxypyridin-2-yl](pyrimidin-5-ylmethyl)amino]benzoic acid,
4-[[5-(Difluoromethoxy)-6-ethoxypyridin-2-yl](1,3-thiazol-5-ylmethyl)amino]benzoic acid,
4-[[5-(Difluoromethoxy)-6-methoxypyridin-2-yl](1,3-thiazol-5-ylmethyl)amino]benzoic acid,
N-(4-Bromobenzyl)-6-(cyclopropylmethoxy)-5-methoxy-N-piperidin-4-ylpyridin-2-amine,
4-[(6-Ethoxy-5-methoxypyridin-2-yl)(pyrimidin-5-ylmethyl)amino]benzoic acid, and
3-[(6-Ethoxy-5-methoxypyridin-2-yl)(pyrimidin-5-ylmethyl)amino]benzoic acid;
and pharmaceutically acceptable salts thereof, solvates thereof, and solvates of pharmaceutically acceptable salts thereof;
wherein if the compound exhibits chirality it can be in the form of a mixture of enantiomers such as a racemate or a mixture of diastereomers, or can be in the form of a single enantiomer or a single diastereomer.

13. A compound according to claim 12, wherein said compound is in the form of a hydrochloride, an oxalate, a hydroformate or a trifluoroacetate salt.

14. A compound according Formula II: 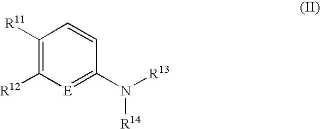
wherein
E is N or CR⁵;
R¹¹ is halogen, alkyl having 1 to 4 carbon atoms, halogenated alkyl having 1 to 4 carbon atoms, OR¹⁶, COR¹⁶, CONHR¹⁶R²⁰, or NR¹⁶COR²⁰;
R¹² is halogen, alkyl having 1 to 4 carbon atoms, halogenated alkyl having 1 to 4 carbon atoms, OR¹⁷, COR¹⁶, CONHR¹⁶R²⁰, or NR¹⁶COR²⁰,
R¹³ a non-aromatic heterocyclic group, which is fully saturated or partially saturated, having 5 to 10 ring atoms in which at least 1 ring atom is an N, O or S atom, which is unsubstituted or substituted one or more times by halogen, alkyl, hydroxy, alkoxy, alkoxyalkoxy, hydroxyalkyl-alkoxy, dihydroxyalkyl-alkoxy, nitro, oxo, methylenedioxy, ethylenedioxy, trifluoromethyl, OCF₃, amino, aminomethyl, aminoalkyl, aminoalkoxy, dialkylamino, hydroxyalkyl, dihydroxyalkyl, hydroxamic acid, tetrazole-5-yl, hydroxyalkoxy, carboxy, alkoxycarbonyl, cyano, acyl, alkylthio, alkylsulfinyl, alkylsulfonyl, phenylsulfonyl, phenoxy, cycloalkyl, aryl, heteroaryl or combinations thereof,
R¹⁴ is cycloalkyl having 3 to 10 carbon atoms, which is unsubstituted or substituted one or more times by halogen, hydroxy, oxo, cyano, alkyl having 1 to 4 carbon atoms, alkoxy having 1 to 4 carbon atoms, or combinations thereof,
aryl having 6 to 14 carbon atoms and which is unsubstituted or substituted one or more times by halogen, alkyl, alkenyl, alkynyl, hydroxy, alkoxy, alkoxyalkoxy, nitro, methylenedioxy, ethylenedioxy, trifluoromethyl, OCF₃, amino, aminoalkyl, aminoalkoxy, dialkylamino, amido, hydroxyalkyl, hydroxamic acid, pyrrolyl, tetrazole-5-yl, 2(-heterocycle)tetrazole-5-yl, hydroxyalkoxy, carboxy, carboxyalkyl, alkoxycarbonyl, cyano, acyl, alkylthio, alkylsulfinyl, alkylsulfonyl, phenoxy, trialkylsilyloxy, R¹⁸-M-, or combinations thereof,
heteroaryl having 5 to 10 ring atoms in which at least 1 ring atom is a heteroatom, which is unsubstituted or substituted one or more times by halogen, alkyl, hydroxy, alkoxy, alkoxyalkoxy, nitro, methylenedioxy, ethylenedioxy, trifluoromethyl, amino, aminomethyl, aminoalkyl, aminoalkoxy, dialkylamino, hydroxyalkyl, hydroxamic acid, tetrazole-5-yl, hydroxyalkoxy, carboxy, carboxyalkyl, alkoxycarbonyl, cyano, acyl, alkylthio, alkylsulfinyl, alkylsulfonyl, phenoxy, trialkylsilyloxy, R¹⁸-M-, or combinations thereof,
a heterocyclic group, which is saturated or partially saturated, having 5 to 10 ring atoms in which at least 1 ring atom is an N, O or S atom, which is unsubstituted or substituted one or more times by halogen, alkyl, hydroxy, alkoxy, alkoxyalkoxy, nitro, oxo, methylenedioxy, ethylenedioxy, trifluoromethyl, OCF₃, amino, aminomethyl, aminoalkyl, aminoalkoxy, dialkylamino, hydroxyalkyl, hydroxamic acid, tetrazole-5-yl, hydroxyalkoxy, carboxy, alkoxycarbonyl, cyano, acyl, alkylthio, alkylsulfinyl, alkylsulfonyl, phenylsulfonyl, phenoxy, cycloalkyl, aryl, heteroaryl or combinations thereof, and
a heterocycle-alkyl group, wherein the heterocyclic portion is saturated, partially saturated or unsaturated, and has 5 to 10 ring atoms in which at least 1 ring atom is an N, O or S atom, and the alkyl portion is branched or unbranched and has 1 to 5 carbon atoms, the heterocycle-alkyl group is unsubstituted, substituted one or more times in the heterocyclic portion by halogen, alkyl, hydroxy, alkoxy, alkoxyalkoxy, nitro, oxo, methylenedioxy, ethylenedioxy, trifluoromethyl, OCF₃, amino, aminomethyl, aminoalkyl, aminoalkoxy, dialkylamino, hydroxyalkyl, hydroxamic acid, tetrazole-5-yl, hydroxyalkoxy, carboxy, alkoxycarbonyl, cyano, acyl alkylthio, alkylsulfinyl, alkylsulfonyl, phenylsulfonyl, phenoxy, cycloalkyl, aryl, heteroaryl or combinations thereof, and/or substituted in the alkyl portion one or more times by halogen, oxo, hydroxy, cyano, or combinations thereof, and wherein in the alkyl portion one or more —CH₂CH₂— groups are each optionally replaced by —CH═CH— or —C≡C—, and one or more —CH₂— groups are each optionally replaced by —O— or —NH—;
R¹⁵ is H, halogen, alkyl having 1 to 4 carbon atoms, halogenated alkyl having 1 to 4 carbon atoms, alkoxy having 1 to 4 carbon atoms, or halogenated alkoxy having 1 to 4 carbon atoms;
R¹⁶ is H or alkyl having 1 to 4 carbon atoms, which is branched or unbranched and which is unsubstituted or substituted one or more times by halogen;
R¹⁷ is H or alkyl having 1 to 12 carbon atoms, which is branched or unbranched and which is unsubstituted or substituted one or more times by halogen, hydroxy, cyano, C₁₋₄-alkoxy, oxo or combinations thereof, and wherein optionally one or more —CH₂CH₂— groups is replaced in each case by —CH═CH— or —C≡C—,
cycloalkyl having 3 to 10 carbon atoms, which is unsubstituted or substituted one or more times by halogen, hydroxy, oxo, cyano, alkyl having 1 to 4 carbon atoms, alkoxy having 1 to 4 carbon atoms, or combinations thereof,
cycloalkylalkyl having 4 to 16 carbon atoms, which is unsubstituted or substituted in the cycloalkyl portion and/or the alkyl portion one or more times by halogen, oxo, cyano, hydroxy, C₁₋₄-alkyl, C₁₋₄-alkoxy or combinations thereof,
aryl having 6 to 14 carbon atoms, which is unsubstituted or substituted one or more times by halogen, CF₃, OCF₃, alkyl, hydroxy, alkoxy, nitro, methylenedioxy, ethylenedioxy, cyano, or combinations thereof,
arylalkyl in which the aryl portion has 6 to 14 carbon atoms and the alkyl portion, which is branched or unbranched, has 1 to 5 carbon atoms, wherein the arylalkyl radical is unsubstituted, substituted in the aryl portion one or more times by halogen, CF₃, OCF₃, alkyl, hydroxy, alkoxy, nitro, cyano, methylenedioxy, ethylenedioxy, or combinations thereof, and/or substituted in the alkyl portion one or more times by halogen, oxo, hydroxy, cyano, or combinations thereof, and wherein in the alkyl portion one or more —CH₂CH₂— groups are each optionally replaced by —CH═CH— or —C≡C—, and one or more —CH₂— groups are each optionally replaced by —O— or —NH—,
a partially unsaturated carbocyclic group having 5 to 14 carbon atoms, which is unsubstituted or substituted one or more times by halogen, alkyl, alkoxy, hydroxy, nitro, cyano, oxo, or combinations thereof,
a heterocyclic group, which is saturated, partially saturated or unsaturated, having 5 to 10 ring atoms in which at least 1 ring atom is an N, O or S atom, which is unsubstituted or substituted one or more times by halogen, hydroxy, aryl, alkyl, alkoxy, cyano, trifluoromethyl, nitro, oxo, or combinations thereof, or
a heterocycle-alkyl group, wherein the heterocyclic portion is saturated, partially saturated or unsaturated, and has 5 to 10 ring atoms in which at least 1 ring atom is an N, O or S atom, and the alkyl portion is branched or unbranched and has 1 to 5 carbon atoms, the heterocycle-alkyl group is unsubstituted, substituted one or more times in the heterocyclic portion by halogen, OCF₃, hydroxy, aryl, alkyl, alkoxy, cyano, trifluoromethyl, nitro, oxo, or combinations thereof, and/or substituted in the alkyl portion one or more times by halogen, oxo, hydroxy, cyano, or combinations thereof, and wherein in the alkyl portion one or more —CH₂CH₂— groups are each optionally replaced by —CH═CH— or —C≡C—, and one or more —CH₂— groups are each optionally replaced by —O— or —NH—;
R¹⁸ is H,
alkyl having 1 to 8 carbon atoms, which is unsubstituted or substituted one or more times by halogen, C₁₋₄-alkyl, C₁₋₄-alkoxy, oxo, or combinations thereof, alkylamino or dialkylamino wherein each alkyl portion has independently 1 to 8 carbon atoms,
a partially unsaturated carbocycle-alkyl group wherein the carbocyclic portion has 5 to 14 carbon atoms and the alkyl portion has 1 to 5 carbon atoms, and which is unsubstituted or substituted one or more times by halogen, alkyl, alkoxy, nitro, cyano, oxo, or combinations thereof,
cycloalkyl having 3 to 10 carbon atoms, which is unsubstituted or substituted one or more times by halogen, hydroxy, oxo, cyano, alkoxy, alkyl having 1 to 4 carbon atoms, or combinations thereof,
cycloalkylalkyl having 4 to 16 carbon atoms, which is unsubstituted or substituted in the cycloalkyl portion and/or the alkyl portion one or more times by halogen, oxo, cyano, hydroxy, alkyl, alkoxy or combinations thereof,
aryl having 6 to 14 carbon atoms which is unsubstituted or substituted one or more times by halogen, alkyl, hydroxy, alkoxy, alkoxyalkoxy, nitro, methylenedioxy, ethylenedioxy, trifluoromethyl, amino, aminomethyl, aminoalkyl, aminoalkoxy, dialkylamino, hydroxyalkyl, hydroxamic acid, tetrazole-5-yl, hydroxyalkoxy, carboxy, alkoxycarbonyl, cyano, acyl, alkylthio, alkylsulfinyl, alkylsulfonyl, phenoxy, cycloalkyl, aryl, heteroaryl or combinations thereof,
arylalkyl having 7 to 19 carbon atoms, wherein the aryl portion has 6 to 14 carbon atoms and the alkyl portion, which is branched or unbranched, has 1 to 5 carbon atoms, wherein the arylalkyl radical is unsubstituted or substituted, in the aryl portion, one or more times by halogen, trifluoromethyl, CF₃O, nitro, amino, alkyl, alkoxy, amino, alkylamino, dialkylamino, or combinations thereof, and/or substituted in the alkyl portion by halogen, cyano, alkyl having 1 to 4 carbon atoms, or combinations thereof, wherein in the alkyl portion one or more —CH₂CH₂— groups are each optionally replaced by —CH═CH— or —C≡C—, and/or one or more —CH₂— groups are each optionally replaced by —O— or —NH—,
a heterocyclic group, which is saturated, partially saturated or unsaturated, having 5 to 10 ring atoms in which at least 1 ring atom is an N, O or S atom, which is unsubstituted or substituted one or more times by halogen, alkyl, hydroxy, alkoxy, alkoxyalkoxy, nitro, methylenedioxy, ethylenedioxy, trifluoromethyl, amino, aminomethyl, aminoalkyl, aminoalkoxy, dialkylamino, hydroxyalkyl, hydroxamic acid, tetrazole-5-yl, hydroxyalkoxy, carboxy, alkoxycarbonyl, cyano, acyl, alkylthio, alkylsulfinyl, alkylsulfonyl, phenoxy, cycloalkyl, aryl, heteroaryl or combinations thereof, or
a heterocycle-alkyl group, wherein the heterocyclic portion is saturated, partially saturated or unsaturated, and has 5 to 10 ring atoms in which at least 1 ring atom is an N, O or S atom, and the alkyl portion, which is branched or unbranched, has 1 to 5 carbon atoms, the heterocycle-alkyl group is unsubstituted, substituted one or more times in the heterocyclic portion by halogen, alkyl, alkoxy, cyano, trifluoromethyl, CF₃O, nitro, oxo, amino, alkylamino, dialkylamino, or combinations thereof and/or substituted one or more times in the alkyl portion by halogen, cyano, alkyl having 1 to 4 carbon atoms, or combinations thereof;
M is a single bond or a divalent aliphatic radical having 1 to 8 carbon atoms wherein one or more —CH₂— groups are each optionally replaced by —O—, —S—, —SO—, —SO₂—, —NR¹⁹—, —SO₂NR¹⁹—, —NR¹⁹SO₂—, —CO—, —CO₂—, —NR¹⁹CO—, —CONR¹⁹—, —NHCONH—, —OCONH, —NHCOO—, —SCONH—, —SCSNH—, —NHCSNH—, —CONHSO₂— or —SO₂NHCO—; and
R¹⁹ is H,
alkyl having 1 to 8 carbon atoms, which is branched or unbranched and which is unsubstituted or substituted one or more times with halogen, C₁₋₄-alkyl, C₁₋₄-alkoxy, oxo, or combinations thereof,
arylalkyl having 7 to 19 carbon atoms, wherein the aryl portion has 6 to 14 carbon atoms and the alkyl portion, which is branched or unbranched, has 1 to 5 carbon atoms, wherein the arylalkyl radical is unsubstituted or substituted, in the aryl portion, one or more times by halogen, trifluoromethyl, CF₃O, nitro, amino, alkyl, alkoxy, amino, alkylamino, dialkylamino, or combinations thereof, and/or substituted in the alkyl portion by halogen, cyano, alkyl having 1 to 4 carbon atoms, or combinations thereof, wherein in the alkyl portion one or more —CH₂CH₂— groups are each optionally replaced by —CH═CH— or —C≡C—, and/or one or more —CH₂— groups are each optionally replaced by —O— or —NH—, or
aryl having 6 to 14 carbon atoms and which is unsubstituted or substituted one or more times by halogen, alkyl, hydroxy, alkoxy, alkoxyalkoxy, nitro, methylenedioxy, ethylenedioxy, trifluoromethyl, amino, aminomethyl, aminoalkyl, aminoalkoxy dialkylamino, hydroxyalkyl, hydroxamic acid, tetrazole-5-yl, hydroxyalkoxy, carboxy, alkoxycarbonyl, cyano, acyl, alkylthio, alkylsulfinyl, alkylsulfonyl, or combinations thereof; and
R²⁰ is H or alkyl having 1 to 4 carbon atoms, which is branched or unbranched and which is unsubstituted or substituted one or more times by halogen; or
a pharmaceutically acceptable salt or solvate thereof, or solvate of a pharmaceutically acceptable salt thereof;
wherein if the compound exhibits chirality it can be in the form of a mixture of enantiomers such as a racemate or a mixture of diastereomers, or can be in the form of a single enantiomer or a single diastereomer;
with the provisos that:
when R¹⁴ is pyridinylmethyl, R¹³ is other than unsubstituted or substituted piperidinyl,
and said compound is not:
6-[(3,4-dimethoxyphenyl)(2-furanylmethyl)amino]-1,2,4-triazine-3,5(2H,4H)dione,
N-(6-(cyclopentyloxy)-5-methoxy-2-pyridinyl]-N-4-piperidinyl-5-pyrimidinemethanamine,
N,N-dibutyl-4-[(3-chloro-4-methoxyphenyl)(1-propyl-4-piperidinyl)amino]benzamide,
4-[(3-chloro-4-methoxyphenyl)(1-propyl-4-piperidinyl)amino]-N,N-bis(1-methylethyl)benzamide,
4-[(3-chloro-4-methoxyphenyl)(1-propyl-4-piperidinyl)amino]-N,N-dipropylbenzamide,
4-[(3-chloro-4-methoxyphenyl)(1-propyl-4-piperidinyl)amino]-N,N-diethylbenzamide,
N-(3,4-dimethylphenyl)-N-4-morpholinyl-4-morpholinamine],
N-(3,4-dimethylphenyl)-N-1-piperidinyl-1-piperidinamine, or
6-(cyclopentyloxy)-5-methoxy-N-phenyl-N-piperidin-4-ylpyridin-2-amine,
or a pharmaceutically acceptable salt thereof, or solvate thereof, or solvate of a pharmaceutically acceptable salts thereof.

15. A compound according to claim 14, wherein R¹⁷ is H, alkyl which is unsubstituted or substituted, cycloalkylalkyl which is unsubstituted or substituted, aryl which is unsubstituted or substituted, arylalkyl which is unsubstituted or substituted, a partially unsaturated carbocyclic group which is unsubstituted or substituted, a heterocyclic group which is unsubstituted or substituted, or a heterocycle-alkyl group which is unsubstituted or substituted; and R¹⁴ is cycloalkyl which is unsubstituted or substituted, aryl which is unsubstituted or substituted one or more times by halogen, alkyl, alkenyl, alkynyl, hydroxy, alkoxy, alkoxyalkoxy, nitro, methylenedioxy, ethylenedioxy, trifluoromethyl, OCF₃, amino, aminoalkyl, aminoalkoxy, dialkylamino, hydroxyalkyl, hydroxamic acid, hydroxyalkoxy, carboxy, carboxyalkyl, alkoxycarbonyl, cyano, acyl, alkylthio, alkylsulfinyl, alkylsulfonyl, phenoxy, trialkylsilyloxy, or combinations thereof, heteroaryl which is unsubstituted or substituted one or more times by halogen, alkyl, alkenyl, alkynyl, hydroxy, alkoxy, alkoxyalkoxy, nitro, methylenedioxy, ethylenedioxy, trifluoromethyl, OCF₃, amino, aminoalkyl, aminoalkoxy, dialkylamino, hydroxyalkyl, hydroxamic acid, hydroxyalkoxy, carboxy, carboxyalkyl, alkoxycarbonyl, cyano, acyl, alkylthio, alkylsulfinyl, alkylsulfonyl, phenoxy, trialkylsilyloxy, or combinations thereof, or a heterocyclic group other than morpholinyl which is substituted or unsubstituted.

16. A compound according to claim 14, wherein R¹⁴ is cycloalkyl, aryl, heteroaryl, or a heterocyclic group.

17. A compound according to claim 14, wherein R¹¹ is OR¹⁶ and/or R¹² in Formula II is OR¹⁷.

18. (canceled)

19. (canceled)

20. A compound according to claim 14, wherein R¹¹ is halogen or OR¹⁶, and R¹⁶ is alkyl or halogenated alkyl.

21. A compound according to claim 14, wherein E is N or CH; R¹¹ is OR⁶; R¹² is OR¹⁷; R¹³ is a fully saturated heterocyclic group having 5 to 10 ring atoms, particularly 5-8 ring atoms, which is substituted or unsubstituted, in which at least 1 ring atom is N, such as substituted or unsubstituted piperidinyl, or substituted or unsubstituted pyrrolidinyl; R¹⁴ is aryl or heteroaryl, each of which is substituted or unsubstituted; R¹⁶ is alkyl, or halogenated alkyl; R¹⁷ is alkyl, cycloalkyl, or cycloalkylalkyl, and R¹⁴ is other than unsubstituted phenyl when R¹³ is substituted or unsubstituted piperidinyl.

22. (canceled)

23. (canceled)

24. (canceled)

25. (canceled)

26. (canceled)

27. (canceled)

28. (canceled)

29. A compound according to Formula III: 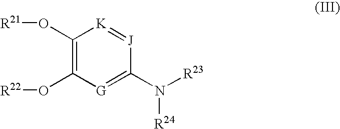
wherein
G, J, and K are each, independently, N or CR²⁵;
R²¹ is alkyl having 1 to 4 carbon atoms, which is branched or unbranched and which is unsubstituted or substituted one or more times by halogen;
R²² is alkyl having 1 to 12 carbon atoms, which is branched or unbranched and which is unsubstituted or substituted one or more times by halogen, hydroxy, cyano, C₁₋₄-alkoxy, oxo or combinations thereof, and wherein optionally one or more —CH₂CH₂— groups is replaced in each case by —CH═CH— or —C≡C—,
cycloalkyl having 3 to 10 carbon atoms, which is unsubstituted or substituted one or more times by halogen, hydroxy, oxo, cyano, alkyl having 1 to 4 carbon atoms, alkoxy having 1 to 4 carbon atoms, or combinations thereof,
cycloalkylalkyl having 4 to 16 carbon atoms, which is unsubstituted or substituted in the cycloalkyl portion and/or the alkyl portion one or more times by halogen, oxo, cyano, hydroxy, C₁₋₄-alkyl, C₁₋₄-alkoxy or combinations thereof,
aryl having 6 to 14 carbon atoms, which is unsubstituted or substituted one or more times by halogen, CF₃, OCF₃, alkyl, hydroxy, alkoxy, nitro, methylenedioxy, ethylenedioxy, cyano, or combinations thereof,
arylalkyl in which the aryl portion has 6 to 14 carbon atoms and the alkyl portion, which is branched or unbranched, has 1 to 5 carbon atoms, wherein the arylalkyl radical is unsubstituted or is substituted in the aryl portion one or more times by halogen, CF₃, OCF₃, alkyl, hydroxy, alkoxy, nitro, cyano, methylenedioxy, ethylenedioxy, or combinations thereof, and wherein in the alkyl portion one or more —CH₂CH₂— groups are each optionally replaced by —CH═CH— or —C≡C—, and one or more —CH₂— groups are each optionally replaced by —O— or —NH— and/or the alkyl portion is optionally substituted by halogen, oxo, hydroxy, cyano, or combinations thereof,
a partially unsaturated carbocyclic group having 5 to 14 carbon atoms, which is unsubstituted or substituted one or more times by halogen, alkyl, alkoxy, hydroxy, nitro, cyano, oxo, or combinations thereof,
a heterocyclic group, which is saturated, partially saturated or unsaturated, having 5 to 10 ring atoms in which at least 1 ring atom is a N, O or S atom, which is unsubstituted or substituted one or more times by halogen, hydroxy, aryl, alkyl, alkoxy, cyano, trifluoromethyl, nitro, oxo, or combinations thereof, or
a heterocycle-alkyl group, wherein the heterocyclic portion is saturated, partially saturated or unsaturated, and has 5 to 10 ring atoms in which at least 1 ring atom is a N, O or S atom, and the alkyl portion is branched or unbranched and has 1 to 5 carbon atoms, the heterocycle-alkyl group is unsubstituted or substituted one or more times in the heterocyclic portion by halogen, OCF₃, hydroxy, aryl, alkyl, alkoxy, cyano, trifluoromethyl, nitro, oxo, or combinations thereof, wherein in the alkyl portion one or more —CH₂CH₂— groups are each optionally replaced by —CH═CH— or —C≡C—, and one or more —CH₂— groups are each optionally replaced by —O— or —NH— and/or the alkyl portion is optionally substituted by halogen, oxo, hydroxy, cyano, or combinations thereof;
R²³ a non-aromatic heterocyclic group, which is fully saturated or partially saturated, having 5 to 10 ring atoms in which at least 1 ring atom is an N, O or S atom, which is unsubstituted or substituted one or more times by halogen, alkyl, hydroxy, alkoxy, alkoxyalkoxy, hydroxyalkyl-alkoxy, dihydroxyalkyl-alkoxy, nitro, oxo, methylenedioxy, ethylenedioxy, trifluoromethyl, OCF₃, amino, aminomethyl, aminoalkyl, aminoalkoxy, dialkylamino, hydroxyalkyl, dihydroxyalkyl, hydroxamic acid, tetrazole-5-yl, hydroxyalkoxy, carboxy, alkoxycarbonyl, cyano, acyl, alkylthio, alkylsulfinyl, alkylsulfonyl, phenylsulfonyl, phenoxy, cycloalkyl, aryl, heteroaryl or combinations thereof,
R²⁴ is cycloalkyl having 3 to 10 carbon atoms, which is unsubstituted or substituted one or more times by halogen, hydroxy, oxo, cyano, alkyl having 1 to 4 carbon atoms, alkoxy having 1 to 4 carbon atoms, or combinations thereof,
aryl having 6 to 14 carbon atoms and which is unsubstituted or substituted one or more times by halogen, alkyl, alkenyl, alkynyl, hydroxy, alkoxy, alkoxyalkoxy, nitro, methylenedioxy, ethylenedioxy, trifluoromethyl, OCF₃, amino, aminoalkyl, aminoalkoxy, dialkylamino, amido, hydroxyalkyl, hydroxamic acid, pyrrolyl, tetrazole-5-yl, 2(-heterocycle)tetrazole-5-yl, hydroxyalkoxy, carboxy, carboxyalkyl, alkoxycarbonyl, cyano, acyl, alkylthio, alkylsulfinyl, alkylsulfonyl, phenoxy, trialkylsilyloxy, R²⁶-Q-, or combinations thereof,
heteroaryl having 5 to 10 ring atoms in which at least 1 ring atom is a heteroatom, which is unsubstituted or substituted one or more times by halogen, alkyl, hydroxy, alkoxy, alkoxyalkoxy, nitro, methylenedioxy, ethylenedioxy, trifluoromethyl, amino, aminomethyl, aminoalkyl, aminoalkoxy, dialkylamino, hydroxyalkyl, hydroxamic acid, tetrazole-5-yl, hydroxyalkoxy, carboxy, carboxyalkyl, alkoxycarbonyl, cyano, acyl, alkylthio, alkylsulfinyl, alkylsulfonyl, phenoxy, trialkylsilyloxy, R²⁶-Q-, or combinations thereof,
a heterocyclic group, which is saturated or partially saturated, having 5 to 10 ring atoms in which at least 1 ring atom is an N, O or S atom, which is unsubstituted or substituted one or more times by halogen, alkyl, hydroxy, alkoxy, alkoxyalkoxy, nitro, oxo, methylenedioxy, ethylenedioxy, trifluoromethyl, OCF₃, amino, aminomethyl, aminoalkyl, aminoalkoxy, dialkylamino, hydroxyalkyl, hydroxamic acid, tetrazole-5-yl, hydroxyalkoxy, carboxy, alkoxycarbonyl, cyano, acyl, alkylthio, alkylsulfinyl, alkylsulfonyl, phenylsulfonyl, phenoxy, cycloalkyl, aryl, heteroaryl or combinations thereof, and
a heterocycle-alkyl group, wherein the heterocyclic portion is saturated, partially saturated or unsaturated, and has 5 to 10 ring atoms in which at least 1 ring atom is an N, O or S atom, and the alkyl portion is branched or unbranched and has 1 to 5 carbon atoms, the heterocycle-alkyl group is unsubstituted, substituted one or more times in the heterocyclic portion by halogen, alkyl, hydroxy, alkoxy, alkoxyalkoxy, nitro, oxo, methylenedioxy, ethylenedioxy, trifluoromethyl, OCF₃, amino, aminomethyl, aminoalkyl, aminoalkoxy, dialkylamino, hydroxyalkyl, hydroxamic acid, tetrazole-5-yl, hydroxyalkoxy, carboxy, alkoxycarbonyl, cyano, acyl alkylthio, alkylsulfinyl, alkylsulfonyl, phenylsulfonyl, phenoxy, cycloalkyl, aryl, heteroaryl or combinations thereof, and/or substituted in the alkyl portion one or more times by halogen, oxo, hydroxy, cyano, or combinations thereof, and wherein in the alkyl portion one or more —CH₂CH₂— groups are each optionally replaced by —CH═CH— or —C≡C—, and one or more —CH₂— groups are each optionally replaced by —O— or —NH—;
R²⁵ is H, halogen, alkyl having 1 to 4 carbon atoms, halogenated alkyl having 1 to 4 carbon atoms, alkoxy having 1 to 4 carbon atoms, or halogenated alkoxy having 1 to 4 carbon atoms;
R²⁶ is H,
alkyl having 1 to 8 carbon atoms, which is unsubstituted or substituted one or more times by halogen, C₁₋₄-alkyl, C₁₋₄-alkoxy, oxo, or combinations thereof,
alkylamino or dialkylamino wherein each alkyl portion has independently 1 to 8 carbon atoms,
a partially unsaturated carbocycle-alkyl group wherein the carbocyclic portion has 5 to 14 carbon atoms and the alkyl portion has 1 to 5 carbon atoms, and which is unsubstituted or substituted one or more times by halogen, alkyl, alkoxy, nitro, cyano, oxo, or combinations thereof,
cycloalkyl having 3 to 10 carbon atoms, which is unsubstituted or substituted one or more times by halogen, hydroxy, oxo, cyano, alkoxy, alkyl having 1 to 4 carbon atoms, or combinations thereof,
cycloalkylalkyl having 4 to 16 carbon atoms, which is unsubstituted or substituted in the cycloalkyl portion and/or the alkyl portion one or more times by halogen, oxo, cyano, hydroxy, alkyl, alkoxy or combinations thereof,
aryl having 6 to 14 carbon atoms which is unsubstituted or substituted one or more times by halogen, alkyl, hydroxy, alkoxy, alkoxyalkoxy, nitro, methylenedioxy, ethylenedioxy, trifluoromethyl, amino, aminomethyl, aminoalkyl, aminoalkoxy, dialkylamino, hydroxyalkyl, hydroxamic acid, tetrazole-5-yl, hydroxyalkoxy, carboxy, alkoxycarbonyl, cyano, acyl, alkylthio, alkylsulfinyl, alkylsulfonyl, phenoxy, cycloalkyl, aryl, heteroaryl or combinations thereof,
arylalkyl having 7 to 19 carbon atoms, wherein the aryl portion has 6 to 14 carbon atoms and the alkyl portion, which is branched or unbranched, has 1 to 5 carbon atoms, wherein the arylalkyl radical is unsubstituted or substituted, in the aryl portion, one or more times by halogen, trifluoromethyl, CF₃O, nitro, amino, alkyl, alkoxy, amino, alkylamino, dialkylamino, or combinations thereof, and/or substituted in the alkyl portion by halogen, cyano, alkyl having 1 to 4 carbon atoms, or combinations thereof, wherein in the alkyl portion one or more —CH₂CH₂— groups are each optionally replaced by —CH═CH— or —C≡C—, and/or one or more —CH₂— groups are each optionally replaced by —O— or —NH—,
a heterocyclic group, which is saturated, partially saturated or unsaturated, having 5 to 10 ring atoms in which at least 1 ring atom is an N, O or S atom, which is unsubstituted or substituted one or more times by halogen, alkyl, hydroxy, alkoxy, alkoxyalkoxy, nitro, methylenedioxy, ethylenedioxy, trifluoromethyl, amino, aminomethyl, aminoalkyl, aminoalkoxy, dialkylamino, hydroxyalkyl, hydroxamic acid, tetrazole-5-yl, hydroxyalkoxy, carboxy, alkoxycarbonyl, or
a heterocycle-alkyl group, wherein the heterocyclic portion is saturated, partially saturated or unsaturated, and has 5 to 10 ring atoms in which at least 1 ring atom is an N, O or S atom, and the alkyl portion, which is branched or unbranched, has 1 to 5 carbon atoms, the heterocycle-alkyl group is unsubstituted, substituted one or more times in the heterocyclic portion by halogen, alkyl, alkoxy, cyano, trifluoromethyl, CF₃O, nitro, oxo, amino, alkylamino, dialkylamino, or combinations thereof and/or substituted one or more times in the alkyl portion by halogen, cyano, alkyl having 1 to 4 carbon atoms, or combinations thereof;
Q is a single bond or a divalent aliphatic radical having 1 to 8 carbon atoms wherein one or more —CH₂— groups are each optionally replaced by —O—, —S—, —SO—, —SO₂—, —NR²⁷—, —SO₂NR²⁷—, —NR²⁷SO₂—, —CO—, —CO₂—, —NR²⁷CO—, —CONR²⁷—, —NHCONH—, —OCONH, —NHCOO—, —SCONH—, —SCSNH—, —NHCSNH—, —CONHSO₂— or —SO₂NHCO—; and
R²⁷ is H,
alkyl having 1 to 8 carbon atoms, which is branched or unbranched and which is unsubstituted or substituted one or more times with halogen, C₁₋₄-alkyl, C₁₋₄-alkoxy, oxo, or combinations thereof,
arylalkyl having 7 to 19 carbon atoms, wherein the aryl portion has 6 to 14 carbon atoms and the alkyl portion, which is branched or unbranched, has 1 to 5 carbon atoms, wherein the arylalkyl radical is unsubstituted or substituted, in the aryl portion, one or more times by halogen, trifluoromethyl, CF₃O, nitro, amino, alkyl, alkoxy, amino, alkylamino, dialkylamino, or combinations thereof, and/or substituted in the alkyl portion by halogen, cyano, alkyl having 1 to 4 carbon atoms, or combinations thereof, wherein in the alkyl portion one or more —CH₂CH₂— groups are each optionally replaced by —CH═CH— or —C≡C—, and/or one or more —CH₂— groups are each optionally replaced by —O— or —NH—, or
aryl having 6 to 14 carbon atoms and which is unsubstituted or substituted one or more times by halogen, alkyl, hydroxy, alkoxy, alkoxyalkoxy, nitro, methylenedioxy, ethylenedioxy, trifluoromethyl, amino, aminomethyl, aminoalkyl, aminoalkoxy dialkylamino, hydroxyalkyl, hydroxamic acid, tetrazole-5-yl, hydroxyalkoxy, carboxy, alkoxycarbonyl, cyano, acyl, alkylthio, alkylsulfinyl, alkylsulfonyl, or combinations thereof; or
a pharmaceutically acceptable salt or solvate thereof, or a solvate of a pharmaceutically acceptable salt thereof;
wherein if the compound exhibits chirality it can be in the form of a mixture of enantiomers such as a racemate or a mixture of diastereomers, or can be in the form of a single enantiomer or a single diastereomer;
with the provisos that:
when R¹⁴ is pyridinylmethyl, R¹³ is other than unsubstituted or substituted piperidinyl, and
said compound is not:
6-[(3,4-dimethoxyphenyl)(2-furanylmethyl)amino]-1,2,4-triazine-3,5(2H,4H)dione,
N-(6-(cyclopentyloxy)-5-methoxy-2-pyridinyl]-N-4-piperidinyl-5-pyrimidinemethanamine,
6-(cyclopentyloxy)-5-methoxy-N-phenyl-N-piperidin-4-ylpyridin-2-amine, or
a pharmaceutically acceptable salt thereof, or solvate thereof, or solvate of a pharmaceutically acceptable salt thereof.

30. A compound according to claim 29, wherein R²² is alkyl which is unsubstituted or substituted, cycloalkylalkyl which is unsubstituted or substituted, aryl which is unsubstituted or substituted, arylalkyl which is unsubstituted or substituted, a partially unsaturated carbocyclic group which is unsubstituted or substituted, a heterocyclic group which is unsubstituted or substituted, or a heterocycle-alkyl group which is unsubstituted or substituted; and R²⁴ is cycloalkyl which is unsubstituted or substituted, aryl which is unsubstituted or substituted one or more times by halogen, alkyl, alkenyl, alkynyl, hydroxy, alkoxy, alkoxyalkoxy, nitro, methylenedioxy, ethylenedioxy, trifluoromethyl, OCF₃, amino, aminoalkyl, aminoalkoxy, dialkylamino, hydroxyalkyl, hydroxamic acid, hydroxyalkoxy, carboxy, carboxyalkyl, alkoxycarbonyl, cyano, acyl, alkylthio, alkylsulfinyl, alkylsulfonyl, phenoxy, trialkylsilyloxy, or combinations thereof, heteroaryl which is unsubstituted or substituted one or more times by halogen, alkyl, alkenyl, alkynyl, hydroxy, alkoxy, alkoxyalkoxy, nitro, methylenedioxy, ethylenedioxy, trifluoromethyl, OCF₃, amino, aminoalkyl, aminoalkoxy, dialkylamino, hydroxyalkyl, hydroxamic acid, hydroxyalkoxy, carboxy, carboxyalkyl, alkoxycarbonyl, cyano, acyl, alkylthio, alkylsulfinyl, alkylsulfonyl, phenoxy, trialkylsilyloxy, or combinations thereof, or a heterocyclic group other than morpholinyl which is substituted or unsubstituted.

31. A compound according to claim 29, wherein R²⁴ is cycloalkyl, aryl, heteroaryl, or a heterocyclic group.

32. A compound according to claim 29, wherein one of G, J, and K is N and the others are CR²⁵.

33. A compound according to claim 29, wherein each of G, J, and K is CR²⁵.

34. A compound according to claim 29, wherein R²¹ is alkyl, or halogenated alkyl.

35. A compound according to claim 29, wherein R²² is alkyl or cycloalkylalkyl.

36. A compound according to claim 29, wherein R²³ is substituted or unsubstituted piperidinyl, or substituted or unsubstituted pyrrolidinyl.

37. A compound according to claim 29, wherein R²⁴ is unsubstituted phenyl or phenyl substituted by halogen, alkyl, carboxy, alkoxy, dialkylamino, CONH₂ and/or cyano.

38. A compound according to claim 31, wherein R²⁶ is methyl, ethyl, propyl or phenyl, which in each case is unsubstituted or substituted.

39. A compound according to claim 29, wherein R²⁷ is H, alkyl having 1 to 4 carbon atoms, or aryl.

40. A compound according to claim 29, wherein R²⁵ is H, F or methyl.

41. A compound according to claim 29, wherein R²⁴ is phenyl substituted by halogen, alkyl, carboxy, alkoxy, alkylamino, dialkylamino, CONH₂, nitro and/or cyano when R²³ is substituted or unsubstituted piperidinyl, when G is CH, then K is other than CR²⁵ in which R²⁵ is alkoxy having 1 to 4 carbon atoms, and when K is CH, then G is other than CR²⁵ in which R²⁵ is alkoxy having 1 to 4 carbon atoms.

42. (canceled)

43. A compound selected from:
N-(3-Chlorophenyl)-6-isopropoxy-5-methoxy-N-piperidin-4-ylpyridin-2-amine,
N-(3-chlorophenyl)-5-(difluoromethoxy)-6-ethoxy-N-piperidin-4-ylpyridin-2-amine,
5-(difluoromethoxy)-6-methoxy-N-(4-methoxyphenyl)-N-piperidin-4-ylpyridin-2-amine,
4-[(6-ethoxy-5-methoxypyridin-2-yl)(piperidin-4-yl)amino]benzonitrile,
4-[(6-ethoxy-5-methoxypyridin-2-yl)(piperidin-4-yl)amino]benzamide,
4-[[6-(cyclohexyloxy)-5-methoxypyridin-2-yl](piperidin-4-yl)amino]benzoic acid,
6-(cyclopropylmethoxy)-5-methoxy-N-phenyl-N-piperidin-3-ylpyridin-2-amine,
6-ethoxy-5-methoxy-N-piperidin-4-yl-N-3-thienylpyridin-2-amine,
6-ethoxy-5-methoxy-N-(4-methylphenyl)-N-pyrrolidin-3-ylpyridin-2-amine,
N-(6-isobutoxy-5-methoxypyridin-2-yl)-N′,N′-dimethyl-N-piperidin-4-ylbenzene-1,4-diamine, and
N-(3-chlorophenyl)-N-[4-(difluoromethoxy)-3-methoxyphenyl]piperidin-4-amine,
and pharmaceutically acceptable salts thereof, solvates thereof, and solvates of pharmaceutically acceptable salts thereof;
wherein if the compound exhibits chirality it can be in the form of a mixture of enantiomers such as a racemate or a mixture of diastereomers, or can be in the form of a single enantiomer or a single diastereomer.

44. A compound according to claim 43, wherein said compound is in the form of a hydrochloride, an oxalate, a hydroformate or a trifluoroacetate salt.

45. A pharmaceutical composition comprising a compound according to claim 1, and a pharmaceutically acceptable carrier.

46. A composition according to claim 45, wherein said composition contains 0.1-50 mg of said compound.

47. A composition according to claim 45, wherein said composition further comprises one or more additional pharmaceutical agent or agents selected from calcium channel blockers, cholinergic drugs, adenosine receptor modulators, ampakines, NMDA-R modulators, mGluR modulators, cholinesterase inhibitors, selective serotonin reuptake inhibitors, and combinations thereof.

48. (canceled)

49. A method for enhancing cognition in a patient in whom such enhancement is desired comprising administering to said patient an effective amount of a compound according to claim 1.

50. (canceled)

51. (canceled)

52. A method of treating a patient suffering from cognition impairment or decline comprising administering to said patient an effective amount of a compound according to claim 1.

53. (canceled)

54. A method according to claim 52, wherein said patient is human and is suffering from memory impairment.

55. (canceled)

56. (canceled)

57. (canceled)

58. (canceled)

59. (canceled)

60. (canceled)

61. A method of treating a patient suffering from an allergic or inflammatory disease comprising administering to said patient an effective amount of a compound according to claim 1.

62. A method for treating a patient suffering from schizophrenia, bipolar or manic depression, major depression, drug addiction and/or morphine dependence, comprising administering to said patient an effective amount of a compound according to claim 1.

63. (canceled)

64. (canceled)

65. (canceled)

66. (canceled)

67. (canceled)

68. (canceled)

69. A method for treating a patient suffering from psychosis characterized by elevated levels of PDE 4, wherein said psychosis is a form of depression, comprising administering to said patient an effective amount of a compound according to claim 1.

70. (canceled)

71. (canceled)

72. (canceled)

73. (canceled)
